(19) United States
(12) Patent Application Publication
(10) Pub. No.: US 2020/0262865 A1
(43) Pub. Date: Aug. 20, 2020

(54) COMPOSITIONS AND METHODS FOR TREATING CLOSTRIDIUM ASSOCIATED DISEASES

(71) Applicants: Alexander KHORUTS, Minneapolis, MN (US); Michael J. SADOWSKY, Roseville, MN (US); Peter DOSA, Vadnais Heights, MN (US); Kristen STOLTZ, Minneapolis, MN (US); REGENTS OF THE UNIVERSITY OF MINNESOTA, Minneapolis, MN (US)

(72) Inventors: Alexander Khoruts, Minneapolis, MN (US); Michael J. Sadowsky, Roseville, MN (US); Peter Dosa, Vadnais Heights, MN (US); Kristen Layne Stoltz, Minneapolis, MN (US)

(21) Appl. No.: 15/998,547

(22) PCT Filed: Feb. 15, 2017
(86) PCT No.: PCT/US2017/017888

§ 371 Date: Aug. 15, 2018

Related U.S. Application Data
(60) Provisional application No. 62/295,367, filed on Feb. 15, 2016.

(51) Int. Cl. C07J 43/00; C07J 9/00; A61P 31/04

(52) U.S. Cl. CPC C07J 43/003; A61P 31/04; C07J 9/005

(57) ABSTRACT

The present disclosure provides compounds for preventing, treating, and/or reducing the risk of developing a Clostridium-associated disease in a mammalian subject. Also provided are pharmaceutically acceptable salts of such compounds and compositions that include such compounds and/or pharmaceutically acceptable salts thereof.

This application claims the benefit of U.S. Provisional Application No. 62/295,367, filed Feb. 15, 2016, which is herein incorporated by reference in its entirety.

### BACKGROUND

Clostridium difficile is an anaerobic, spore-forming, gram-positive bacterium that causes a potentially fatal infection of the colon. C. difficile infection (CDI) is among the most common hospital-acquired infections, and in recent years CDI has also become increasingly acquired in the community. The infection affects approximately 500,000 patients annually and leads to the death of 30,000 patients per year in the US alone. CDI typically occurs after the disruption of gut microbiota following one or more courses of antibiotics and subsequent ingestion of C. difficile spores from the surrounding environment. The current standard of care also relies on antibiotics. However, this strategy can perpetuate recurrence of CDI because it fails to eliminate C. difficile spores and results in further suppression of indigenous microbiota. After the initial infection and antibiotic treatment, relapse can occur in 20 to 40% of patients, with a higher risk of recurrence associated with each episode of infection. A large fraction of patients ultimately develop an indefinite syndrome of recurrent CDI (R-CDI), which becomes refractory to eradication with antibiotics alone.

There is a continuing need for new compositions and methods for preventing, treating, and/or reducing the risk of developing Clostridium difficile-associated diseases.

### SUMMARY

In one aspect, the present disclosure provides compounds and compositions for preventing, treating, and/or reducing the risk of developing a Clostridium-associated disease (e.g., a Clostridium difficile-associated disease) in a mammalian subject.

In one embodiment, a compound for preventing, treating, and/or reducing the risk of developing a Clostridium-associated disease in a mammalian subject is of the formula:

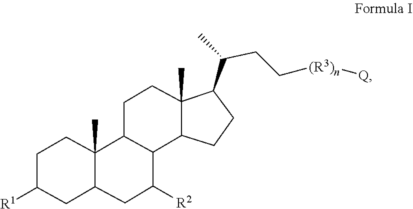

wherein: R¹ and R² are each independently selected from the group consisting of —H, —OR⁶, and —OC(O)R⁸; each R⁶ is independently selected from the group consisting of —H, a C1-C10 straight chain or branched chain alkyl or cycloalkyl group, —SO₃H, and a pharmaceutically acceptable salt of —SO₃H; R⁸ represents —H or a C1-C10 straight chain or branched chain alkyl or cycloalkyl group; R³ represents —C(O)— or a C1-C10 straight chain or branched chain alkylene or cycloalkylene group; n=0 or 1; and Q represents a heterocyclic ring, with the proviso that if R² is H, then Q does not represent a tetrazole.

In certain embodiments, the present disclosure further provides pharmaceutically acceptable salts of a compound of Formula I. In certain embodiments, the present disclosure further provides compositions that include a compound of Formula I and/or a pharmaceutically acceptable salt thereof.

In another embodiment, a compound for preventing, treating, and/or reducing the risk of developing a Clostridium-associated disease in a mammalian subject is of the formula:

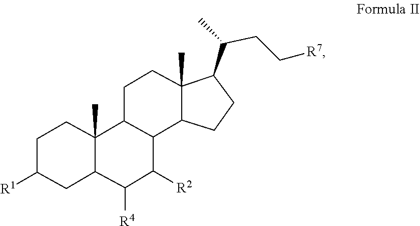

wherein: R¹ and R² are each independently selected from the group consisting of —H, —OR⁶, and —OC(O)R⁸; R⁴ represents —OR⁸ or —OC(O)R⁸; each R⁶ is independently selected from the group consisting of —H, a C1-C10 straight chain or branched chain alkyl or cycloalkyl group, —SO₃H, and a pharmaceutically acceptable salt of —SO₃H; R⁷ represents —C(O)OR⁸ or —(R³)ₙ-Q; each R⁸ independently represents —H or a C1-C10 straight chain or branched chain alkyl or cycloalkyl group; R³ represents —C(O)— or a C1-C10 straight chain or branched chain alkylene or cycloalkylene group; n=0 or 1; and Q represents a heterocyclic ring. In certain embodiments, the present disclosure further provides pharmaceutically acceptable salts of a compound of Formula II. In certain embodiments, the present disclosure further provides compositions that include a compound of Formula II and/or a pharmaceutically acceptable salt thereof.

In another embodiment, a compound for preventing, treating, and/or reducing the risk of developing a Clostridium-associated disease in a mammalian subject is of the formula:

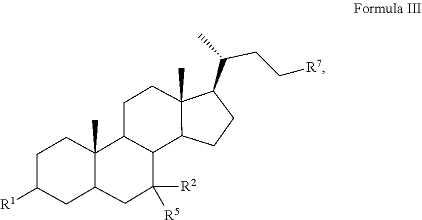

wherein: R¹ represents H, —OR⁶, or —OC(O)R⁸; R² represents —OR⁶ or —OC(O)R⁶; R⁵ represents F or a C1-C10 straight chain or branched chain alkyl or cycloalkyl group; each R⁶ is independently selected from the group consisting of —H, a C1-C10 straight chain or branched chain alkyl or cycloalkyl group, —SO₃H, and a pharmaceutically acceptable salt of —SO₃H; R⁷ represents —C(O)OR⁸ or —(R³)ₙ-Q; each R⁸ independently represents —H or a C1-C10 straight chain or branched chain alkyl or cycloalkyl group; R³ represents —C(O)— or a C1-C10 straight chain or branched chain alkylene or cycloalkylene group; n=0 or 1; and Q represents a heterocyclic ring. In certain embodiments, the present disclosure further provides pharmaceutically acceptable salts of a compound of Formula III. In certain embodiments, the present disclosure further provides compositions that include a compound of Formula III and/or a pharmaceutically acceptable salt thereof.

In another embodiment, a compound for preventing, treating, and/or reducing the risk of developing a Clostridium-associated disease in a mammalian subject is of the formula:

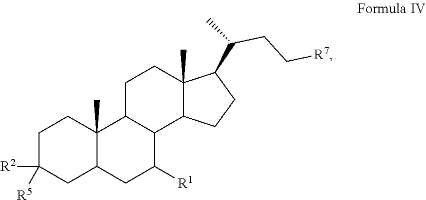

wherein: R¹ represents H, —OR⁶, or —OC(O)R⁸; R² represents —OR⁶ or —OC(O)R⁶; R⁵ represents F or a C1-C10 straight chain or branched chain alkyl or cycloalkyl group; each R⁶ is independently selected from the group consisting of —H, a C1-C10 straight chain or branched chain alkyl or cycloalkyl group, —SO₃H, and a pharmaceutically acceptable salt of —SO₃H; R⁷ represents —C(O)OR⁸ or —(R³)ₙ-Q; each R⁸ independently represents —H or a C1-C10 straight chain or branched chain alkyl or cycloalkyl group; R³ represents —C(O)— or a C1-C10 straight chain or branched chain alkylene or cycloalkylene group; n=0 or 1; and Q represents a heterocyclic ring. In certain embodiments, the present disclosure further provides pharmaceutically acceptable salts of a compound of Formula IV. In certain embodiments, the present disclosure further provides compositions that include a compound of Formula IV and/or a pharmaceutically acceptable salt thereof.

In another embodiment, a compound for preventing, treating, and/or reducing the risk of developing a Clostridium-associated disease in a mammalian subject is of the formula:

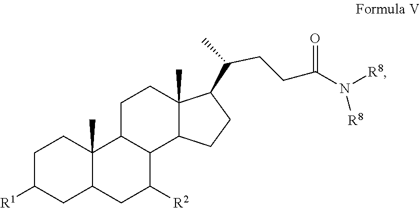

wherein: R¹ and R² are each independently selected from the group consisting of —H, —OR⁶, and —OC(O)R⁸; each R⁶ is independently selected from the group consisting of —H, a C1-C10 straight chain or branched chain alkyl or cycloalkyl group, —SO₃H, and a pharmaceutically acceptable salt of —SO₃H; and each R⁸ independently represents —H or a C1-C10 straight chain or branched chain alkyl or cycloalkyl group; with the proviso that one or both of R¹ and R² represent —OSO₃H or a pharmaceutically acceptable salt of —OSO₃H. In certain embodiments, the present disclosure further provides pharmaceutically acceptable salts of a compound of Formula V. In certain embodiments, the present disclosure further provides compositions that include a compound of Formula V and/or a pharmaceutically acceptable salt thereof.

In another aspect, the present disclosure provides a method of preventing a Clostridium-associated disease in a mammalian subject, including administering to a mammalian subject at risk of developing a Clostridium-associated disease an effective amount of a compound, a pharmaceutically acceptable salt of a compound, or a composition as disclosed herein, prior to or concurrently with an optional administration of an antibiotic.

In another aspect, the present disclosure provides a method of treating a Clostridium-associated disease in a mammalian subject, including administering to a mammalian subject having a Clostridium-associated disease an effective amount of a compound, a pharmaceutically acceptable salt of a compound, or a composition as disclosed herein, prior to or concurrently with an optional administration of an antibiotic.

In another aspect, the present disclosure provides a method of reducing the risk of developing a Clostridium-associated disease in a mammalian subject receiving antibiotic therapy, including administering to a mammalian subject receiving antibiotic therapy and at risk of developing a Clostridium-associated disease an effective amount of a compound, a pharmaceutically acceptable salt of a compound, or a composition as disclosed herein.

In another aspect, the present disclosure provides a method of preventing a Clostridium-associated disease in a mammalian subject, comprising administering to a mammalian subject at risk of developing a Clostridium-associated disease an effective amount of a compound, a pharmaceutically acceptable salt of a compound, or a composition thereof, wherein the compound is of the formula:

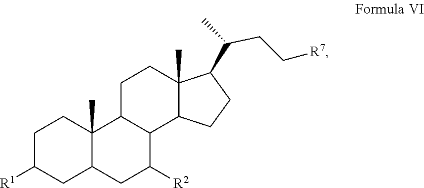

wherein: R¹ and R² are each independently selected from the group consisting of —H, —OR⁶, and —OC(O)R⁸; each R⁶ is independently selected from the group consisting of —H, a C1-C10 straight chain or branched chain alkyl or cycloalkyl group, —SO₃H, and a pharmaceutically acceptable salt of —SO₃H; R⁷ represents —C(O)OR⁸ or —C(O)N(R⁸)₂; and each R⁸ independently represents —H or a C1-C10 straight chain or branched chain alkyl or cycloalkyl group; with the proviso that one or both of R¹ and R² represent —OSO₃H or a pharmaceutically acceptable salt of —OSO₃H.

In another aspect, the present disclosure provides a method of treating a Clostridium-associated disease in a mammalian subject, comprising administering to a mammalian subject having a Clostridium-associated disease an effective amount of a compound, a pharmaceutically acceptable salt of a compound, or a composition thereof, wherein the compound is of the formula:

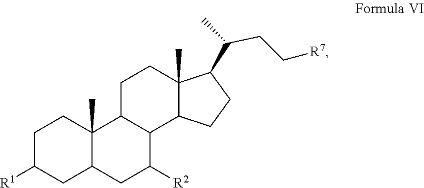

wherein: R¹ and R² are each independently selected from the group consisting of —H, —OR⁶, and —OC(O)R⁸; each R⁶ is independently selected from the group consisting of —H, a C1-C10 straight chain or branched chain alkyl or cycloalkyl group, —SO₃H, and a pharmaceutically acceptable salt of —SO₃H; R⁷ represents —C(O)OR⁸ or —C(O)N(R⁸)₂; and each R⁸ independently represents —H or a C1-C10 straight chain or branched chain alkyl or cycloalkyl group; with the proviso that one or both of R¹ and R² represent —OSO₃H or a pharmaceutically acceptable salt of —OSO₃H.

In another aspect, the present disclosure provides a method of reducing the risk of developing a Clostridium-associated disease in a mammalian subject receiving antibiotic therapy, comprising administering to a mammalian subject receiving antibiotic therapy and at risk of developing a Clostridium-associated disease an effective amount of a compound, a pharmaceutically acceptable salt of a compound, or a composition thereof, wherein the compound is of the formula:

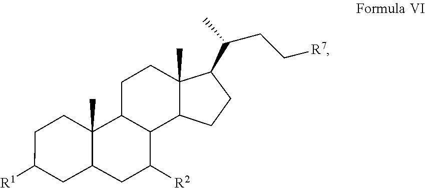

wherein: R¹ and R² are each independently selected from the group consisting of —H, —OR⁶, and —OC(O)R⁸; each R⁶ is independently selected from the group consisting of —H, a C1-C10 straight chain or branched chain alkyl or cycloalkyl group, —SO₃H, and a pharmaceutically acceptable salt of —SO₃H; R⁷ represents —C(O)OR⁸ or —C(O)N(R⁸)₂; and each R⁸ independently represents —H or a C1-C10 straight chain or branched chain alkyl or cycloalkyl group; with the proviso that one or both of R¹ and R² represent —OSO₃H or a pharmaceutically acceptable salt of —OSO₃H.

The compounds described herein have been designed to be stable to chemical and biological hydrolysis or modification. In some embodiments, the compounds described herein have been designed to avoid uptake into enterohepatic circulation. In some embodiments, the compounds disclosed herein also include a permanently charged sulfate group that may tend to inhibit passive absorption of the compound from the intestine into enterohepatic circulation.

The International Union of Pure and Applied Chemistry (IUPAC) numbering system used for steroids is used herein for denoting ring positions:

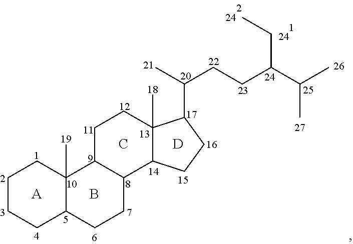

In one embodiment, the compounds disclosed herein include heterocyclic-containing groups at C23. In certain embodiments, compounds with heterocyclic-containing groups at C23 can be more stable to hydrolysis than, for example, an ester functional group. In certain embodiments, the compounds disclosed herein that include heterocyclic-containing groups at C23 can decrease the active transport of the compounds from the intestine.

In another embodiment, the compounds disclosed herein include oxygen substitutions at C6 (e.g., a hydroxyl, an ether group, or an alkanoate group). In certain embodiments, compounds with oxygen substitution at C6 may have lower affinity and transport rates in ileal transport systems. See, for example, Kramer et al., Journal of Lipid Research, 1999, 40:1604-1617.

In another embodiment, the compounds disclosed herein can include other groups or constituents at the C3 and/or C7 positions. For example, ether derivatives, carbonyl containing derivatives, F, and/or straight chain or branched chain alkyl or cycloalkyl groups (e.g., a methyl group) can be at the C3 and/or C7 positions. In certain embodiments, the ether derivatives, carbonyl containing derivatives, F, and/or straight chain or branched chain alkyl or cycloalkyl groups at the C3 and/or C7 positions may inhibit bacterial dehydroxylation of the C7 alcohol group, thereby preventing modification of the parent compounds.

In some embodiments, the compounds disclosed herein also include a permanently charged sulfate group at C7. In certain embodiments, the permanently charged sulfate group may tend to inhibit passive absorption of the compound from the intestine.

In some embodiments, the compounds disclosed herein can inhibit germination of Clostridium difficile spores at micromolar (μM) concentrations when incubated with spores in the presence of a known germinant.

### Definitions

The compounds of the present disclosure may exist in particular geometric or stereoisomeric forms. The present disclosure contemplates all such compounds, including cis- and trans-isomers, R- and S-enantiomers, diastereomers, (D)-isomers, (L)-isomers, the racemic mixtures thereof, and other mixtures thereof, as falling within the scope of the disclosure.

Diastereomers specifically include epimers, which are diastereomers that differ in configuration of only one stereogenic center.

Where an isomer/enantiomer or epimer is preferred, it may, in some embodiments, be provided substantially free of the corresponding enantiomer or epimer, and may also be referred to as “optically enriched.” “Optically enriched,” as used herein, means that the compound is made up of a significantly greater proportion of one enantiomer or epimer. In certain embodiments the compound of the present disclosure is made up of at least about 90% by weight of a preferred enantiomer or epimer. In other embodiments the compound is made up of at least about 95%, 98%, or 99% by weight of a preferred enantiomer or epimer. Preferred enantiomers or epimers may be isolated from racemic mixtures by any method known to those skilled in the art, including chiral high pressure liquid chromatography (HPLC) and the formation and crystallization of chiral salts or prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); Wilen, Tables of Resolving Agents and Optical Resolutions p. 268 (E. L. Eliel, Ed., Univ. of Notre Dame Press, Notre Dame, Ind. 1972).

As used herein, the term “organic group” is used for the purpose of this disclosure to mean a hydrocarbon group that is classified as an aliphatic group, cyclic group, or combination of aliphatic and cyclic groups (e.g., alkaryl and aralkyl groups). In the context of the present disclosure, suitable organic groups for compounds of this disclosure are those that do not interfere with the prevention, treatment, and/or reduction of risk for developing a Clostridium-associated disease. In the context of the present disclosure, the term “aliphatic group” means a saturated or unsaturated linear or branched hydrocarbon group. This term is used to encompass alkyl, alkenyl, and alkynyl groups, for example. The term “alkyl group” means a saturated linear or branched monovalent hydrocarbon group including, for example, methyl, ethyl, n-propyl, isopropyl, tert-butyl, amyl, heptyl, and the like. The term “alkenyl group” means an unsaturated, linear or branched monovalent hydrocarbon group with one or more olefinically unsaturated groups (i.e., carbon-carbon double bonds), such as a vinyl group. The term “alkynyl group” means an unsaturated, linear or branched monovalent hydrocarbon group with one or more carbon-carbon triple bonds. The term “cyclic group” means a closed ring hydrocarbon group that is classified as an alicyclic group, aromatic group, or heterocyclic group. The term “alicyclic group” means a cyclic hydrocarbon group having properties resembling those of aliphatic groups. The term “aromatic group” or “aryl group” means a mono- or polynuclear aromatic hydrocarbon group. The term “heterocyclic group” means a closed ring hydrocarbon in which one or more of the atoms in the ring is an element other than carbon (e.g., nitrogen, oxygen, sulfur, etc.).

As a means of simplifying the discussion and the recitation of certain terminology used throughout this application, the terms “group” and “moiety” are used to differentiate between chemical species that allow for substitution or that may be substituted and those that do not so allow for substitution or may not be so substituted. Thus, when the term “group” is used to describe a chemical substituent, the described chemical material includes the unsubstituted group and that group with nonperoxidic O, N, S, Si, or F atoms, for example, in the chain as well as carbonyl groups or other conventional substituents. Where the term “moiety” is used to describe a chemical compound or substituent, only an unsubstituted chemical material is intended to be included. For example, the phrase “alkyl group” is intended to include not only pure open chain saturated hydrocarbon alkyl substituents, such as methyl, ethyl, propyl, tert-butyl, and the like, but also alkyl substituents bearing further substituents known in the art, such as hydroxy, alkoxy, alkylsulfonyl, halogen atoms, cyano, nitro, amino, carboxyl, etc. Thus, “alkyl group” includes ether groups, haloalkyls, nitroalkyls, carboxyalkyls, hydroxyalkyls, sulfoalkyls, etc. On the other hand, the phrase “alkyl moiety” is limited to the inclusion of only pure open chain saturated hydrocarbon alkyl substituents, such as methyl, ethyl, propyl, tert-butyl, and the like.

The terms “comprises” and variations thereof do not have a limiting meaning where these terms appear in the description and claims.

As used herein, “a,” “an,” “the,” “at least one,” and “one or more” are used interchangeably.

Also herein, the recitations of numerical ranges by endpoints include all numbers subsumed within that range (e.g., 1 to 5 includes 1, 1.5, 2, 2.75, 3, 3.80, 4, 5, etc.).

The above brief description of various embodiments of the present disclosure is not intended to describe each embodiment or every implementation of the present disclosure. Rather, a more complete understanding of the disclosure will become apparent and appreciated by reference to the following description and claims in view of the accompanying drawings. Further, it is to be understood that other embodiments may be utilized and structural changes may be made without departing from the scope of the present disclosure.

### BRIEF DESCRIPTION OF THE DRAWINGS

FIGS. 1 and 2 are graphical representations illustrating changes in optical density over time indicative of spore germination in the presence of exemplary embodiments of compounds of Formula I at various concentrations, as described in Examples 1 and 2, respectively.

FIGS. 3-7 are graphical representations illustrating changes in optical density over time indicative of spore germination in the presence of exemplary comparative compounds at various concentrations, as described in Comparative Examples 1-5, respectively.

FIG. 8 is a graphical representation illustrating changes in optical density over time indicative of spore germination in the presence of an exemplary embodiment of a compound of Formula II at various concentrations, as described in Example 3.

FIGS. 9-10 are graphical representations illustrating changes in optical density over time indicative of spore germination in the presence of exemplary comparative compounds at various concentrations, as described in Comparative Example 6.

FIGS. 11-12 are graphical representations illustrating changes in optical density over time indicative of spore germination in the presence of exemplary embodiments of compounds of Formula I at various concentrations, as described in Example 4.

FIG. 13 is a graphical representation illustrating changes in optical density over time indicative of spore germination in the presence of an exemplary embodiment of a compound of Formula I at various concentrations, as described in Example 5.

FIGS. 14-15 and 16-17 are graphical representations illustrating changes in optical density over time indicative of spore germination in the presence of exemplary comparative compounds at various concentrations, as described in Comparative Examples 7 and 8, respectively.

FIGS. 18-20 are graphical representations illustrating changes in optical density over time indicative of spore germination in the presence of exemplary comparative compounds at various concentrations, as described in Comparative Examples 10, 11, and 14, respectively.

FIGS. 21 and 22 are graphical representations illustrating changes in optical density over time indicative of spore germination in the presence of exemplary embodiments of compounds of Formula III at various concentrations, as described in Example 6.

FIGS. 23 and 24 are graphical representations illustrating changes in optical density over time indicative of spore germination in the presence of exemplary embodiments of compounds of Formula I at various concentrations, as described in Examples 7 and 9, respectively.

FIG. 25 is a graphical representation illustrating changes in optical density over time indicative of spore germination in the presence of an exemplary comparative compound at various concentrations, as described in Comparative Example 20.

FIGS. 26-30 are graphical representations illustrating changes in optical density over time indicative of spore germination in the presence of exemplary embodiments of compounds of Formula I at various concentrations, as described in Examples 14-18, respectively.

### DETAILED DESCRIPTION OF ILLUSTRATIVE EMBODIMENTS

Sorg and Sonenshein explored ursodeoxycholic acid (UDCA) and 7 additional commercially available chenodeoxycholic acid (CDCA) analogs as inhibitors of spore germination in a UK1 C. difficile strain in an in vitro assay (Sorg et al., J. Bacteriol. 2010, 192:4983). Of the 8 compounds tested, five contained carboxylic acid moieties (—COOH) at the C23 position. The remaining three compounds had a methyl ester (—COOCH₃) at C23. See, also, U.S. Patent Application Pub. No. 2011/0280847 A1 (Sorg et al.)).

The Abel-Santos laboratory has also investigated bile acid analogs as potential inhibitors in C. difficile strain 630 (Howerton et al., J. Bacteriol. 2011, 193:274). Most of the compounds reported by the Abel-Santos laboratory are derived from cholic acid, which contains a C12 hydroxyl group. Modifications at the C23 position include amides with a linear one or two carbon chain that is substituted with a carboxylic acid, sulfonate (—SO₃H), or amide (—CONH₂). There are three other compounds described that include an aryl amide with a sulfonate substitution at the ortho-, para-, and meta-positions of the benzene ring (—CONH(C₆H₄)SO₃H) at C23.

However, none of these analogs were strong candidates as therapeutics for recurrent C. difficile infection, because esters, acetates, and amides are likely to be cleaved by chemical hydrolysis in the stomach or by various esterases and amidases in the body. Additionally, some of the C23 amides described (—CONHCH₂CH₂SO₃H) may be actively transported out of the intestine and enter enterohepatic circulation. Furthermore, one of these compounds, CamSA (Howerton et al., J. Bacteriol. 2011, 193:274) showed very little efficacy at inhibiting the germination of a highly virulent NAP1 strain of C. difficile, suggesting the need for more efficacious compounds.

Steer et al. have used sulfated versions of UDCA to prevent uptake of the bile acid from the colon into the enterohepatic system in rats (Rodrigues et al., Gastroenterology 1995, 109:1835). The sulfates were included at the C3, C7, and C3/C7 positions of UDCA, and they disclosed that the C7-sulfated UDCA was poorly absorbed from the colon of rats. See, also, PCT International Pub. No. WO 97/18816 A2 (Setchell et al.).

The use of UDCA to inhibit Clostridium difficile spore germination in a pouchitis case has also been reported (Weingarden et al., J Clin Gastroenterol. 2016 50:624-30).

In one aspect, the present disclosure provides compounds and compositions for preventing, treating, and/or reducing the risk of developing a Clostridium-associated disease in a mammalian subject.

In certain embodiments, cholic and other bile acid dervivatives (other than currently approved ursodeoxycholic acid) are disclosed herein. In certain embodiments, the cholic and other bile acid dervivatives disclosed herein may remain stable in the strongly acidic chemical environment of the stomach, avoid reuptake into the enterohepatic circulation, and maintain resistance to 7α-dehydroxylation by colonic flora. In certain embodiments, these compounds may be antigerminants, and may prevent Clostridium difficile spore germination. In certain embodiments, the compounds disclosed herein may prevent Recurrent Clostridium difficile infection in patients that do not respond to fecal microbial transplantation (FMT) therapy. In certain embodiments, the compounds disclosed herein can be used as a supplement or augment to FMT.

In certain embodiments, the compounds disclosed herein can act as replacements for secondary bile acids or their analogues that inhibit Clostridium difficile.

In certain embodiments, the compounds disclosed herein can be used in an antibiotic-free therapeutic strategy that does not require FMT. Because germination in the human colon is a recognized step in the pathogenesis of infection with Clostridium difficile, these bile acid drivatives may be useful pharmaceuticals capable of treating this disease, particularly in patients who have failed multiple rounds of recommended antibiotic therapy.

In certain embodiments, the use of compounds disclosed herein in a non-antibiotic alternative to FMT therapy can be advantageous by not killing useful and/or necessary microorganisms.

In certain embodiments, the compounds disclosed herein can be used in combination with antibiotics to reduce the likelihood of initial or recurrent Clostridium difficile infection.

In one embodiment, a compound for preventing, treating, and/or reducing the risk of developing a Clostridium-associated disease in a mammalian subject is of the formula:

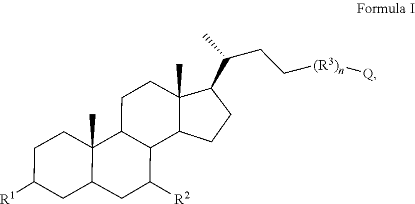

wherein: R¹ and R² are each independently selected from the group consisting of —H, —OR⁶, and —OC(O)R⁸; each R⁶ is independently selected from the group consisting of —H, a C1-C10 straight chain or branched chain alkyl or cycloalkyl group, —SO₃H, and a pharmaceutically acceptable salt of —SO₃H; R⁸ represents —H or a C1-C10 straight chain or branched chain alkyl or cycloalkyl group; R³ represents —C(O)— or a C1-C10 straight chain or branched chain alkylene or cycloalkylene group; n=0 or 1; and Q represents a heterocyclic ring, with the proviso that if R² is H, then Q does not represent a tetrazole.

In another embodiment, a compound for preventing, treating, and/or reducing the risk of developing a Clostridium-associated disease in a mammalian subject is of the formula:

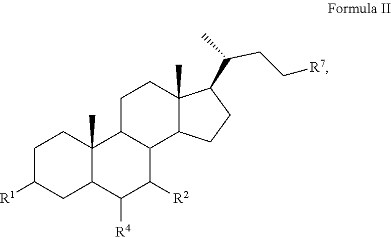

wherein: R¹ and R² are each independently selected from the group consisting of —H, —OR⁶, and —OC(O)R⁸; R⁴ represents —OR⁸ or —OC(O)R⁸; each R⁶ is independently selected from the group consisting of —H, a C1-C10 straight chain or branched chain alkyl or cycloalkyl group, —SO₃H, and a pharmaceutically acceptable salt of —SO₃H; R⁷ represents —C(O)OR⁸ or —(R³)ₙ-Q; each R⁸ independently represents —H or a C1-C10 straight chain or branched chain alkyl or cycloalkyl group; R³ represents —C(O)— or a C1-C10 straight chain or branched chain alkylene or cycloalkylene group; n=0 or 1; and Q represents a heterocyclic ring.

In another embodiment, a compound for preventing, treating, and/or reducing the risk of developing a Clostridium-associated disease in a mammalian subject is of the formula:

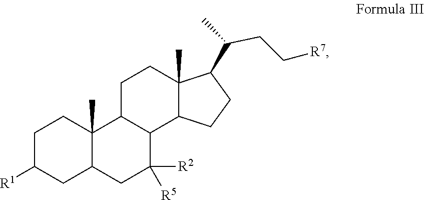

wherein: R¹ represents H, —OR⁶, or —OC(O)R⁸; R² represents —OR⁶ or —OC(O)R⁶; R⁵ represents F or a C1-C10 straight chain or branched chain alkyl or cycloalkyl group; each R⁶ is independently selected from the group consisting of —H, a C1-C10 straight chain or branched chain alkyl or cycloalkyl group, —SO₃H, and a pharmaceutically acceptable salt of —SO₃H; R⁷ represents —C(O)OR⁸ or —(R³)ₙ-Q; each R⁸ independently represents —H or a C1-C10 straight chain or branched chain alkyl or cycloalkyl group; R³ represents —C(O)— or a C1-C10 straight chain or branched chain alkylene or cycloalkylene group; n=0 or 1; and Q represents a heterocyclic ring. In certain embodiments, R⁵ is methyl.

In another embodiment, a compound for preventing, treating, and/or reducing the risk of developing a Clostridium-associated disease in a mammalian subject is of the formula:

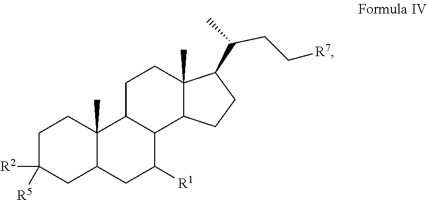

wherein: R¹ represents H, —OR⁶, or —OC(O)R⁸; R² represents —OR⁶ or —OC(O)R⁶; R⁵ represents F or a C1-C10 straight chain or branched chain alkyl or cycloalkyl group; each R⁶ is independently selected from the group consisting of —H, a C1-C10 straight chain or branched chain alkyl or cycloalkyl group, —SO₃H, and a pharmaceutically acceptable salt of —SO₃H; R⁷ represents —C(O)OR⁸ or —(R³)ₙ-Q; each R⁸ independently represents —H or a C1-C10 straight chain or branched chain alkyl or cycloalkyl group; R³ represents —C(O)— or a C1-C10 straight chain or branched chain alkylene or cycloalkylene group; n=0 or 1; and Q represents a heterocyclic ring. In certain embodiments, R⁵ is methyl.

In certain embodiments, compounds of Formula I, II, III, and/or IV can have a heterocyclic ring that is a three to seven-membered ring including 1 to 6 heteroatoms. In some certain embodiments, compounds of Formula I, II, III, and/or IV can have a heterocyclic ring that is a five or six-membered ring including 1 to 4 heteroatoms. In some certain embodiments, each of the heteroatoms are independently selected from the group consisting of N, O, and S.

In certain embodiments, compounds of Formula I, II, III, and/or IV can include a wide variety of heterocyclic rings. Exemplary heterocyclic rings include, but are not limited to, pyrrolidines, oxazolines, tetrazoles, oxadiazoles, imidazoles, trizoles, oxazoles, pyrazoles, thiazoles, isothiazoles, isoxazoles, piperidines, piperazines, pyridines, morpholines, pyrimidine, and combinations thereof. Exemplary heterocylic rings can include, but are not limited to, structures selected from the group consisting of:

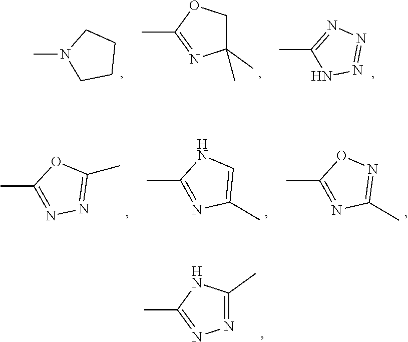

and combinations thereof.

In another embodiment, a compound for preventing, treating, and/or reducing the risk of developing a Clostridium-associated disease in a mammalian subject is of the formula:

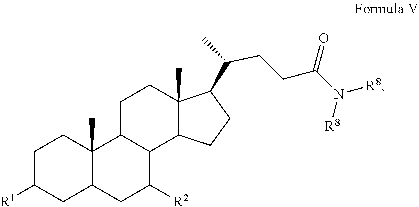

wherein: R¹ and R² are each independently selected from the group consisting of —H, —OR⁶, and —OC(O)R⁸; each R⁶ is independently selected from the group consisting of —H, a C1-C10 straight chain or branched chain alkyl or cycloalkyl group, —SO₃H, and a pharmaceutically acceptable salt of —SO₃H; and each R⁸ independently represents —H or a C1-C10 straight chain or branched chain alkyl or cycloalkyl group; with the proviso that one or both of R¹ and R² represent —OSO₃H or a pharmaceutically acceptable salt of —OSO₃H. In certain embodiments, the present disclosure further provides pharmaceutically acceptable salts of a compound of Formula V. In certain embodiments, the present disclosure further provides compositions that include a compound of Formula V and/or a pharmaceutically acceptable salt thereof.

For embodiments in which a compound of Formula I, II, III, IV, and/or V include a basic or acidic group, the compound may be in the form of a pharmaceutically acceptable salt.

Pharmaceutically Acceptable Salts

As set out herein, certain embodiments of the present compounds may contain a basic functional group, such as a sulfate, amino or alkylamino, and are, thus, capable of forming pharmaceutically-acceptable salts with pharmaceutically-acceptable acids. The term “pharmaceutically-acceptable salts” in this respect refers to the relatively non-toxic, inorganic and organic acid addition salts of compounds of the present disclosure. These salts can be prepared in situ in the administration vehicle or the dosage form manufacturing process, or by separately reacting a purified compound of the disclosure in its free base form with a suitable organic or inorganic acid, and isolating the salt thus formed during subsequent purification.

Representative salts include the hydrobromide, hydrochloride, sulfate, bisulfate, phosphate, phosphonate, nitrate, acetate, valerate, oleate, palmitate, stearate, laurate, benzoate, lactate, tosylate, citrate, maleate, fumarate, succinate, tartrate, napthylate, mesylate, glucoheptonate, lactobionate, and laurylsulphonate salts and the like. See, for example, Berge et al., J. Pharm. Sci. 66:1-19 (1977).

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present disclosure may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present disclosure. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. See, for example, Berge et al., J. Pharm. Sci. 66:1-19 (1977).

In another aspect, the present disclosure further provides compositions that include one or more compounds of Formula I, II, III, IV, and/or V or pharmaceutically acceptable salts thereof. The compositions can include a wide variety of additional components in addition to the one or more compounds of Formula I, II, III, IV, and/or V or pharmaceutically acceptable salts thereof.

Exemplary additional components include, but are not limited to, vehicles, encapsulants, and/or adjuvants.

Drug Formulations

Compositions useful in the practice of this disclosure can be formulated as pharmaceutical compositions together with pharmaceutically acceptable carriers for parenteral administration or enteral administration or for topical or local administration. For example, the compositions useful in the practice of the disclosure can be administered as oral formulations in solid or liquid form, or as intravenous, intramuscular, subcutaneous, transdermal, or topical formulations. Oral formulations for local delivery are preferred.

The compounds and compositions are typically administered with pharmaceutically acceptable carriers. The term “pharmaceutically-acceptable carrier” as used herein means one or more compatible solid, or semi-solid or liquid fillers, diluents or encapsulating substances which are suitable for administration to a human or other mammal such as a dog, cat, horse, cow, sheep, or goat. The term “carrier” denotes an organic or inorganic ingredient, natural or synthetic, with which the active ingredient is combined to facilitate the application. The carriers are capable of being commingled with the preparations of the present disclosure, and with each other, in a manner such that there is no interaction which would substantially impair the desired pharmaceutical efficacy or stability. Carriers suitable for oral and rectal formulations can be found in Remington's Pharmaceutical Sciences, Mack Publishing Company, Easton, Pa.

Pharmaceutically acceptable carriers for oral administration include capsules, tablets, pills, powders, troches, and granules. In the case of solid dosage forms, the carrier can comprise at least one inert diluent such as sucrose, lactose or starch. Such carriers can also comprise, as is normal practice, additional substances other than diluents, e.g. lubricating agents such as magnesium stearate. In the case of capsules, tablets, troches and pills, the carrier can also comprise buffering agents. Carriers, such as tablets, capsules, pills, and granules, can be prepared with coatings on the surfaces of the tablets, pills or granules which control the timing and/or the location of release of the pharmaceutical compositions in the gastrointestinal tract. In some embodiments, the carriers also target the active compositions to particular regions of the gastrointestinal tract and even hold the active ingredients at particular regions, such as is known in the art. Alternatively, the coated compounds can be pressed into tablets, pills, or granules. Pharmaceutically acceptable carriers include liquid dosage forms for oral administration, e.g. emulsions, solutions, suspensions, syrups and elixirs containing inert diluents commonly used in the art, such as water. Besides such inert diluents, compositions can also include additional inactive components such as wetting agents, emulsifying and suspending agents, and sweetening and other flavoring agents.

The pharmaceutical preparations of the disclosure may be provided in particles. Particles as used herein means nano- or microparticles (or in some instances larger) which can consist in whole or in part of the peripheral opioid antagonists or the other therapeutic agent(s) as described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero order release, first order release, second order release, delayed release, sustained release, immediate release, and any combination thereof, etc. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain the antagonist in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described by Sawhney et al., Macromolecules, 26:581-587 (1993), the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

When used in its acid form, a compound of the present disclosure can be employed in the form of a pharmaceutically acceptable salt of the acid. Carriers such as solvents, water, buffers, alkanols, cyclodextrins and aralkanols can be used. Other auxiliary, non-toxic agents may be included, for example, polyethylene glycols or wetting agents.

The pharmaceutically acceptable carriers and compounds described in the present disclosure are formulated into unit dosage forms for administration to the patients. The dosage levels of active ingredients (i.e. compounds of the present disclosure) in the unit dosage may be varied so as to obtain an amount of active ingredient that is effective to achieve a therapeutic effect in accordance with the desired method of administration. The selected dosage level therefore mainly depends upon the nature of the active ingredient, the route of administration, and the desired duration of treatment. If desired, the unit dosage can be such that the daily requirement for an active compound is in one dose, or divided among multiple doses for administration, e.g. two to four times per day.

The pharmaceutical preparations of the disclosure, when used in alone or together with other agents including antibiotics, are administered in therapeutically effective amounts. A therapeutically effective amount will be that amount which establishes a level of the drug(s) effective for treating a subject, such as a human subject. An effective amount means that amount, alone or with multiple doses, necessary to achieve a desired biological effect. When administered to a subject, effective amounts will depend, of course, on the particular effect chosen as the end-point; the severity of the condition being treated; individual patient parameters including age, physical condition, and weight; concurrent treatment; frequency of treatment; and the mode of administration. These factors are well known to those of ordinary skill in the art and can be addressed with no more than routine experimentation.

In some embodiments, the compounds disclosed herein have been tested for efficacy in prevention of germination of Clostridium difficile spores in an in vitro model. Inhibition of taurocholic acid-mediated germination of several strains of Clostridium difficile, including the hypervirulent NAP1 strain, has been observed at less than or equal to 1 mM concentration, in vitro.

Accordingly, in another aspect, the present disclosure provides a method of preventing a Clostridium-associated disease in a mammalian subject, including administering to a mammalian subject at risk of developing a Clostridium-associated disease an effective amount of a compound, a pharmaceutically acceptable salt of a compound, or a composition as disclosed herein. In certain embodiments, the Clostridium-associated disease is a Clostridium difficile-associated disease or a Clostridium perfringens-associated disease. In certain embodiments, the C. difficile-associated disease is C. difficile colitis. In other certain embodiments, the C. difficile-associated disease is pseudomembranous colitis. In certain embodiment, the subject is a human.

In another aspect, the present disclosure provides a method of treating a Clostridium-associated disease in a mammalian subject, including administering to a mammalian subject having a Clostridium-associated disease an effective amount of a compound, a pharmaceutically acceptable salt of a compound, or a composition as disclosed herein. In certain embodiments, the Clostridium-associated disease is a Clostridium difficile-associated disease or a Clostridium perfringens-associated disease. In certain embodiments, the C. difficile-associated disease is C. difficile colitis. In other certain embodiments, the C. difficile-associated disease is pseudomembranous colitis. In certain embodiment, the subject is a human.

In another aspect, the present disclosure provides a method of reducing the risk of developing a Clostridium-associated disease in a mammalian subject receiving antibiotic therapy, including administering to a mammalian subject receiving antibiotic therapy and at risk of developing a Clostridium-associated disease an effective amount of a compound, a pharmaceutically acceptable salt of a compound, or a composition as disclosed herein. In certain embodiments, the Clostridium-associated disease is a Clostridium difficile-associated disease or a Clostridium perfringens-associated disease. In certain embodiments, the C. difficile-associated disease is C. difficile colitis. In other certain embodiments, the C. difficile-associated disease is pseudomembranous colitis. In certain embodiments, the subject is a human.

In another aspect, the present disclosure provides a method of preventing a Clostridium-associated disease in a mammalian subject, comprising administering to a mammalian subject at risk of developing a Clostridium-associated disease an effective amount of a compound, a pharmaceutically acceptable salt of a compound, or a composition thereof, wherein the compound is of the formula:

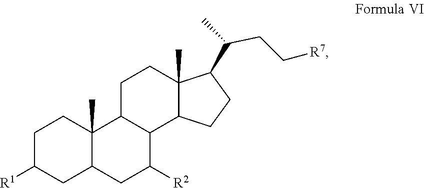

wherein: R¹ and R² are each independently selected from the group consisting of —H, —OR⁶, and —OC(O)R⁸; each R⁶ is independently selected from the group consisting of —H, a C1-C10 straight chain or branched chain alkyl or cycloalkyl group, —SO₃H, and a pharmaceutically acceptable salt of —SO₃H; R⁷ represents —C(O)OR⁸ or —C(O)N(R⁸)₂; and each R⁸ independently represents —H or a C1-C10 straight chain or branched chain alkyl or cycloalkyl group; with the proviso that one or both of R¹ and R² represent —OSO₃H or a pharmaceutically acceptable salt of —OSO₃H. In certain embodiments, the Clostridium-associated disease is a Clostridium difficile-associated disease or a Clostridium perfringens-associated disease. In certain embodiments, the C. difficile-associated disease is C. difficile colitis. In other certain embodiments, the C. difficile-associated disease is pseudomembranous colitis. In certain embodiments, the subject is a human.

In another aspect, the present disclosure provides a method of treating a Clostridium-associated disease in a mammalian subject, comprising administering to a mammalian subject having a Clostridium-associated disease an effective amount of a compound, a pharmaceutically acceptable salt of a compound, or a composition thereof, wherein the compound is of the formula:

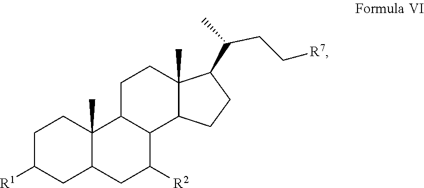

wherein: R¹ and R² are each independently selected from the group consisting of —H, —OR⁶, and —OC(O)R⁸; each R⁶ is independently selected from the group consisting of —H, a C1-C10 straight chain or branched chain alkyl or cycloalkyl group, —SO₃H, and a pharmaceutically acceptable salt of —SO₃H; R⁷ represents —C(O)OR⁸ or —C(O)N(R⁸)₂; and each R⁸ independently represents —H or a C1-C10 straight chain or branched chain alkyl or cycloalkyl group; with the proviso that one or both of R¹ and R² represent —OSO₃H or a pharmaceutically acceptable salt of —OSO₃H. In certain embodiments, the Clostridium-associated disease is a Clostridium difficile-associated disease or a Clostridium perfringens-associated disease. In certain embodiments, the C. difficile-associated disease is C. difficile colitis. In other certain embodiments, the C. difficile-associated disease is pseudomembranous colitis. In certain embodiments, the subject is a human.

In another aspect, the present disclosure provides a method of reducing the risk of developing a Clostridium-associated disease in a mammalian subject receiving antibiotic therapy, comprising administering to a mammalian subject receiving antibiotic therapy and at risk of developing a Clostridium-associated disease an effective amount of a compound, a pharmaceutically acceptable salt of a compound, or a composition thereof, wherein the compound is of the formula:

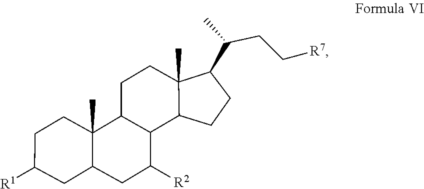

wherein: R¹ and R² are each independently selected from the group consisting of —H, —OR⁶, and —OC(O)R⁸; each R⁶ is independently selected from the group consisting of —H, a C1-C10 straight chain or branched chain alkyl or cycloalkyl group, —SO₃H, and a pharmaceutically acceptable salt of —SO₃H; R⁷ represents —C(O)OR⁸ or —C(O)N(R⁸)₂; and each R⁸ independently represents —H or a C1-C10 straight chain or branched chain alkyl or cycloalkyl group; with the proviso that one or both of R¹ and R² represent —OSO₃H or a pharmaceutically acceptable salt of —OSO₃H. In certain embodiments, the Clostridium-associated disease is a Clostridium difficile-associated disease or a Clostridium perfringens-associated disease. In certain embodiments, the C. difficile-associated disease is C. difficile colitis. In other certain embodiments, the C. difficile-associated disease is pseudomembranous colitis. In certain embodiments, the subject is a human.

Routes of Delivery

In general, the compounds and compositions employed in the methods of the disclosure can be administered enterally. In one embodiment compounds and compositions employed in the methods of the disclosure can be administered orally.

In one embodiment compounds and compositions employed in the methods of the disclosure can be administered rectally.

The active ingredient may be administered once, or it can be divided into a number of smaller doses to be administered at intervals of time. It is understood that the precise dosage and duration of treatment is a function of the disease being treated and may be determined empirically using known testing protocols or by extrapolation from in vivo or in vitro test data. For example, the dosage and duration of treatment can be determined by extrapolation from in vivo data obtained using one or more animal models of C. difficile-associated disease. It is to be noted that concentrations and dosage values may also vary with the severity of the condition to be alleviated. It is to be further understood that for any particular subject, specific dosage regimens can be adjusted over time according to the individual need and the professional judgment of the person administering or supervising the administration of the compositions, and that the concentration ranges set forth herein are exemplary only and are not intended to limit the scope or practice of the claimed disclosure.

If oral administration is desired, the active compound should be provided in a composition that protects it from the acidic environment of the stomach. For example, the composition can be formulated in an enteric coating that maintains its integrity in the stomach and releases the active compound in the intestine. The composition may also be formulated in combination with an antacid or other ingredient that is protective against the acidic environment of the stomach.

Oral compositions will generally include an inert diluent or an edible carrier and may be compressed into tablets or enclosed in gelatin capsules. For the purpose of oral therapeutic administration, the active compound or compounds can be incorporated with excipients and used in the form of tablets, capsules, or troches. Pharmaceutically compatible binding agents and adjuvant materials can be included as part of the composition.

The tablets, pills, capsules, troches, and the like can contain any of the following ingredients or compounds of a similar nature: a binder such as, but not limited to, gum tragacanth, acacia, corn starch, or gelatin; an excipient such as microcrystalline cellulose, starch, or lactose; a disintegrating agent such as, but not limited to, alginic acid and corn starch; a lubricant such as, but not limited to, magnesium stearate; a gildant, such as, but not limited to, colloidal silicon dioxide; a sweetening agent such as sucrose or saccharin; and a flavoring agent such as peppermint, methyl salicylate, or fruit flavoring.

When the dosage unit form is a capsule, it can contain, in addition to material of the above type, a liquid carrier such as a fatty oil. In addition, dosage unit forms can contain various other materials, which modify the physical form of the dosage unit, for example, coatings of sugar and other enteric agents. The compounds can also be administered as a component of an elixir, suspension, syrup, wafer, chewing gum or the like. A syrup may contain, in addition to the active compounds, sucrose as a sweetening agent and certain preservatives, dyes and colorings, and flavors.

The active materials can also be mixed with other active materials that do not impair the desired action, or with materials that supplement the desired action.

Enteric coated tablets are well known to those skilled in the art. In addition, capsules filled with small spheres each coated to protect from the acidic stomach, are also well known to those skilled in the art.

The oral dosage forms generally are administered to the patient one to four times daily. It is preferred that the compounds employed in the methods of the disclosure be administered either three or fewer times a day, more preferably once or twice daily.

When administered orally, an administered amount therapeutically effective to inhibit spore germination or to inhibit growth is from about 1 mg/kg body weight/day to about 100 mg/kg body weight/day. In one embodiment the oral dosage is from about 1 mg/kg body weight/day to about 50 mg/kg body weight/day.

In one embodiment the oral dosage is from about 5 mg/kg body weight/day to about 50 mg/kg body weight/day. It is understood that while a patient may be started at one dose, that dose may be varied over time as the patient's condition changes.

Pharmaceutical formulations adapted for “rectal administration” may be presented as suppositories or as enemas. These formulations which are presented as suppositories can be prepared by mixing the drug with a suitable non-irritating excipient which is solid at ordinary temperatures but liquid at the rectal temperatures and will therefore melt in the rectum to release the drug. Such materials are cocoa butter and polyethylene glycols.

As used herein, a “subject” is defined as a mammal and illustratively includes humans, non-human primates, horses, goats, cows, sheep, pigs, dogs, cats, and rodents. In one embodiment a subject is a human.

Evaluation of Biological Activity of Compounds

Some of the key steps in spore germination are depolymerization of peptidoglycan in the spore cortex followed by hydration of the spore core, which can be observed as a phase change from bright to dark by phase-contrast microscopy. This change also can be observed by measuring the decrease in optical density of the spore suspension at 600 nm (OD₆₀₀) over time. An optical density assay was used as an initial screen to identify the most potent bile acid analogs. These compounds were then further evaluated using a phase-contrast microscopy assay to verify the initial findings and obtain more quantitative data.

Spores from a NAP1 strain of C. difficile were isolated from patient samples by previously reported methods. In the optical density assay, spores were incubated for 20 minutes on brain-heart infusion medium supplemented with 0.5% yeast extract and 0.1% 1-cysteine (BHIS) along with 2000 μM taurocholic acid (TCA), a known germinant, under anaerobic conditions in the presence of the bile acid analogs. As a control for this experiment, spores were incubated with 2000 μM TCA for 20 minutes in the absence of test compounds, which resulted in a maximum change in OD₆₀₀ to approximately 60% of the initial value. Compounds showing significant potency at inhibiting germination in the optical density were retested, with similar results obtained between runs.

In the phase-contrast spore count assay, NAP1 spores were plated with either dimethyl sulfoxide (DMSO) as a control or the test compounds at 10 μM. The number of spores and germinated cells were counted at time zero (t₀). The spores were then exposed to 2000 μM TCA for 20 minutes and the number of spores and germinated cells were counted at t₂₀ for both the DMSO control and the test compound plates. Each experiment was performed in triplicate and the percentage of germinated cells at t₀ and t₂₀ determined. In the DMSO control samples, the percent of germinated spores increased from approximately 20% to 75% of the total number of cells after 20 minutes.

Results of Optical Density Assay Spores were incubated in BHIS supplemented with 2000 μM of the germinant TCA with varying concentrations of the bile acid analogs. Compounds were divided into three categories based on the lowest concentration at which complete inhibition of spore germination was observed (defined as a relative OD₆₀₀ reading after 20 min. greater than or equal to 0.95). Good inhibitors were effective at 50 μM or less, moderate inhibitors were effective at 100-500 μM, and poor inhibitors were effective at concentrations ≥1000 μM.

The present disclosure is illustrated by the following examples. It is to be understood that the particular examples, materials, amounts, and procedures are to be interpreted broadly in accordance with the scope and spirit of the disclosure as set forth herein.

### EXAMPLES AND COMPARATIVE EXAMPLES

The following Examples and Comparative Examples describe preparation and characterization of the indicated compounds.

NMR spectra were recorded using a Bruker 400 spectrometer. ¹H NMR data are reported as follows: chemical shift in parts per million downfield of tetramethylsilane (TMS) or relative to the residual solvent peak, multiplicity (s=singlet, bs=broad singlet, d=doublet, t=triplet, q=quartet, quint=quintet and m=multiplet), coupling constant (Hz), and integrated value. Unless otherwise specified, all materials, reagents, and solvents were obtained from commercial suppliers and were used without further purification. The progress of a synthetic procedure was monitored, where possible, by thin layer chromatography (TLC) and the compounds of interest were visualized using PMA/Ce(SO₄)₂ stain. TLC was conducted on silica gel 250 μm, F254 plates. Flash column chromatography was performed using Teledyne-Isco Combiflash Rf+Purlon with Redisep Rf silica gel columns. The purity of the compounds evaluated in the phase-contrast spore count assay was determined to be >95% by LC-MS/UV/ELSD analysis.

Isolation and characterization of C. difficile isolates was performed as previously reported (Weingarden et al., PLoS One 2016, 11, e0147210).

C. difficile Spore Preparation

C. difficile cells from frozen stocks were inoculated into cycloserine-cefoxitin-fructose broth (CCFB medium) and grown anaerobically at 37° C. for 48 hours. Cultures were plated onto brain-heart infusion medium supplemented with 0.5% yeast extract and 0.1% 1-cysteine (BHIS), and grown for 7 days at 37° C. under anaerobic conditions. Colonies from each plate were scraped into 1 mL of ice-cold water and incubated at 4° C. overnight to release spores. A 3 mL aliquot of this suspension was loaded onto 10 mL of 50% (w/v) sucrose in a 15 mL conical tube, and centrifuged in a swinging bucket rotor at 3200×g for 20 minutes at 4° C. Sucrose and vegetative cells above the spore pellet were removed, and the pellet was washed 5 times in ice-cold water to remove remaining sucrose. Spores were examined under phase-contrast microscopy to determine purity. Spore samples with >99% purity (<1% vegetative cells) were stored at 4° C. and used in the studies.

C. difficile Spore Germination Assays

Optical Density Assay

BHIS was spiked with TCA to a final concentration of 2000 μM to test compounds for inhibition of germination. Either 130 μL of BHIS media (negative control) or the BHIS with 2000 μM TCA was added to separate wells of a 96 well plate in triplicate. The test compound (10 μL) in solvent (typically DMSO or DMSO/ethanol) was added to both the BHIS and BHIS with TCA in triplicate. In addition, BHIS with 2000 μM TCA (positive control) was amended, in triplicate, with solvent (DMSO or DMSO/ethanol). BHIS with DMSO or DMSO/ethanol and filter sterilized water were also run in duplicate as a negative control. The 96 well plates containing the solutions were reduced in an anaerobic chamber for 2-3 hours. Spores, at approximately a 0.5 McFarland standard concentration, were heated to 65° C. for 30 minutes before being inoculated into the test media, with or without spiked bile acid analogs solutions, within an anaerobic bag flushed and filled with nitrogen gas. The OD₆₀₀ was measured initially (OD₆₀₀(t₀)), and once every minute for 20 minutes (OD₆₀₀(t)) using an EL808 Microplate Reader (Biotek Instruments, Inc., Winooski, Vt.). Relative OD₆₀₀ for each time point was calculated as OD₆₀₀(t)/OD₆₀₀(t₀).

A decrease in optical density to approximately 60% of the initial value after 20 minutes is indicative of complete spore germination in this assay. Compounds in the presence of spores and taurocholate (TCA) that prevented a decrease in OD₆₀₀ after 20 minutes were considered to be inhibitors of spore germination. In general, active inhibitors consisted of a variety of functional groups on the bile acid analogs including esters, amides, heterocycles, ethers, and sulfonates.

Microscope Spore Count Assay

Media and spore preparation for phase contrast assay was performed in the same method as outlined in the optical density assay above. To samples were immediately placed on ice, while the t₂₀ samples were incubated at room temperature for 20 minutes. Following the 20 minute incubation, the t₂₀ samples were also placed on ice to prevent further germination. 10 μl of each of the triplicate samples were removed and counted on a hemocytometer under a Plan 40/0.65 Ph2 lens for counts of spores and germinated cells. Experiments were performed in triplicate and average percentage of germination was determined.

### Example 1: Compound of Formula I Having the Structure

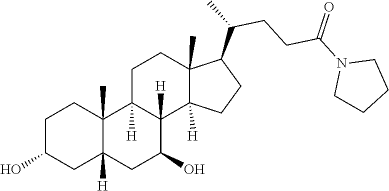

To a solution of UDCA (0.500 g, 1.27 mmol), triethylamine (0.0.53 mL, 3.8 mmol), and pyrrolidine (0.21 mL, 2.6 mmol) in dichloromethane (DCM) (13 mL) was added 1-[Bis(dimethylamino)methylene]-1H-1,2,3-triazolo[4,5-b]pyridinium 3-oxide hexafluorophosphate (HATU, 0.533 g, 1.40 mmol). The reaction mixture was stirred at room temperature for 16 hours. The reaction was quenched by the addition of saturated aqueous NaHCO₃ (6 mL) and the aqueous layer was extracted with DCM (3×5 mL). The combined organic layers were dried over MgSO₄, filtered, and concentrated. The crude material was purified by flash column chromatography on silica gel (2-5% methanol (MeOH) in ethyl acetate (EtOAc) over 35 minutes) to obtain KLS-1-015 (0.495 g, 87% yield) as an off-white foam.

¹H NMR (400 MHz, CDCl₃) δ 3.60 (ddt, J=15.5, 10.3, 4.8 Hz, 2H), 3.45 (dt, J=16.2, 6.8 Hz, 4H), 2.32 (ddd, J=15.5, 10.8, 5.3 Hz, 1H), 2.22-2.21 (m, 1H), 1.98 (dtd, J=18.4, 12.5, 11.7, 7.4 Hz, 4H), 1.90-1.75 (m, 6H), 1.74-1.65 (m, 2H), 1.64-1.56 (m, 2H), 1.48 (ddt, J=19.7, 14.2, 6.9 Hz, 8H), 1.41-1.32 (m, 2H), 1.32-1.21 (m, 2H), 1.21-0.98 (m, 4H), 0.96 (app. t, J=3.2 Hz, 6H), 0.70 (s, 3H).

¹³C NMR (100 MHz, CDCl₃) δ 172.2, 76.7, 71.5, 71.4, 55.7, 55.0, 46.6, 45.6, 43.8, 43.8, 42.4, 40.1, 39.2, 37.3, 36.8, 35.5, 34.9, 34.1, 31.7, 31.0, 30.3, 28.6, 26.9, 26.2, 24.4, 23.4, 21.2, 18.6.

FIG. 1 is a graphical representation illustrating changes in optical density over time indicative of spore germination in the presence of the compound described in Example 1 at various concentrations. The graph illustrates essentially complete inhibition of spore germination by KLS-1-015 at 0.1 mM.

### Example 2: Compound of Formula I Having the Structure

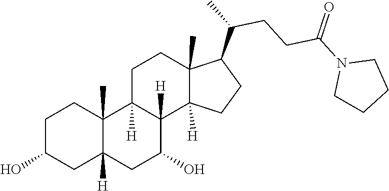

To a solution of CDCA (0.500 g, 1.27 mmol), triethylamine (0.53 mL, 3.0 mmol), and pyrrolidine (0.21 mL, 2.6 mmol) in DCM (13 mL) was added HATU (0.533 g, 1.40 mmol). The reaction mixture was stirred at room temperature for 5 hours. The reaction was quenched by the addition of water (12 mL) and the aqueous layer was extracted with DCM (3×5 mL). The combined organic layers were dried over MgSO₄, filtered, and concentrated. The crude material was purified by column chromatography on silica gel (2-5% MeOH in EtOAc over 35 minutes) to obtain KLS-1-016 (0.400 g, 71% yield) as a white solid.

¹H NMR (400 MHz, CDCl₃) δ 3.85 (bs, 1H), 3.43 (dt, J=17.6, 6.8 Hz, 5H), 2.30 (ddt, J=14.4, 8.7, 4.4 Hz, 1H), 2.21 (d, J=13.8 Hz, 1H), 2.14 (dt, J=15.2, 5.2 Hz, 1H), 2.02-1.89 (m, 5H), 1.87-1.78 (m, 5H), 1.76-1.66 (m, 3H), 1.54-1.44 (m, 4H), 1.43-1.30 (m, 6H), 1.30-1.19 (m, 3H), 1.18-1.05 (m, 2H), 1.00 (dd, J=14.3, 3.3 Hz, 1H), 0.95 (d, J=6.4 Hz, 3H), 0.91 (s, 3H), 0.67 (s, 3H).

¹³C NMR (100 MHz, CDCl₃) δ 172.2, 72.0, 68.5, 55.8, 50.5, 46.6, 45.6, 42.7, 41.5, 39.9, 39.7, 39.4, 35.5, 35.3, 35.0, 34.6, 32.8, 31.6, 30.9, 30.7, 28.2, 26.2, 24.4, 23.7, 22.8, 20.6, 18.5, 11.8.

HRMS (ESI): m/z calcd. C₂₈H₄₇NO₃Na (M+Na⁺) 468.3454, found 468.3454.

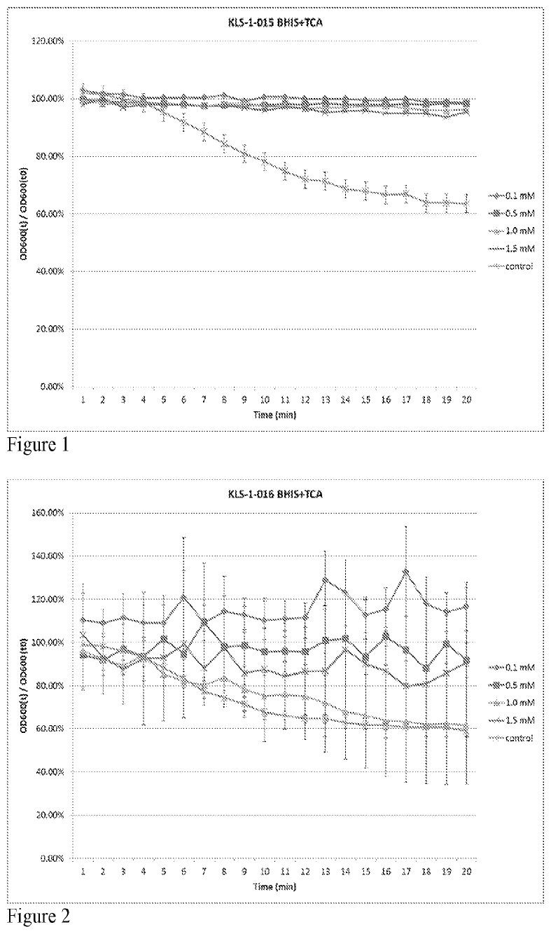

FIG. 2 is a graphical representation illustrating changes in optical density over time indicative of spore germination in the presence of the compound described in Example 2 at various concentrations. The graph illustrates essentially complete inhibition of spore germination by KLS-1-016 at 0.1 mM.

### Comparative Example 1: Compound Having the Structure

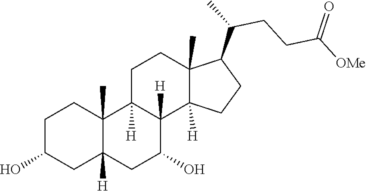

To a solution of CDCA (1.00 g, 2.55 mmol) in MeOH (26 mL) was added p-toluenesulfonic acid monohydrate (TsOH.H₂O, 0.048 g, 0.255 mmol) and the reaction stirred at room temperature for 24 hours. The reaction was quenched by the addition of NaHCO₃ (5 mL) and the solvent was removed by rotary evaporation. The residue was partitioned between NaHCO₃ and EtOAc, and the aqueous layer was extracted with EtOAc (3×15 mL). The combined organic layers were washed with NaHCO₃ (3×10 mL) and water (5 mL). The organic layer was dried over MgSO₄, filtered, and concentrated. The crude material was purified by flash column chromatography on silica gel (40% EtOAc in hexanes over 25 minutes) to obtain KLS-1-008 (0.929 g, 90% yield) as a white solid.

¹H NMR (400 MHz, CDCl₃) δ 3.78 (q, J=2.9 Hz, 1H), 3.59 (s, 3H), 3.39 (tt, J=11.2, 4.5 Hz, 1H), 2.28 (ddd, J=15.3, 10.1, 5.1 Hz, 1H), 2.22-2.06 (m, 2H), 1.96-1.86 (m, 2H), 1.86-1.80 (m, 1H), 1.80-1.70 (m, 3H), 1.70-1.51 (m, 3H), 1.49-1.37 (m, 4H), 1.36-1.17 (m, 9H), 1.17-0.98 (m, 3H), 0.93 (dd, J=14.2, 3.4 Hz, 1H), 0.86 (d, J=6.4 Hz, 3H), 0.84 (s, 3H), 0.59 (s, 3H).

¹³C NMR (100 MHz, CDCl₃) δ 174.7, 72.0, 68.5, 55.8, 51.5, 50.5, 42.7, 41.5, 39.9, 39.6, 39.4, 35.4, 35.3, 35.0, 34.6, 32.8, 31.0, 31.0, 30.7, 28.1, 23.7, 22.8, 20.6, 18.3, 11.8.

HRMS (ESI): m/z calcd. C₂₅H₄₂NO₄Na (M+Na⁺) 429.2981, found 429.2961.

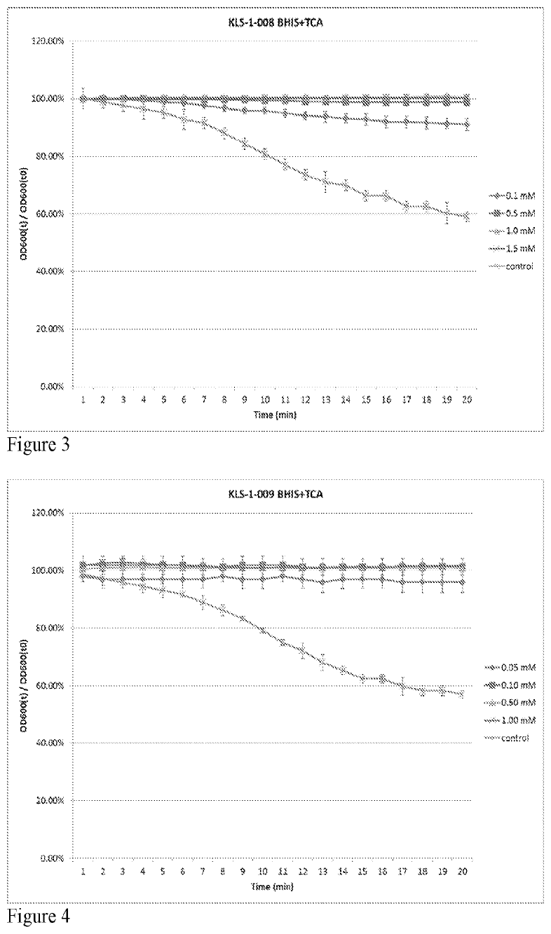

FIG. 3 is a graphical representation illustrating changes in optical density over time indicative of spore germination in the presence of an exemplary comparative compound as described in Comparative Example 1 at various concentrations. The graph illustrates essentially complete inhibition of spore germination by KLS-1-008 at 0.5 mM.

### Comparative Example 2: Compound Having the Structure

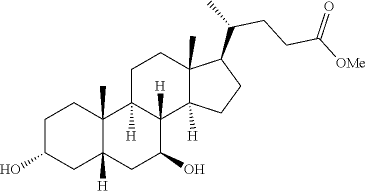

To a solution of UDCA (1 g, 2.55 mmol) in MeOH (26 mL) was added TsOH.H₂O (0.048 g, 0.255 mmol) and the reaction stirred at room temperature for 24 hours. The reaction was quenched by the addition of NaHCO₃ (5 mL) and the solvent was removed by rotary evaporation. The residue was partitioned between NaHCO₃ and EtOAc, and the aqueous layer was extracted with EtOAc (3×15 mL). The combined organic layers were washed with NaHCO₃ (3×10 mL), water (5 mL), dried over MgSO₄, filtered, and concentrated to obtain KLS-1-009 (0.933 g, 90% yield) as a white solid.

¹H NMR (400 MHz, CDCl₃) δ 3.67 (s, 3H), 3.60 (tt, J=10.4, 4.6 Hz, 2H), 2.37 (ddd, J=15.3, 10.1, 5.0 Hz, 1H), 2.23 (ddd, J=15.6, 9.5, 6.5 Hz, 1H), 2.01 (dt, J=12.5, 3.2 Hz, 1H), 1.95-1.88 (m, 1H), 1.86-1.75 (m, 4H), 1.72-1.65 (m, 2H), 1.65-1.57 (m, 2H), 1.57-1.50 (m, 2H), 1.51-1.40 (m, 6H), 1.40-1.21 (m, 5H), 1.20-0.98 (m, 3H), 0.96 (s, 3H), 0.94 (d, J=6.4 Hz, 3H), 0.69 (s, 3H).

¹³C NMR (100 MHz, CDCl₃) δ 174.7, 71.4, 71.3, 55.7, 54.9, 51.5, 43.8, 43.8, 42.4, 40.1, 39.2, 37.3, 36.8, 35.3, 34.9, 34.1, 31.1, 31.0, 30.3, 28.6, 26.9, 23.4, 21.2, 18.4, 12.1.

HRMS (ESI): m/z calcd. C₂₅H₄₂NO₄Na (M+Na⁺) 429.2981, found 429.2965.

FIG. 4 is a graphical representation illustrating changes in optical density over time indicative of spore germination in the presence of an exemplary comparative compound as described in Comparative Example 2 at various concentrations. The graph illustrates essentially complete inhibition of spore germination by KLS-1-009 at 0.1 mM.

### Comparative Example 3: Compound Having the Structure

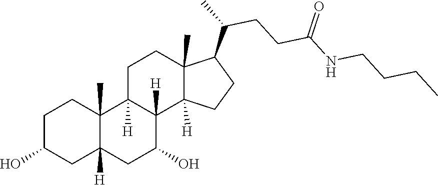

To a solution of CDCA (0.393 g, 1.00 mmol) in tetrahydrofuran (THF, 10 mL) was added 1,1′-carbonyldiimidazole (CDI, 0.243 g, 1.50 mmol) and the reaction stirred for 1 hour at room temperature. To the solution was added butan-1-amine (0.198 mL, 2.00 mmol) and the reaction stirred for 18 hours at room temperature. The reaction was quenched with NH₄Cl (10 mL). The aqueous and organic layers were separated and the aqueous layer was extracted with EtOAc (3×10 mL). The combined organic layers were washed with NH₄Cl (3×5 mL), water (5 mL), then dried over MgSO₄, filtered, and concentrated. The crude material was purified by flash column chromatography on silica gel (2-5% MeOH/EtOAc 35 minutes, then 10% MeOH/EtOAc flush) to obtain KLS-1-010 (0.083 g, 19% yield) as an off-white solid.

¹H NMR (400 MHz, CDCl₃) δ 5.58 (t, J=5.7 Hz, 1H), 3.84 (q, J=2.7 Hz, 1H), 3.45 (tt, J=11.0, 4.5 Hz, 1H), 3.23 (q, J=6.7 Hz, 2H), 2.28-2.13 (m, 2H), 2.10-2.01 (m, 1H), 2.02-1.86 (m, 4H), 1.87-1.75 (m, 3H), 1.75-1.58 (m, 3H), 1.58-1.22 (m, 14H), 1.23-1.05 (m, 4H), 0.99 (dd, J=14.4, 3.4 Hz, 1H), 0.93 (d, J=7.0 Hz, 6H), 0.90 (s, 3H), 0.65 (s, 3H).

¹³C NMR (100 MHz, CDCl₃) δ 173.8, 72.0, 68.4, 56.1, 50.5, 42.8, 41.7, 39.9, 39.8, 39.5, 39.3, 35.6, 35.5, 35.2, 34.8, 33.7, 32.9, 32.1, 31.8, 30.7, 28.4, 23.8, 22.9, 20.7, 20.2, 18.5, 13.9, 11.9.

HRMS (ESI): m/z calcd. C₂₈H₄₉NO₃Na (M+Na⁺) 470.3610, found 470.3614.

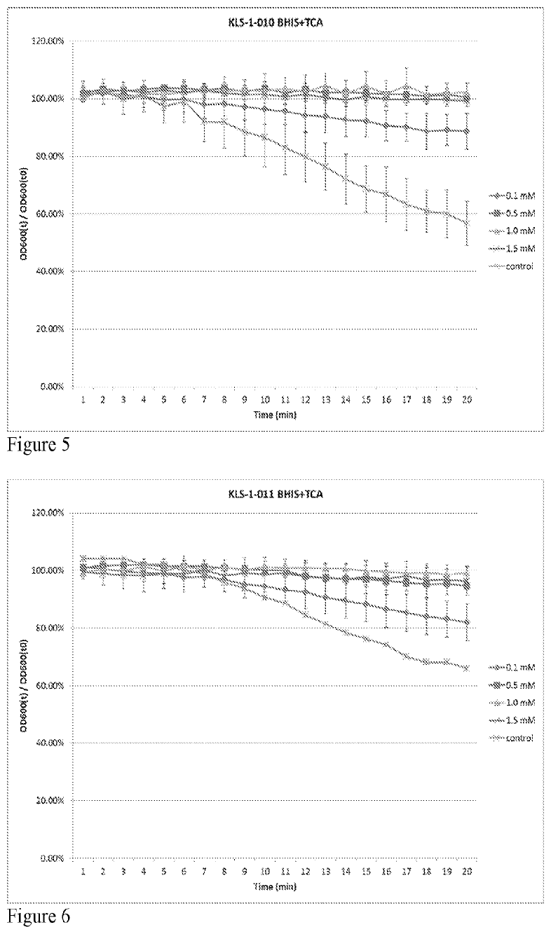

FIG. 5 is a graphical representation illustrating changes in optical density over time indicative of spore germination in the presence of an exemplary comparative compound as described in Comparative Example 3 at various concentrations. The graph illustrates essentially complete inhibition of spore germination by KLS-1-010 at 0.5 mM.

### Comparative Example 4: Compound Having the Structure

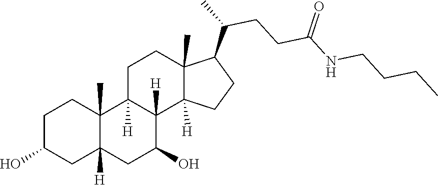

To a solution of UDCA (0.393 g, 1.00 mmol) in THF (10 mL) was added CDI (0.243 g, 1.50 mmol) and the reaction stirred for 1 hour at room temperature. To the solution was added butan-1-amine (0.198 mL, 2.00 mmol) and the reaction stirred for 18 hours at room temperature. The reaction was quenched with NH₄Cl (10 mL). The aqueous and organic layers were separated and the aqueous layer was extracted with EtOAc (3×10 mL). The combined organic layers were washed with NH₄Cl (3×5 mL), water (5 mL), then dried over MgSO₄, filtered, and concentrated. The crude material was purified by flash column chromatography on silica gel (2-5% MeOH/EtOAc over 35 minutes, then 10% MeOH/EtOAc flush) to obtain KLS-1-011 (0.162 g, 36% yield) as an off-white solid.

¹H NMR (400 MHz, CDCl₃) δ 5.45 (s, 1H), 3.59 (tq, J=10.0, 4.5, 4.1 Hz, 2H), 3.25 (td, J=7.1, 5.7 Hz, 2H), 2.23 (ddd, J=15.0, 10.4, 4.8 Hz, 1H), 2.11-1.97 (m, 2H), 1.91 (ddt, J=12.8, 9.4, 4.7 Hz, 1H), 1.79 (ddd, J=16.5, 7.0, 3.9 Hz, 5H), 1.74-1.55 (m, 4H), 1.55-1.20 (m, 16H), 1.20-0.98 (m, 3H), 0.97-0.88 (m, 9H), 0.68 (s, 3H).

¹³C NMR (100 MHz, CDCl₃) δ 173.8, 72.0, 68.4, 56.1, 50.5, 42.8, 41.7, 39.8, 39.5, 39.3, 35.6, 35.5, 35.2, 34.8, 33.7, 32.9, 32.1, 31.8, 30.7, 29.8, 28.4, 23.8, 22.9, 20.7, 20.2, 18.5, 13.9, 11.9.

HRMS (ESI): m/z calcd. C₂₈H₄₉NO₃Na (M+Na⁺) 470.3610, found 470.3615.

FIG. 6 is a graphical representation illustrating changes in optical density over time indicative of spore germination in the presence of an exemplary comparative compound as described in Comparative Example 4 at various concentrations. The graph illustrates essentially complete inhibition of spore germination by KLS-1-011 at 0.5 mM.

### Comparative Example 5: Mixture of Compounds Having the Structures

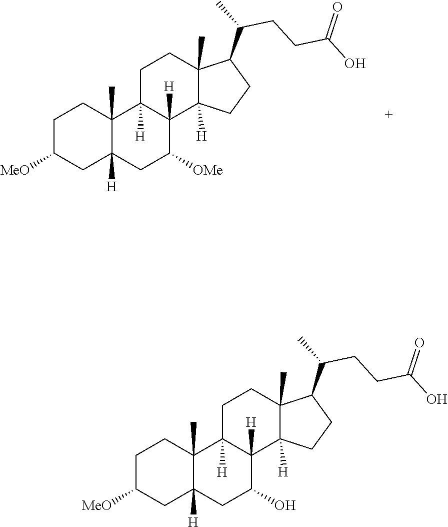

To a solution of KLS-1-008 (0.610 g, 1.50 mmol) in THF (7.5 mL) was added NaH (0.132 g, 3.30 mmol) and the reaction stirred at room temperature for 15 minutes. Methyl iodide (MeI, 0.197 mL, 3.15 mmol) was added dropwise, and the mixture stirred for 3 hours. The reaction was quenched by the addition of NH₄Cl (5 mL) and the aqueous layer was extracted with EtOAc (3×10 mL). The combined organic layers were washed with water (10 mL), dried over MgSO₄, filtered, and concentrated. The crude material was purified by flash column chromatography on silica gel (20-60% EtOAc in hexanes over 25 minutes, 80% EtOAc flush) to obtain a mixture of compounds. The mixture was repurified by flash column chromatography on silica gel (20-50% EtOAc in hexanes over 35 minutes, 80% EtOAc flush) to obtain KLS-1-012A (0.036 g, 5.7% yield) followed by the elution of KLS-1-012B (0.036 g, 5.9% yield). The compounds were separated by column chromatography and only pure KLS-1-012B was tested.

Data for KLS-1-012A:

¹H NMR (400 MHz, CDCl₃) δ 3.33 (s, 3H), 3.22 (s, 3H), 3.16 (q, J=2.7 Hz, 1H), 3.01 (tt, J=11.2, 4.2 Hz, 1H), 2.39 (ddd, J=15.4, 10.3, 5.0 Hz, 1H), 2.24 (ddd, J=15.8, 9.7, 6.4 Hz, 1H), 2.14 (td, J=13.1, 11.4 Hz, 1H), 1.98-1.65 (m, 8H), 1.61 (ddd, J=15.1, 5.5, 3.2 Hz, 1H), 1.57-1.36 (m, 6H), 1.33 (dt, J=14.3, 4.4 Hz, 4H), 1.23-1.10 (m, 4H), 1.09-0.99 (m, 1H), 0.92 (d, J=6.5 Hz, 4H), 0.90 (s, 3H), 0.63 (s, 3H). (Carboxylic acid —OH not observed).

¹³C NMR (100 MHz, CDCl₃) δ 180.0, 80.9, 56.1, 55.9, 55.5, 50.4, 42.7, 42.1, 39.8, 39.7, 35.6, 35.5, 35.4, 34.8, 33.8, 31.2, 31.0, 29.9, 28.3, 28.1, 26.9, 23.9, 23.2, 21.0, 18.5, 11.9.

HRMS (ESI): m/z calcd. C₂₆H₄₄NO₄Na (M+Na⁺) 443.3137, found 443.3116.

Data for KLS-1-012B:

¹H NMR (400 MHz, CDCl₃) δ 3.85 (q, J=3.0 Hz, 1H), 3.36 (s, 3H), 3.04 (tt, J=11.0, 4.3 Hz, 1H), 2.42 (ddd, J=15.5, 10.2, 5.1 Hz, 1H), 2.27 (ddd, J=15.9, 9.7, 6.4 Hz, 1H), 2.14 (td, J=13.3, 11.4 Hz, 1H), 2.05-1.70 (m, 9H), 1.65 (ddq, J=12.3, 7.0, 2.9 Hz, 1H), 1.54-1.43 (m, 3H), 1.43-1.22 (m, 8H), 1.16 (ddd, J=19.1, 10.7, 5.2 Hz, 3H), 0.95 (d, J=6.4 Hz, 3H), 0.92 (s, 3H), 0.67 (s, 3H). (Carboxylic acid —OH not observed).

¹³C NMR (100 MHz, CDCl₃) δ 178.5, 80.8, 68.7, 56.0, 55.7, 50.6, 42.9, 41.6, 39.9, 39.7, 36.0, 35.6, 35.6, 35.5, 34.9, 33.0, 31.0, 30.9, 28.4, 27.2, 23.9, 23.1, 20.8, 18.5, 12.0.

HRMS (ESI): m/z calcd. C₂₅H₄₂NO₄Na (M+Na⁺) 429.2981, found 429.2998.

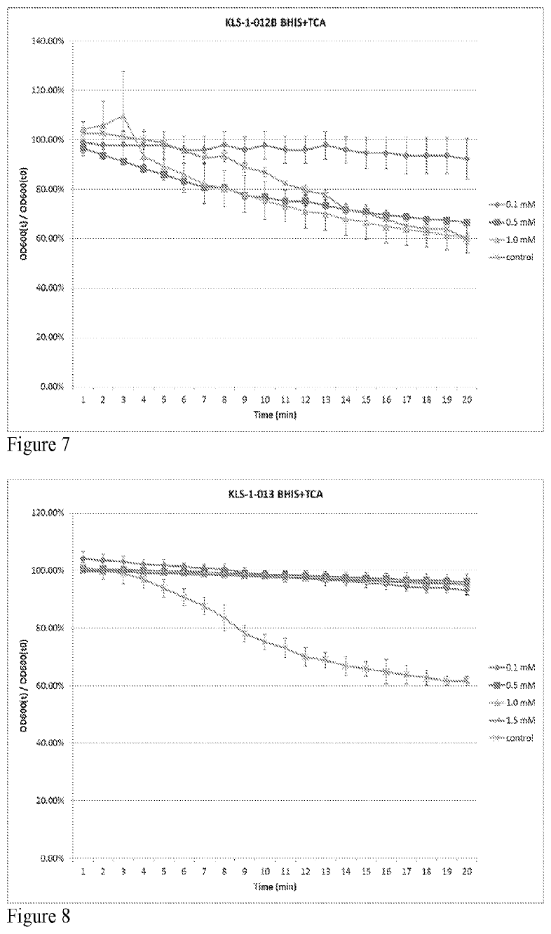

FIG. 7 is a graphical representation illustrating changes in optical density over time indicative of spore germination in the presence of exemplary comparative compound KLS-1-012B as described in Comparative Example 5 at various concentrations. The graph illustrates the partial inhibition of spore germination by KLS-1-012B at 0.1 mM.

### Example 3: Compound of Formula II Having the Structure

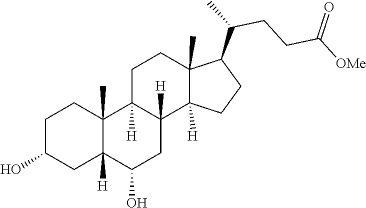

To a solution of HDCA (0.100 g, 0.255 mmol) in MeOH (26 mL) was added TsOH.H₂O (0.048 g, 0.255 mmol) and the reaction stirred at room temperature for 24 hours. The reaction was quenched by the addition of NaHCO₃ (5 mL) and the solvent was removed by rotary evaporation. The residue was partitioned between NaHCO₃ and EtOAc, and the aqueous layer was extracted with EtOAc (3×15 mL). The combined organic layers were washed with NaHCO₃ (3×10 mL), water (5 mL), dried over MgSO₄, filtered, and concentrated to an off-white foam. The crude material was purified by flash column chromatography on silica gel (20-60% EtOAc in hexanes over 25 minutes, 80% EtOAc in hexanes flush) to obtain KLS-1-013 (0.103 g, 99% yield) as a white solid.

¹H NMR (400 MHz, CDCl₃) δ 4.05 (dt, J=10.5, 4.6 Hz, 1H), 3.67 (s, 3H), 3.59 (dt, J=10.4, 4.5 Hz, 1H), 2.36 (ddd, J=15.4, 10.1, 5.0 Hz, 1H), 2.28-2.16 (m, 1H), 2.02-1.73 (m, 5H), 1.74-1.53 (m, 4H), 1.50-1.23 (m, 10H), 1.22-0.99 (m, 7H), 0.92 (app. d, J=7.0 Hz, 6H), 0.64 (s, 3H).

¹³C NMR (100 MHz, CDCl₃) δ 174.7, 71.5, 68.0, 56.2, 55.9, 51.5, 48.4, 42.8, 40.0, 39.8, 35.9, 35.6, 35.4, 34.9, 34.8, 31.1, 30.9, 30.2, 29.3, 28.1, 24.2, 23.5, 20.8, 18.2, 12.0.

HRMS (ESI): m/z calcd. C₂₅H₄₂NO₄Na (M+Na⁺) 429.2981, found 429.2984.

FIG. 8 is a graphical representation illustrating changes in optical density over time indicative of spore germination in the presence of the compound described in Example 3 at various concentrations. The graph illustrates essentially complete inhibition of spore germination by KLS-1-013 at 0.5 mM.

### Comparative Example 6: Mixture of Compounds Having the Structures

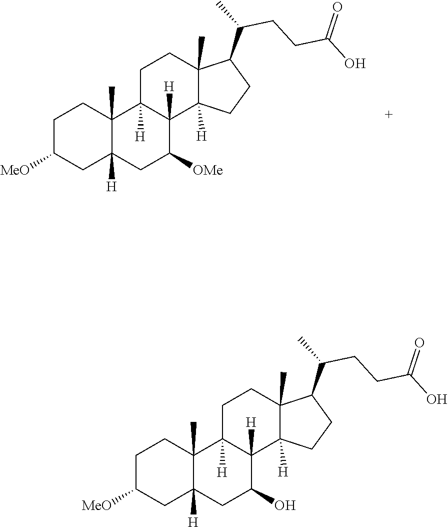

To a solution of KLS-1-009 (0.66 g, 1.623 mmol) in THF (8.1 mL) was added NaH (0.143 g, 3.57 mmol) and the reaction stirred at room temperature for 15 minutes. MeI (0.213 mL, 3.41 mmol) was added dropwise, and the mixture stirred for 3 hours. The reaction was quenched by the addition of NH₄Cl (5 mL) and the aqueous layer was extracted with EtOAc (3×10 mL). The combined organic layers were washed with water (10 mL), dried over MgSO₄, filtered, and concentrated. The crude material was purified by flash column chromatography on silica gel (25-67% EtOAc in hexanes) to obtain KLS-1-014A (0.018 g, 3% yield) followed by the elution of KLS-1-014B (0.224 g, 34% yield). The compounds were separated by column chromatography and pure samples of KLS-1-014A and KLS-1-014B were tested.

Data for KLS-1-014A:

¹H NMR (400 MHz, CDCl₃) δ 3.28 (s, 3H), 3.17 (s, 3H), 3.06 (qd, J=10.4, 9.7, 4.2 Hz, 1H), 2.92 (ddd, J=11.2, 9.0, 5.1 Hz, 1H), 2.33 (ddd, J=15.3, 10.3, 4.9 Hz, 1H), 2.18 (ddd, J=15.6, 9.5, 6.2 Hz, 1H), 1.90 (dt, J=12.8, 3.2 Hz, 1H), 1.83-1.64 (m, 6H), 1.63-1.52 (m, 3H), 1.47-1.24 (m, 9H), 1.13-0.89 (m, 6H), 0.86 (d, J=5.7 Hz, 6H), 0.59 (s, 3H).

¹³C NMR (100 MHz, CDCl₃) δ 179.7, 80.6, 80.3, 56.3, 55.8, 55.6, 55.4, 43.8, 42.4, 41.7, 40.4, 39.5, 35.5, 35.1, 34.6, 33.9, 32.3, 31.2, 31.1, 28.8, 26.8, 26.6, 23.6, 21.5, 18.6, 12.4.

HRMS (ESI): m/z calcd. C₂₆H₄₄NaO₄ (M+Na⁺) 443.3137, found 443.3111.

Data for KLS-1-014B: ¹H NMR (400 MHz, CDCl₃) δ 3.60 (ddd, J=11.5, 8.8, 5.0 Hz, 1H), 3.35 (s, 3H), 3.14 (ddt, J=15.2, 10.3, 4.9 Hz, 1H), 2.41 (ddd, J=15.3, 10.1, 5.0 Hz, 1H), 2.26 (ddd, J=15.8, 9.6, 6.4 Hz, 1H), 2.00 (dt, J=12.7, 3.2 Hz, 1H), 1.91 (ddd, J=13.2, 6.5, 3.5 Hz, 1H), 1.87-1.72 (m, 6H), 1.61 (ddd, J=15.6, 4.6, 2.2 Hz, 1H), 1.56-1.40 (m, 7H), 1.39-1.28 (m, 3H), 1.28-1.16 (m, 3H), 1.16-0.98 (m, 3H), 0.96 (s, 3H), 0.94 (d, J=6.1 Hz, 3H), 0.68 (s, 3H). (Carboxylic acid —OH peak not observed).

¹³C NMR (100 MHz, CDCl₃) δ 179.5, 80.3, 77.4, 71.6, 55.9, 55.8, 55.1, 44.0, 43.9, 42.6, 40.3, 39.3, 37.1, 35.5, 35.1, 34.6, 33.9, 31.2, 31.0, 28.8, 27.1, 26.8, 23.6, 21.4, 18.6, 12.3.

HRMS (ESI): m/z calcd. C₂₅H₄₂NO₄Na (M+Na⁺) 429.2981, found 429.2960.

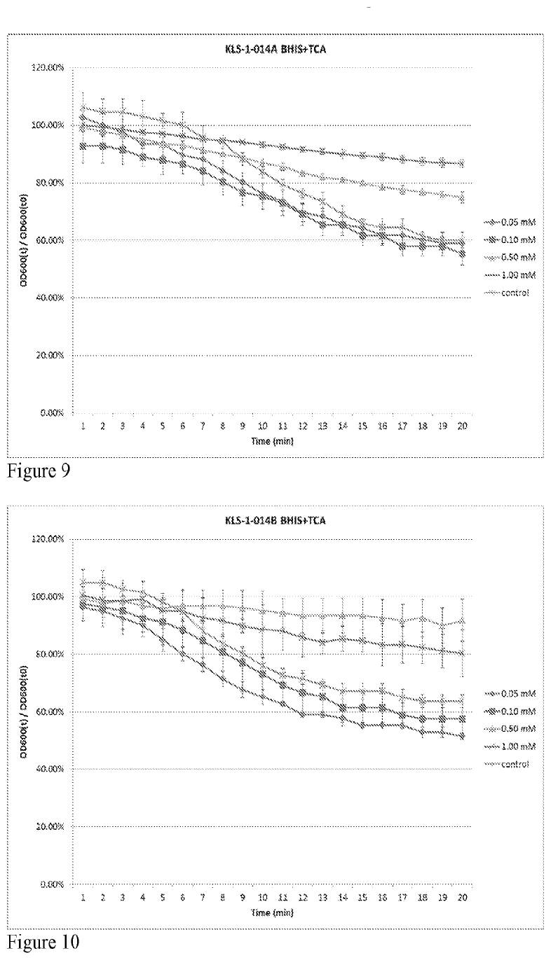

FIGS. 9-10 are graphical representations illustrating changes in optical density over time indicative of spore germination in the presence of exemplary comparative compounds as described in Comparative Example 6 at various concentrations. FIG. 9 illustrates the partial inhibition of spore germination by KLS-1-014A at 1.0 mM. FIG. 10 illustrates the partial inhibition of spore germination by KLS-1-014B at 0.50 mM.

### Example 4: Mixture of Compounds of Formula I Having the Structures

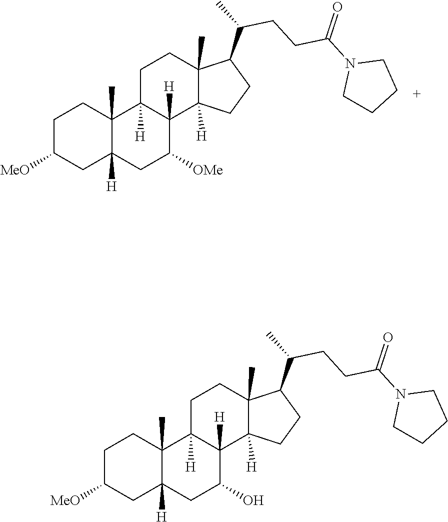

To a solution of KLS-1-015 (0.480 g, 1.08 mmol) in THF (10.7 mL) was added NaH (0.065 g, 1.62 mmol). The reaction stirred at room temperature for 15 minutes, and MeI (0.08 mL, 1.3 mmol) was added in one portion. The reaction stirred overnight 13 hours and quenched with NH₄Cl (8 mL). The organic and aqueous layers were separated and the aqueous layer was extracted with EtOAc (3×10 mL). The combined organic layers were washed with brine (10 mL), dried over MgSO₄, filtered, and concentrated. The crude material was purified by flash column chromatography on silica gel (1/3/0.1 DCM/EtOAc/MeOH over 35 minutes) to obtain KLS-1-017A (0.041 g, 8% yield) as white solid, followed by the elution of KLS-1-017B (0.327 g, 66% yield). The compounds were separated by column chromatography and pure samples of KLS-1-017A and KLS-1-017B were tested.

Data for KLS-1-017A: ¹H NMR (400 MHz, DMSO-D₆) δ 3.38 (t, J=6.8 Hz, 2H), 3.25 (t, J=6.9 Hz, 2H), 3.21 (s, 3H), 3.17 (d, J=5.2 Hz, 2H), 3.13 (s, 3H), 3.06 (tt, J=9.1, 4.5 Hz, 1H), 2.96 (ddd, J=14.4, 7.1, 3.1 Hz, 1H), 2.23 (ddd, J=15.5, 10.5, 5.1 Hz, 1H), 2.09 (ddd, J=15.6, 10.2, 5.9 Hz, 1H), 1.96-1.90 (m, 1H), 1.86 (p, J=6.6 Hz, 2H), 1.75 (q, J=6.8 Hz, 4H), 1.72-1.55 (m, 4H), 1.54-1.44 (m, 1H), 1.44-1.29 (m, 7H), 1.28-1.11 (m, 5H), 1.11-0.92 (m, 3H), 0.89 (d, J=6.7 Hz, 3H), 0.88 (s, 3H), 0.61 (s, 3H).

¹³C NMR (100 MHz, (DMSO-D₆) δ 170.7, 79.4, 79.3, 55.5, 54.9, 54.8, 54.7, 54.7, 48.6, 45.9, 45.2, 43.1, 41.5, 40.9, 35.0, 34.4, 33.9, 33.2, 31.7, 30.8, 30.6, 28.1, 26.4, 26.1, 25.7, 24.0, 23.2, 20.9, 18.5, 12.0.

Data for KLS-1-017B:

¹H NMR (400 MHz, CDCl₃) δ 3.59 (tt, J=9.0, 4.0 Hz, 1H), 3.44 (dt, J=16.1, 6.8 Hz, 4H), 3.35 (s, 3H), 3.12 (tt, J=10.5, 4.5 Hz, 1H), 2.31 (ddd, J=15.4, 10.8, 5.0 Hz, 1H), 2.16 (ddd, J=15.1, 10.3, 5.7 Hz, 1H), 2.03-1.89 (m, 4H), 1.89-1.73 (m, 8H), 1.61 (ddd, J=13.0, 5.2, 1.8 Hz, 1H), 1.55-1.30 (m, 10H), 1.30-0.97 (m, 6H), 0.95 (app. d, J=5.0 Hz, 6H), 0.68 (s, 3H).

¹H NMR (400 MHz, DMSO-D₆) δ 3.86 (d, J=6.8 Hz, 1H), 3.38 (t, J=6.7 Hz, 2H), 3.24 (app. t, J=6.9 Hz, 2H), 3.20 (s, 3H), 3.04 (td, J=10.4, 4.6 Hz, 1H), 2.23 (ddd, J=15.5, 10.5, 5.2 Hz, 1H), 2.16-2.03 (m, 1H), 1.97-1.55 (m, 12H), 1.51-0.95 (m, 16H), 0.89 (d, J=6.3 Hz, 6H), 0.62 (s, 3H).

¹³C NMR (100 MHz, CDCl₃) δ 172.4, 80.2, 71.6, 55.9, 55.8, 55.2, 46.8, 45.8, 44.0, 42.6, 40.3, 39.3, 37.1, 35.7, 35.1, 34.6, 34.6, 33.9, 31.9, 31.2, 28.8, 27.1, 26.9, 26.4, 24.6, 23.6, 21.4, 18.8, 12.4.

HRMS (ESI): m/z calcd. C₂₉H₄₉NO₃Na (M+Na⁺) 482.7048, found 482.3612.

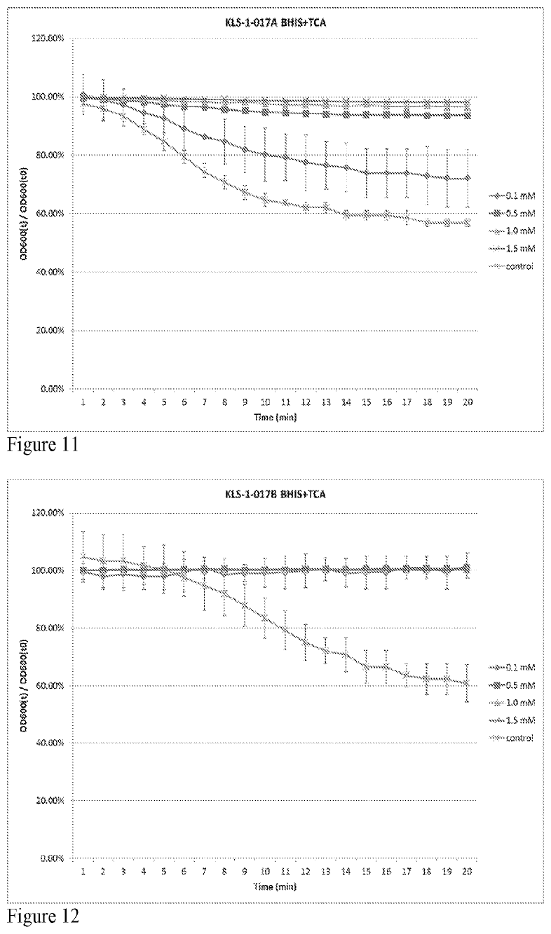

FIGS. 11-12 are graphical representations illustrating changes in optical density over time indicative of spore germination in the presence of the compounds described in Example 4 at various concentrations. FIG. 11 illustrates essentially complete inhibition of spore germination by KLS-1-017A at 0.5 mM. FIG. 12 illustrates essentially complete inhibition of spore germination by KLS-1-017B at 0.1 mM.

### Example 5: Mixture of Compounds of Formula I Having the Structures

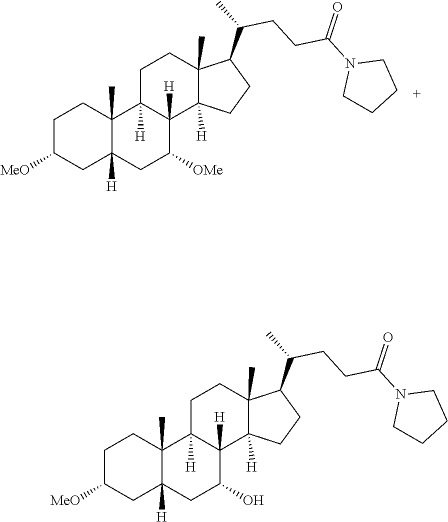

To a suspension of KLS-1-016 (0.300 g, 0.673 mmol) in THF (6.7 mL) at room temperature was added NaH (0.040 g, 1.01 mmol) in one portion. MeI (0.05 mL, 0.81 mmol) was added, and the reaction stirred for 19 hours. Additional NaH (0.040 g, 1.01 mmol) and MeI (0.05 mL, 0.81 mmol) were added, and the reaction stirred 9.5 hours. Another portion of NaH (0.040 g, 1.010 mmol) and MeI (0.05 mL, 0.81 mmol) were added. The reaction stirred 13 hours and was quenched by the addition of NH₄Cl (8 mL). The organic and aqueous layers were separated and the aqueous layer was extracted with EtOAc (3×10 mL). The combined organic layers were washed with brine (10 mL), dried over MgSO₄, filtered, and concentrated. The crude material was purified by flash column chromatography on silica gel (1/3/0.1 DCM/EtOAc/MeOH over 35 minutes) to obtain KLS-1-018A (0.168 g, 53% yield) as white solid, followed by the elution of KLS-1-018B (0.186 g, 60% yield).

Data for KLS-1-018A:

¹H NMR (400 MHz, CDCl₃) δ 3.45 (dt, J=16.5, 6.8 Hz, 4H), 3.34 (s, 3H), 3.24 (s, 3H), 3.18 (q, J=2.8 Hz, 1H), 3.01 (tt, J=11.1, 4.2 Hz, 1H), 2.31 (ddd, J=15.5, 10.9, 5.0 Hz, 1H), 2.24-2.08 (m, 2H), 2.01-1.67 (m, 12H), 1.63 (ddd, J=15.0, 5.5, 3.2 Hz, 1H), 1.59-1.41 (m, 5H), 1.42-1.15 (m, 8H), 1.05 (tt, J=10.3, 5.7 Hz, 1H), 0.94 (d, J=6.6 Hz, 3H), 0.91 (s, 3H), 0.65 (s, 3H).

¹³C NMR (100 MHz, CDCl₃) δ 172.5, 80.9 (2C), 56.1, 55.9, 55.6, 50.4, 46.8, 45.8, 42.7, 42.2, 39.8, 39.7, 35.7, 35.6, 35.5, 34.9, 33.9, 31.7, 31.1, 28.4, 28.1, 26.9, 26.4, 24.6, 23.9, 23.2, 21.1, 18.7, 11.9.

HRMS (ESI): m/z calcd. C₃₀H₅₁NNaO₃ (M+N⁺) 496.3767, found 496.3769.

Data for KLS-1-018B:

¹H NMR (400 MHz, CDCl₃) δ 2.31 (ddd, J=15.5, 10.8, 5.1 Hz, 1H), 2.23-2.06 (m, 2H), 2.05-1.90 (m, 5H), 1.90-1.69 (m, 7H), 1.69-1.57 (m, 1H), 1.54-1.42 (m, 4H), 1.43-1.26 (m, 6H), 1.24-1.07 (m, 5H), 0.95 (d, J=6.5 Hz, 3H), 0.92 (s, 3H), 0.67 (s, 3H).

¹³C NMR (100 MHz, CDCl₃) δ 172.4, 80.8, 68.7, 56.0, 55.7, 50.7, 46.8, 45.8, 42.9, 41.6, 39.9, 39.7, 36.1, 35.7, 35.6, 35.5, 34.9, 33.0, 31.7, 31.1, 28.4, 27.2, 26.4, 24.6, 24.0, 23.1, 20.8, 18.7, 12.0.

HRMS (ESI): m/z calcd. C₂₉H₄₉NO₃Na (M+Na⁺) 482.7048, found 482.3624.

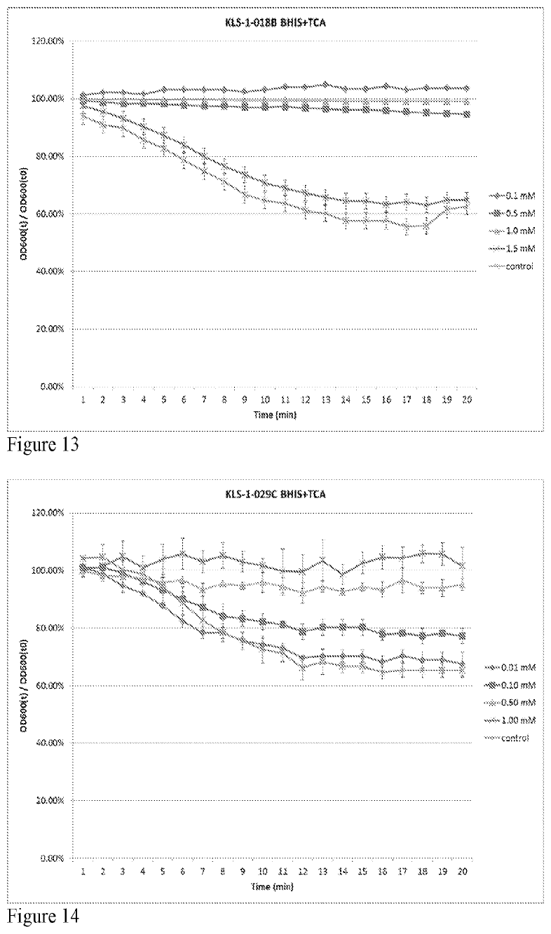

FIG. 13 is a graphical representation illustrating changes in optical density over time indicative of spore germination in the presence of the compound KLS-1-018B described in Example 5 at various concentrations. The graph illustrates essentially complete inhibition of spore germination by KLS-1-018B at 0.1 mM.

### Comparative Example 7: Mixture of Compounds Having the Structures

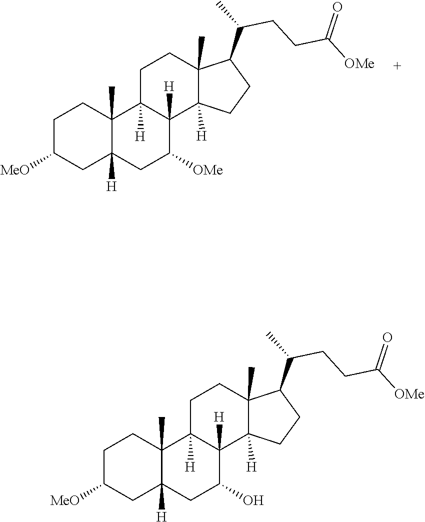

To a solution of KLS-1-008 (0.300 g, 0.738 mmol) and 2,6-di-tert-butylpyridine (0.33 mL, 1.48 mmol) in DCM (7.4 mL) was added methyl trifluoromethanesulfonate (0.10 mL, 0.89 mmol). The reaction stirred at room temperature for 24 hours and was quenched by the addition of 1M HCl (5 mL). The aqueous layer was extracted with DCM (3×10 mL) and the combined organic layers were washed with NaHCO₃ (2×10 mL), dried over MgSO₄, filtered, and concentrated. The crude material was purified by flash column chromatography on silica gel (0-50% EtOAc in DCM) to obtain KLS-1-029C (0.054 g, 17% yield) followed by the elution of KLS-1-029D (0.165 g, 53% yield). The compounds were separated by column chromatography and pure samples of KLS-1-029C and KLS-1-029D were tested.

Data for KLS-1-029C:

¹H NMR (400 MHz, CDCl₃) δ 3.66 (s, 3H), 3.33 (s, 3H), 3.23 (s, 3H), 3.17 (q, J=2.9 Hz, 1H), 3.01 (tt, J=11.2, 4.3 Hz, 1H), 2.35 (ddd, J=15.2, 10.2, 5.0 Hz, 1H), 2.29-2.08 (m, 2H), 1.97-1.66 (m, 8H), 1.62 (ddd, J=15.0, 5.4, 3.2 Hz, 1H), 1.57-1.39 (m, 5H), 1.37-1.10 (m, 8H), 1.08-0.99 (m, 1H), 0.92 (d, J=6.4 Hz, 3H), 0.90 (s, 3H), 0.63 (s, 3H).

¹³C NMR (100 MHz, CDCl₃) δ 175.0, 80.8 (2C), 68.7, 55.9, 55.7, 51.7, 50.6, 42.9, 41.6, 39.8, 39.7, 36.1, 35.6, 35.6, 35.5, 34.9, 33.0, 31.2, 31.2, 28.4, 27.2, 23.9, 23.1, 20.8, 18.5, 12.0.

HRMS (ESI): m/z calcd. C₂₇H₄₆NaO₄ (M+Na⁺) 457.3294, found 457.3290.

Data for KLS-1-029D:

¹H NMR (400 MHz, CDCl₃) δ 3.85 (dt, J=5.4, 2.5 Hz, 1H), 3.67 (s, 3H), 3.35 (s, 3H), 3.02 (tt, J=11.1, 4.3 Hz, 1H), 2.36 (ddd, J=15.3, 10.1, 5.1 Hz, 1H), 2.23 (ddd, J=15.7, 9.6, 6.6 Hz, 1H), 2.12 (td, J=13.2, 11.2 Hz, 1H), 2.04-1.89 (m, 3H), 1.89-1.72 (m, 5H), 1.64 (dddd, J=12.5, 9.9, 7.0, 3.0 Hz, 1H), 1.53-1.42 (m, 3H), 1.42-1.20 (m, 8H), 1.21-1.09 (m, 4H), 0.93 (d, J=6.6 Hz, 3H), 0.91 (s, 3H), 0.66 (s, 3H).

¹H NMR (400 MHz, DMSO-D₆) δ 4.10 (d, J=3.4 Hz, 1H), 3.62 (q, J=3.1 Hz, 1H), 3.57 (s, 3H), 3.20 (s, 3H), 2.92 (tt, J=11.1, 4.3 Hz, 1H), 2.32 (ddd, J=15.2, 9.6, 5.3 Hz, 1H), 2.26-2.09 (m, 2H), 1.94-1.86 (m, 1H), 1.86-1.57 (m, 9H), 1.46-1.31 (m, 5H), 1.29-1.15 (m, 4H), 1.15-1.03 (m, 3H), 0.99 (dq, J=11.8, 5.7 Hz, 1H), 0.88 (d, J=6.4 Hz, 3H), 0.85 (s, 3H), 0.60 (s, 3H).

¹³C NMR (100 MHz, CDCl₃) δ 175.0, 80.8, 68.7, 55.9, 55.7, 51.7, 50.6, 42.9, 41.6, 39.8, 39.7, 36.1, 35.6, 35.6, 35.5, 34.9, 33.0, 31.2, 31.2, 28.4, 27.2, 23.9, 23.1, 20.8, 18.5, 12.0.

HRMS (ESI): m/z calcd. C₂₆H₄₄NO₄Na (M+Na⁺) 443.3137, found 443.3121.

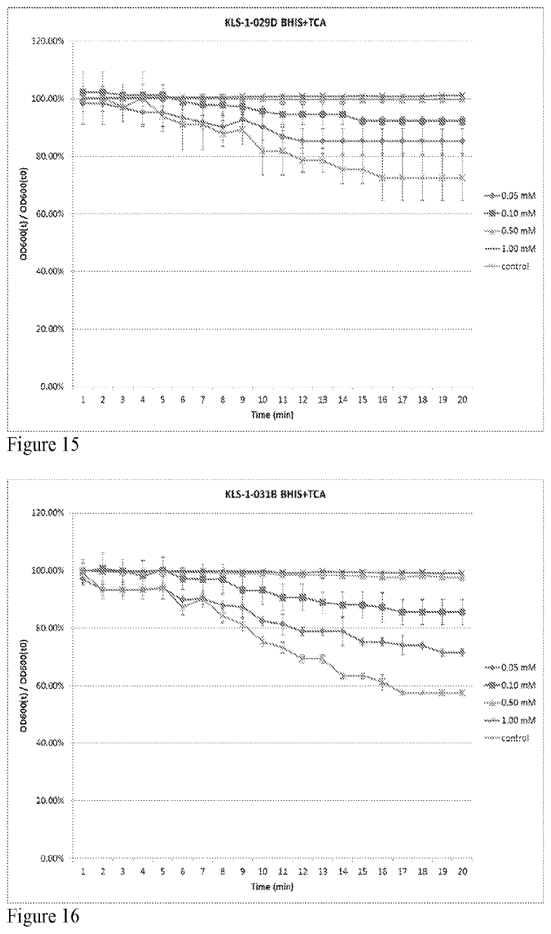

FIGS. 14-15 are graphical representations illustrating changes in optical density over time indicative of spore germination in the presence of exemplary comparative compounds as described in Comparative Example 7 at various concentrations. FIG. 14 illustrates essentially complete inhibition of spore germination by KLS-1-029C at 0.5 mM. FIG. 15 illustrates essentially complete inhibition of spore germination by KLS-1-029D at 0.50 mM.

### Comparative Example 8: Mixture of Compounds Having the Structures

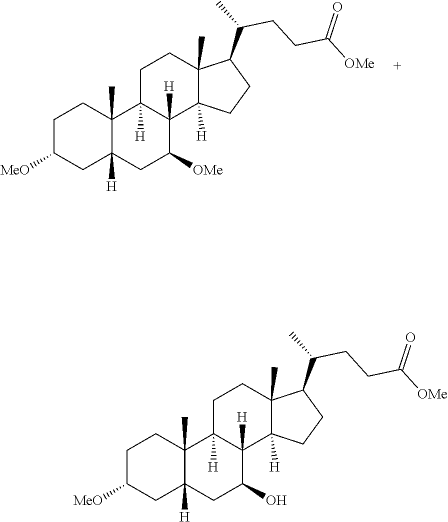

To a solution of KLS-1-009 (0.260 g, 0.639 mmol) and 2,6-di-tert-butylpyridine (0.29 mL, 1.28 mmol) in DCM (6.4 mL) was added methyl trifluoromethanesulfonate (0.07 mL, 0.67 mmol). The reaction stirred at room temperature for 24 hours and was quenched by the addition of 1M HCl (5 mL). The aqueous layer was extracted with DCM (3×10 mL) and the combined organic layers were washed with NaHCO₃ (2×10 mL), dried over MgSO₄, filtered, and concentrated. The crude material was purified by flash column chromatography on silica gel (0-50% EtOAc in DCM) to obtain KLS-1-031B (0.147 g, 53% yield) as a clear colorless oil, followed by the elution of KLS-1-031C (0.054 g, 21% yield) as clear, colorless oil that solidified over time to a white solid. The compounds were separated by column chromatography and pure samples of KLS-1-031B and KLS-1-031C were tested.

Data for KLS-1-031B:

¹H NMR (400 MHz, CDCl₃) δ 3.67 (s, 3H), 3.36 (s, 3H), 3.24 (s, 3H), 3.13 (tt, J=10.2, 4.4 Hz, 1H), 2.99 (ddd, J=11.3, 8.8, 5.0 Hz, 1H), 2.36 (ddd, J=15.2, 10.2, 4.9 Hz, 1H), 2.22 (ddd, J=15.6, 9.6, 6.4 Hz, 1H), 1.98 (dt, J=12.8, 3.3 Hz, 1H), 1.90-1.71 (m, 6H), 1.71-1.61 (m, 2H), 1.53-1.37 (m, 7H), 1.37-1.30 (m, 1H), 1.30-1.09 (m, 5H), 1.09-0.97 (m, 2H), 0.94 (s, 3H), 0.92 (d, J=6.4 Hz, 3H), 0.66 (s, 3H).

¹³C NMR (100 MHz, CDCl₃) δ 175.0, 80.6, 80.2, 56.4, 55.8, 55.7, 55.4, 51.7, 43.8, 42.4, 41.7, 40.5, 39.5, 35.6, 35.1, 34.6, 34.0, 32.3, 31.3, 31.3, 28.8, 26.9, 26.6, 23.6, 21.5, 18.6, 12.4.

HRMS (ESI): m/z calcd. C₂₇H₄₆NaO₄ (M+Na⁺) 457.3294, found 457.3278.

Data for KLS-1-031C:

¹H NMR (400 MHz, CDCl₃) δ 3.60 (s, 3H), 3.52 (s, 1H), 3.27 (s, 3H), 3.05 (tt, J=10.6, 4.6 Hz, 1H), 2.29 (ddd, J=15.2, 10.1, 5.0 Hz, 1H), 2.15 (ddd, J=15.6, 9.5, 6.5 Hz, 1H), 1.92 (dt, J=12.6, 3.3 Hz, 1H), 1.83 (ddt, J=12.7, 9.3, 4.6 Hz, 1H), 1.78-1.64 (m, 6H), 1.57-1.44 (m, 2H), 1.44-1.32 (m, 7H), 1.32-1.20 (m, 3H), 1.17-1.03 (m, 2H), 1.04-0.90 (m, 3H), 0.88 (s, 3H), 0.85 (d, J=6.4 Hz, 3H), 0.60 (s, 3H).

¹H NMR (400 MHz, DMSO-D₆) δ 3.87 (d, J=6.9 Hz, 1H), 3.57 (s, 3H), 3.31-3.22 (m, 1H), 3.20 (s, 3H), 3.05 (tt, J=9.7, 5.3 Hz, 1H), 2.32 (ddd, J=15.2, 9.7, 5.2 Hz, 1H), 2.20 (ddd, J=15.7, 9.3, 6.8 Hz, 1H), 1.91 (dd, J=11.4, 3.6 Hz, 1H), 1.88-1.79 (m, 1H), 1.78-1.59 (m, 6H), 1.47-1.28 (m, 7H), 1.27-1.09 (m, 6H), 1.06-0.88 (m, 3H), 0.87 (app. d, J=6.8 Hz, 6H), 0.61 (s, 3H).

¹³C NMR (100 MHz, CDCl₃) δ 174.9, 80.2, 71.6, 55.9, 55.8, 55.1, 51.7, 44.0, 44.0, 42.6, 40.3, 39.3, 37.2, 35.5, 35.1, 34.6, 33.9, 31.3, 31.2, 28.8, 27.1, 26.9, 23.6, 21.4, 18.6, 12.3.

HRMS (ESI): m/z calcd. C₂₆H₄₄NO₄Na (M+Na⁺) 443.3137, found 443.3146.

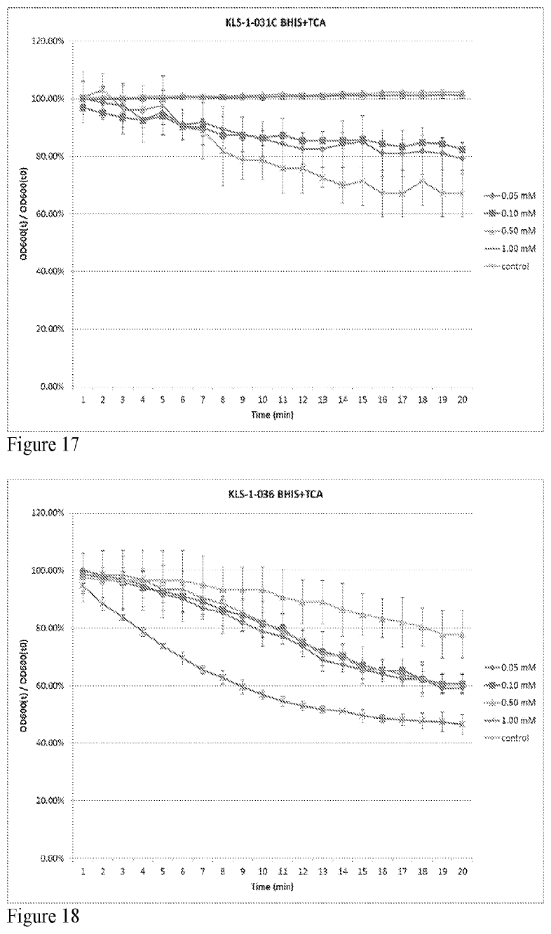

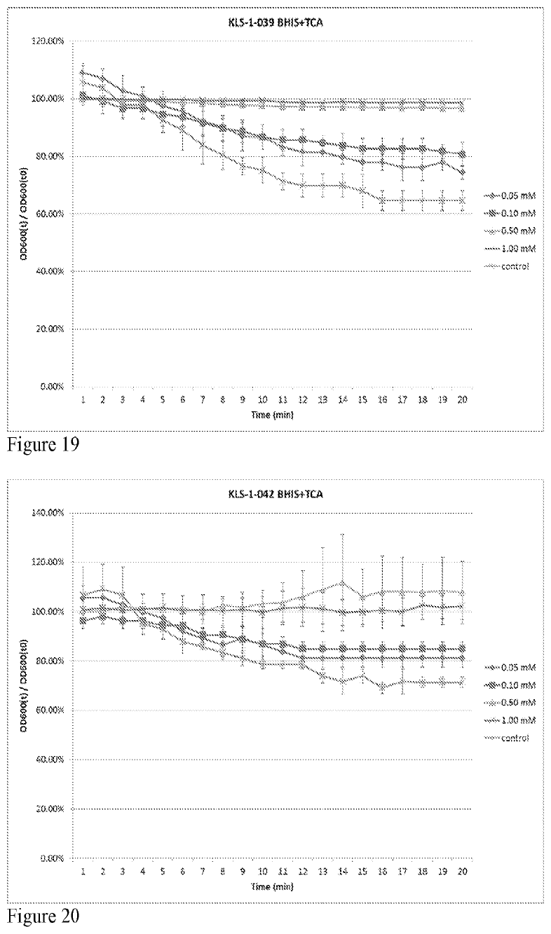

FIGS. 16-17 are graphical representations illustrating changes in optical density over time indicative of spore germination in the presence of exemplary comparative compounds as described in Comparative Example 8 at various concentrations. FIG. 16 illustrates essentially complete inhibition of spore germination by KLS-1-031B at 0.5 mM. FIG. 17 illustrates essentially complete inhibition of spore germination by KLS-1-031C at 0.5 mM.

### Comparative Example 9: Compound Having the Structure

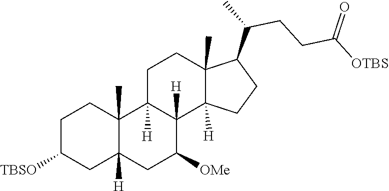

To a solution of tert-butyldimethylsilyl (R)-4-((3R,5R,7S,8R,9S,10S,13R,14S,17R)-3-((tert-butyldimethylsilyl)oxy)-7-hydroxy-10,13-dimethylhexadecahydro-1H-cyclopenta[a]phenanthren-17-yl)pentanoate (0.300 g, 0.483 mmol) and 2,6-di-tert-butylpyridine (0.22 mL, 0.97 mmol) in DCM (5 mL) was methyl trifluoromethanesulfonate (0.06 mL, 0.51 mmol). The reaction stirred at room temperature for 24 hours and was quenched by the addition of 1M HCl (5 mL). The aqueous layer was extracted with DCM (3×10 mL) and the combined organic layers were washed with NaHCO₃ (2×10 mL), dried over MgSO₄, filtered, and concentrated. The crude material was purified by flash column chromatography on silica gel (0-50% EtOAc in DCM) to obtain KLS-1-032B (0.207 g, 68% yield) as a white solid.

¹H NMR (400 MHz, CDCl₃) δ 3.54 (tt, J=10.2, 4.7 Hz, 1H), 3.24 (s, 3H), 3.01 (ddd, J=11.0, 9.1, 5.0 Hz, 1H), 2.36 (ddd, J=15.2, 9.9, 5.1 Hz, 1H), 2.22 (ddd, J=15.6, 9.4, 6.6 Hz, 1H), 1.97 (dt, J=12.5, 3.1 Hz, 1H), 1.90-0.97 (m, 26H), 0.94 (s, 9H), 0.92 (s, 3H), 0.90 (s, 9H), 0.66 (s, 3H), 0.27 (d, J=1.2 Hz, 6H), 0.07 (d, J=1.5 Hz, 6H).

¹³C NMR (100 MHz, CDCl₃) δ 174.8, 80.3, 72.5, 56.1, 55.4, 55.3, 43.6, 42.4, 41.5, 40.2, 39.2, 37.8, 35.3, 35.1, 34.1, 33.1, 32.1, 31.2, 30.9, 28.6, 26.4, 26.0, 26.0, 25.6, 25.6, 23.4, 23.4, 21.3, 18.4, 18.3, 17.6, 12.2, -4.6, -4.6, -4.8, -4.8.

### Comparative Example 10: Compound Having the Structure

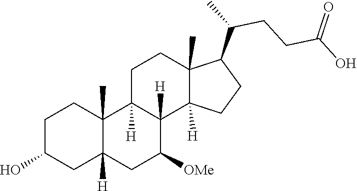

To a solution of KLS-1-032B (0.174 g, 0.274 mmol) in THF (2.7 mL) was added tetra-n-butylammoinum fluoride (TBAF, approximately 1 M in THF, 0.58 mL, 0.58 mmol) and the reaction mixture stirred for 3 hours. Additional TBAF (approximately 1 M in THF, 0.58 mL, 0.58 mmol) was added, and the reaction mixture stirred at room temperature for 20 hours. The solvent was evaporated and the crude material was purified by flash column chromatography on silica gel (0-10% MeOH in DCM) to obtain KLS-1-036B (0.094 g, 84% yield).

¹H NMR (400 MHz, CDCl₃) δ 3.61 (tt, J=10.4, 4.8 Hz, 1H), 3.25 (s, 3H), 3.01 (ddt, J=13.0, 9.2, 5.0 Hz, 2H), 2.40 (ddd, J=15.1, 10.0, 4.9 Hz, 1H), 2.27 (ddd, J=15.7, 9.5, 6.3 Hz, 1H), 1.99 (dt, J=12.5, 3.1 Hz, 1H), 1.90-1.75 (m, 4H), 1.74-1.62 (m, 5H), 1.62-1.51 (m, 3H), 1.51-1.35 (m, 7H), 1.22-1.08 (m, 3H), 1.08-1.00 (m, 2H), 0.96-0.91 (m, 6H), 0.67 (s, 3H).

¹H NMR (400 MHz, DMSO-D₆) δ 11.94 (s, 1H), 4.45 (d, J=4.5 Hz, 1H), 3.22 (d, J=79.8 Hz, 6H), 2.94 (td, J=10.3, 5.0 Hz, 2H), 2.23 (ddd, J=15.2, 9.7, 5.3 Hz, 1H), 2.15-2.04 (m, 1H), 1.93 (dd, J=11.6, 3.7 Hz, 1H), 1.82-1.64 (m, 3H), 1.64-1.52 (m, 2H), 1.52-1.45 (m, 2H), 1.36 (dddd, J=26.7, 20.5, 12.5, 8.3 Hz, 8H), 1.26-1.09 (m, 5H), 0.88 (d, J=6.7 Hz, 3H), 0.86 (s, 3H), 0.61 (s, 3H).

¹³C NMR (100 MHz, CDCl₃) δ 178.7, 80.6, 71.8, 56.4, 55.7, 55.4, 43.9, 42.4, 41.7, 40.5, 39.6, 37.5, 35.5, 35.1, 34.3, 32.2, 31.1, 30.6, 26.6, 25.5, 23.6, 20.3, 18.6, 13.8, 12.4.

HRMS (ESI): m/z calcd. C₂₅H₄₂NO₄Na (M+Na⁺) 429.2981, found 429.2965.

FIG. 18 is a graphical representation illustrating changes in optical density over time indicative of spore germination in the presence of an exemplary comparative compound as described in Comparative Example 10 at various concentrations. The graph illustrates the partial inhibition of spore germination by KLS-1-036 at 0.5 mM.

### Comparative Example 11: Compound Having the Structure

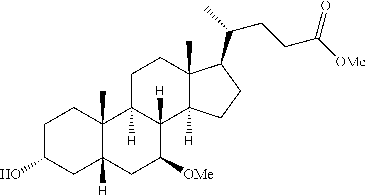

To a solution of KLS-1-036 (50 mg, 0.12 mmol) in MeOH (2.6 mL) was added pTsOH (0.050 g, 0.263 mmol) and the reaction stirred at room temperature for 24 hours. The solvent was removed by rotary evaporation. The crude material was purified by flash column chromatography on silica gel (33% EtOAc in DCM as eluent) to obtain KLS-1-039 (0.031 g, 60% yield) as a white solid.

¹H NMR (400 MHz, CDCl₃) δ 3.67 (s, 3H), 3.59 (tt, J=10.5, 4.7 Hz, 1H), 3.24 (s, 3H), 2.99 (ddd, J=11.3, 9.0, 5.2 Hz, 1H), 2.36 (ddd, J=15.2, 10.3, 4.9 Hz, 1H), 2.22 (ddd, J=15.5, 9.6, 6.4 Hz, 1H), 1.98 (dt, J=12.5, 3.3 Hz, 1H), 1.90-0.97 (m, 24H), 0.96-0.87 (m, 6H), 0.66 (s, 3H).

¹³C NMR (100 MHz, CDCl₃) δ 175.0, 80.5, 71.7, 56.4, 55.6, 55.4, 51.7, 43.8, 42.4, 41.7, 40.5, 39.6, 37.5, 35.5, 35.1, 34.3, 32.2, 31.3, 31.3, 30.6, 28.7, 26.6, 23.5, 21.5, 18.6, 12.4.

HRMS (ESI): m/z calcd. C₂₆H₄₄NaO₄ (M+Na⁺) 443.3137, found 443.3119.

FIG. 18 is a graphical representation illustrating changes in optical density over time indicative of spore germination in the presence of an exemplary comparative compound as described in Comparative Example 11 at various concentrations. The graph illustrates the complete inhibition of spore germination by KLS-1-039 at 0.5 mM.

### Comparative Example 12: Compound Having the Structure

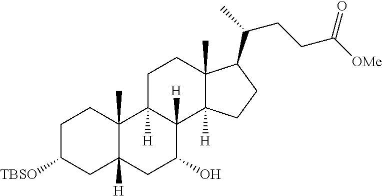

To a solution of KLS-1-008 (0.495 g, 1.217 mmol) in N,N-dimethylformamide (DMF, 1 mL) was added imidazole (0.398 g, 5.84 mmol) and tert-butyldimethylsilyl chloride (TBSCl, 0.229 g, 1.522 mmol). The reaction mixture stirred at room temperature for 3 hours and was poured into a separatory funnel containing ice water. The aqueous layer was extracted with EtOAc (3×10 mL), and the combined organic layers were washed with water (5×5 mL), dried over MgSO₄, filtered, and concentrated. The crude material was purified by flash column chromatography on silica gel (0-25% EtOAc in DCM) to obtain KLS-1-033 (0.471 g, 74% yield) as a white solid.

¹H NMR (400 MHz, CDCl₃) δ 3.84 (p, J=3.0 Hz, 1H), 3.67 (s, 3H), 3.44 (tt, J=10.9, 4.5 Hz, 1H), 2.36 (ddd, J=15.2, 10.1, 5.1 Hz, 1H), 2.29-2.12 (m, 2H), 2.01-1.92 (m, 3H), 1.92-1.75 (m, 4H), 1.70-1.53 (m, 3H), 1.53-1.43 (m, 4H), 1.43-1.29 (m, 5H), 1.29-1.23 (m, 1H), 1.20-1.10 (m, 4H), 0.93 (d, J=6.4 Hz, 3H), 0.89 (s, 3H), 0.89 (s, 9H), 0.66 (s, 3H), 0.05 (s, 6H).

### Comparative Example 13: Compound Having the Structure

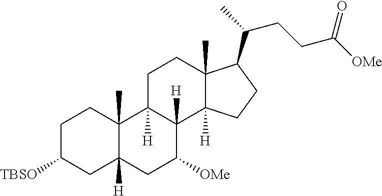

To a solution of KLS-1-033 (0.471 g, 0.904 mmol) and 2,6-di-tert-butylpyridine (0.41 mL, 1.81 mmol) in DCM (9 mL) was added methyl trifluoromethanesulfonate (0.11 mL, 0.95 mmol). The reaction mixture stirred overnight at room temperature. Additional 2,6-di-tert-butylpyridine (0.41 mL, 1.81 mmol) and methyl trifluoromethanesulfonate (0.11 mL, 0.95 mmol) were added, and the reaction stirred for 5 hours. Triethylamine (0.25 mL, 1.81 mmol) was added, and the reaction stirred overnight at room temperature for 7 days. The reaction mixture was quenched by the addition of water and stirred 15 minutes at room temperature. The aqueous layer was extracted with DCM (3×15 mL) and the combined organic layers were dried over MgSO₄, filtered, and concentrated. The crude material was purified by flash column chromatography on silica gel (0-20% EtOAc in DCM) to obtain KLS-1-035 (0.057 g, 12% yield), followed by the elution of the starting material (0.235, 50% recovery).

¹H NMR (400 MHz, CDCl₃) δ 3.66 (s, 3H), 3.42 (tt, J=10.8, 4.5 Hz, 1H), 3.24 (s, 3H), 3.17 (q, J=2.9 Hz, 1H), 2.35 (ddd, J=15.2, 10.2, 5.0 Hz, 1H), 2.28-2.13 (m, 2H), 1.94-1.66 (m, 4H), 1.66-0.99 (m, 16H), 0.91 (d, J=6.3 Hz, 3H), 0.88 (s, 6H), 0.88 (s, 9H), 0.63 (s, 3H), 0.04 (d, J=1.7 Hz, 6H).

¹³C NMR (100 MHz, CDCl₃) δ 175.0, 77.6, 73.1, 56.0, 55.9, 51.7, 50.4, 42.7, 42.3, 39.8, 39.6, 38.9, 35.7, 35.6, 35.2, 33.9, 31.3, 31.2, 31.2, 28.4, 28.1, 26.2, 26.2, 26.2, 23.9, 23.1, 21.0, 18.5, 18.4, 11.9, -4.2, -4.2.

### Comparative Example 14: Compound Having the Structure

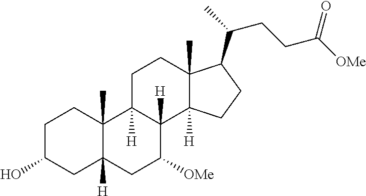

To a solution of KLS-1-035 (0.052 g, 0.097 mmol) in THF (1 mL) was added TBAF (approximately 1 M in THF, 0.11 mL, 0.11 mmol). The reaction stirred at room temperature for 24 hours and was concentrated under reduced pressure. The crude material was purified by flash column chromatography on silica gel (33% EtOAc in DCM) to obtain KLS-1-042 (0.030 g, 73% yield) as a clear, colorless oil.

¹H NMR (400 MHz, CDCl₃) δ 3.67 (s, 3H), 3.44 (tt, J=10.8, 4.6 Hz, 1H), 3.26 (s, 3H), 3.19 (d, J=2.9 Hz, 1H), 2.35 (ddd, J=15.3, 10.2, 5.0 Hz, 1H), 2.28-2.07 (m, 2H), 2.00-0.97 (m, 24H), 0.92 (d, J=7.4 Hz, 6H), 0.64 (s, 3H).

¹H NMR (400 MHz, DMSO-D₆) δ 4.34 (d, J=4.8 Hz, 1H), 3.57 (s, 3H), 3.17 (s, 5H), 2.33 (ddd, J=15.2, 9.6, 5.2 Hz, 1H), 2.20 (ddd, J=15.8, 9.3, 6.9 Hz, 1H), 1.99 (q, J=12.7 Hz, 1H), 1.93-0.96 (m, 20H), 0.87 (d, J=6.6 Hz, 6H), 0.85 (s, 3H), 0.60 (s, 3H).

¹³C NMR (100 MHz, CDCl₃) δ 175.0, 77.7, 72.3, 56.1, 55.9, 51.7, 50.5, 42.7, 42.2, 39.8, 39.6, 38.7, 35.6, 35.5, 35.2, 34.0, 31.2 (2C), 31.1, 28.3, 28.0, 23.9, 23.1, 21.1, 18.5, 11.9.

HRMS (ESI): m/z calcd. C₂₆H₄₄NaO₄ (M+Na⁺) 443.3137, found 443.3118.

FIG. 18 is a graphical representation illustrating changes in optical density over time indicative of spore germination in the presence of an exemplary comparative compound as described in Comparative Example 14 at various concentrations. The graph illustrates essentially complete inhibition of spore germination by KLS-1-042 at 0.5 mM.

### Comparative Example 15: Compound Having the Structure

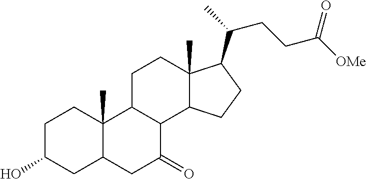 [SMILES: COC(=O)CC[C@@H](C)[C@H]1CCC2C3C(=O)CC4C[C@H](O)CC[C@]4(C)C3CC[C@@]21C]

3-alpha-7-oxo CDCA (0.800 g, 2.05 mmol) was dissolved in MeOH (20 mL) and pTsOH (0.051 g, 0.266 mmol) was added. The reaction mixture stirred at room temperature for 12 hours and the solvent was removed under reduced pressure. The crude material was purified by flash column chromatography on silica gel (0-40% EtOAc in DCM, 26 minutes) to obtain KLS-2-016 (0.671 g, 81% yield) as a white foam.

¹H NMR (400 MHz, CDCl₃) δ 3.67 (s, 3H), 3.61 (dq, J=10.7, 5.1 Hz, 1H), 2.86 (dd, J=12.5, 6.0 Hz, 1H), 2.37 (qd, J=10.7, 9.9, 6.2 Hz, 2H), 2.29-2.15 (m, 2H), 2.08-1.05 (m, 24H), 1.02-0.83 (m, 4H), 0.66 (s, 3H).

¹³C NMR (100 MHz, CDCl₃) δ 212.1, 174.9, 71.2, 55.0, 51.7, 49.7, 49.1, 46.3, 45.6, 42.9, 42.9, 39.2, 37.6, 35.4, 35.4, 34.4, 31.3, 31.2, 30.1, 28.5, 25.0, 23.3, 21.9, 18.6, 12.3.

### Example 6: Mixture of Compounds of Formula III Having the Structures

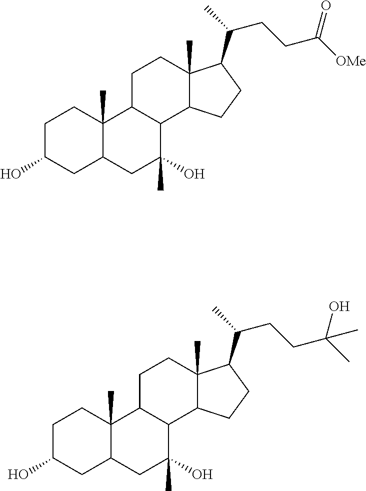 [SMILES: COC(=O)CC[C@@H](C)[C@H]1CCC2C3C(CC[C@@]21C)[C@@]1(C)CC[C@@H](O)CC1C[C@@]3(C)O ; C[C@H](CCC(C)(C)O)[C@H]1CCC2C3C(CC[C@@]21C)[C@@]1(C)CC[C@@H](O)CC1C[C@@]3(C)O]

To a solution of KLS-2-016 (0.671 g, 1.66 mmol) in diethyl ether (Et₂O, 17 mL) at room temperature was added methylmagnesium bromide (MeMgBr, 3.0 M in THF, 1.7 mL, 5.0 mmol) dropwise. The reaction was stirred for 2 hours and was quenched by the addition of 1M HCl (10 mL). The aqueous layer was extracted with Et₂O (3×10 mL) and the combined organic layers were dried over MgSO₄, filtered, and concentrated. The crude material was purified by flash column chromatography on silica gel (0-100% EtOAc in DCM) to obtain recovered starting material (160 mg, 24% recovery), followed by the elution of KLS-2-018A (0.107 g, 15% yield) and KLS-2-018B (0.130 g, 19% yield) as white foams.

Data for KLS-2-018A:

¹H NMR (400 MHz, DMSO-D₆) δ 4.31 (d, J=4.7 Hz, 1H), 3.57 (s, 3H), 3.21 (dq, J=11.1, 5.3 Hz, 1H), 2.32 (ddd, J=15.2, 9.7, 5.2 Hz, 1H), 2.27-2.09 (m, 2H), 2.02-1.61 (m, 6H), 1.52-0.96 (m, 20H), 0.89 (d, J=6.3 Hz, 4H), 0.80 (s, 3H), 0.62 (s, 3H).

¹³C NMR (100 MHz, DMSO-D₆) δ 173.7, 70.9, 70.9, 70.2, 54.4, 51.2, 50.8, 44.2, 43.3, 43.1, 41.9, 38.2, 35.6, 35.3, 34.8, 34.3, 33.1, 30.6, 30.4, 30.4, 28.0, 27.3, 22.8, 20.8, 18.3, 12.0.

HRMS (ESI): m/z calcd. C₂₆H₄₄NO₄Na (M+Na⁺) 443.3137, found 443.3116.

Data for KLS-2-018B:

¹H NMR (400 MHz, DMSO-D₆) δ 4.31 (d, J=4.7 Hz, 1H), 4.01 (s, 1H), 3.65 (s, 1H), 3.32 (s, 3H), 3.20 (tt, J=9.5, 4.7 Hz, 1H), 2.18 (q, J=12.7 Hz, 1H), 2.01-1.60 (m, 6H), 1.51-1.13 (m, 18H), 1.04 (s, 3H), 1.04 (s, 3H), 0.89 (d, J=6.5 Hz, 3H), 0.80 (s, 3H), 0.62 (s, 3H).

¹³C NMR (100 MHz, DMSO-D₆) δ 70.9, 70.2, 68.8, 64.9, 54.5, 50.8, 44.2, 43.2, 43.1, 41.9, 38.2, 35.6, 35.5, 35.3, 34.3, 33.2, 30.4, 29.8, 29.5, 29.1, 28.1, 27.3, 22.8, 20.8, 18.9, 15.2, 12.1.

HRMS (ESI): m/z calcd. C₂₇H₄₈NaO₃ (M+Na⁺) 443.3501, found 443.3483.

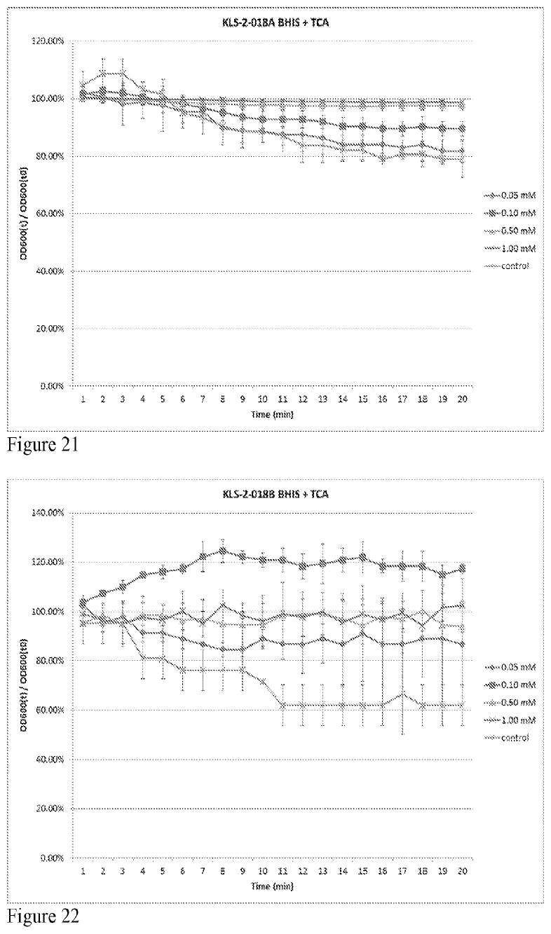

FIGS. 21 and 22 are graphical representations illustrating changes in optical density over time indicative of spore germination in the presence of the compound described in Example 6 at various concentrations. FIG. 21 illustrates essentially complete inhibition of spore germination by KLS-2-018A at 0.5 mM.

FIG. 22 illustrates essentially complete inhibition of spore germination by KLS-2-018B at 0.1 mM.

### Example 7: Compound of Formula I Having the Structure

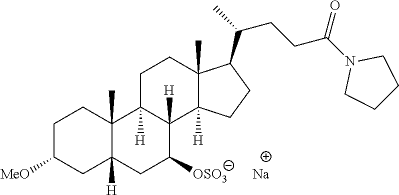

To a solution of KLS-1-017B (0.050 g, 0.109 mmol) in pyridine (0.544 mL) was added dropwise chlorosulfonic acid (0.05 mL, 0.71 mmol). The reaction mixture was heated to 50° C. for 30 minutes, then cooled to room temperature. The reaction was terminated by the addition of water (1 mL) and the solvents were removed by rotary evaporation. The crude material was purified by flash column chromatography on C₁₈ silica gel (5-100% (20 mM triethylammonium acetate buffer in acetonitrile) in filtered deionized water as eluent) and the solvents were lyophilized to obtain KLS-2-001 (0.054 g, 88% yield) as an off-white solid.

¹H NMR (400 MHz, D₂O) δ 4.32 (app. q, J=9.9, 8.5 Hz, 1H), 3.51 (t, J=6.9 Hz, 2H), 3.40 (d, J=7.0 Hz, 2H), 3.36 (s, 3H), 3.31 (s, 1H), 2.37 (d, J=10.4 Hz, 1H), 2.32-2.16 (m, 1H), 2.14-1.05 (m, 28H), 1.00 (app. d, J=6.6 Hz, 6H), 0.72 (s, 3H).

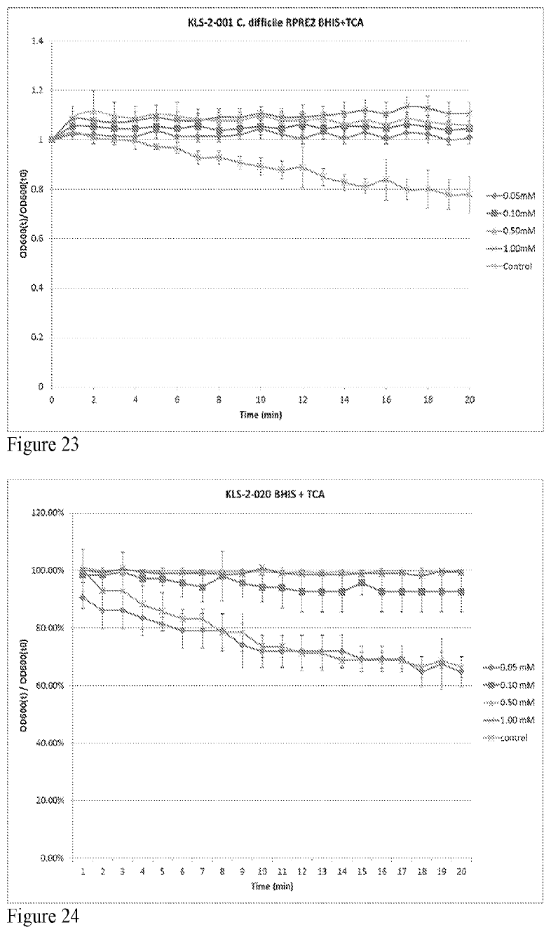

FIG. 23 is a graphical representation illustrating changes in optical density over time indicative of spore germination in the presence of the compound described in Example 7 at various concentrations. The graph illustrates essentially complete inhibition of spore germination by KLS-2-001 at 0.05 mM.

### Comparative Example 16: Compound Having the Structure

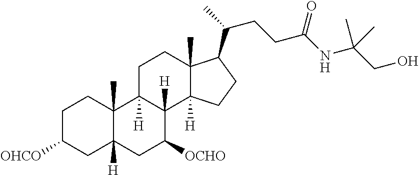

UDCA (1.00 g, 2.55 mmol) was dissolved in formic acid (20 mL) and perchloric acid (0.05 mL, 0.76 mmol) was added. The solution was heated to 50° C. for 12 hours, then cooled to 40° C. Acetic anhydride (10 mL) was added over 10 minutes and the solution was cooled to room temperature. The reaction was quenched with water (100 mL) and the aqueous layer was extracted with Et₂O (3×100 mL). The combined organic layers were washed with water (10×50 mL), dried over Na₂SO₄, filtered, and concentrated. The crude material was used for the next step without further purification. Thionyl chloride (1.71 mL, 23.4 mmol) was added dropwise to a solution of the crude material in benzene (12 mL) and the reaction was heated to reflux for 4 hours. The reaction was cooled to room temperature and the solvent was evaporated. The residual thionyl chloride was removed by co-evaporation with benzene (2×6 mL) and the crude material was dissolved in DCM (5 mL). A solution of 2-amino-2-methylpropan-1-ol (0.498 g, 5.59 mmol) in DCM (1 mL) was added dropwise at 0° C. After 1.5 hours, the reaction was filtered and the solids were rinsed with DCM. The filtrate was evaporated to dryness and the crude material was purified by flash column chromatography on silica gel (0-40% EtOAc in DCM as eluent) to obtain KLS-2-013 (0.824 g, 62% yield) as a white foam.

¹H NMR (400 MHz, DMSO-D₆) δ 8.19 (s, 1H), 8.16 (s, 1H), 4.87 (t, J=5.9 Hz, 1H), 4.77 (td, J=10.8, 4.9 Hz, 1H), 4.66 (dq, J=10.6, 5.3, 4.7 Hz, 1H), 3.35 (d, J=5.3 Hz, 2H), 2.07 (ddd, J=14.5, 9.8, 5.2 Hz, 1H), 1.98-1.87 (m, 2H), 1.84-1.49 (m, 12H), 1.46-1.16 (m, 8H), 1.15 (s, 6H), 1.13-0.96 (m, 4H), 0.94 (s, 3H), 0.88 (d, J=6.5 Hz, 3H), 0.63 (s, 3H).

¹³C NMR (100 MHz, DMSO-D₆) δ 162.7, 152.1, 151.7, 63.1, 63.0, 57.6, 54.9, 44.5, 44.3, 44.1, 33.1, 31.2, 24.7, 23.8, 23.5, 23.0, 22.8, 22.5, 21.6, 17.9, 16.1, 15.6, 13.7, 13.7, 12.8, 10.8, 8.5, 5.2, 1.8, -9.9.

### Example 8: Compound of Formula I Having the Structure

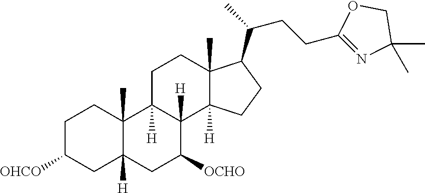

To a solution of KLS-2-013 (0.824 g, 1.59 mmol) in THF (4 mL) was added dropwise SOCl₂ (0.64 mL, 8.7 mmol) at 0° C. After stirring for 18 hours overnight, the reaction was quenched with NaHCO₃ (10 mL). The aqueous layer was extracted with Et₂O (5×10 mL) and the combined organic layers were dried over MgSO₄, filtered, and concentrated. The crude material was purified by flash column chromatography on silica gel (0-30% EtOAc in DCM as eluent) to obtain KLS-2-017 (0.505 g, 64% yield) as an off-white foam.

¹H NMR (400 MHz, CDCl₃) δ 8.02 (s, 1H), 7.98 (s, 1H), 4.90 (td, J=10.8, 5.2 Hz, 1H), 4.81 (dq, J=10.2, 5.4 Hz, 1H), 2.37-1.02 (m, 34H), 0.99 (s, 3H), 0.93 (d, J=6.4 Hz, 3H), 0.69 (s, 3H).

¹³C NMR (100 MHz, CDCl₃) δ 174.8, 161.2, 160.8, 79.1, 73.8, 73.6, 71.1, 56.4, 55.5, 55.2, 43.9, 43.8, 42.2, 40.1, 40.0, 39.6, 35.5, 34.6, 34.4, 34.2, 33.1, 33.0, 32.0, 28.6, 26.6, 26.0, 23.4, 21.4, 18.7, 12.3.

### Example 9: Compound of Formula I Having the Structure

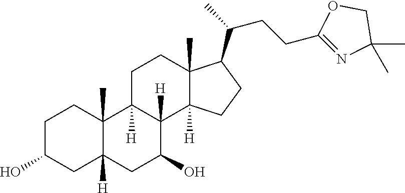

NaOH (0.078 g, 1.94 mmol) was added to a solution of KLS-2-017 (0.390 g, 0.777 mmol) in MeOH (4 mL) and the reaction was heated to reflux for 2 hours. The reaction was cooled to room temperature and diluted with EtOAc (25 mL). The organic layer was washed with water (3×10 mL), dried over MgSO₄, filtered, and concentrated. The crude material was purified by flash column chromatography on silica gel (0-100% EtOAc in DCM) to obtain KLS-2-020 (0.254 g, 73% yield) as a white foam.

¹H NMR (400 MHz, DMSO-D₆) δ 4.43 (dd, J=4.7, 2.7 Hz, 1H), 3.86 (d, J=6.8 Hz, 1H), 3.83 (d, J=1.0 Hz, 2H), 3.32-3.21 (m, 2H), 2.18 (ddd, J=14.8, 9.6, 5.1 Hz, 1H), 2.11-1.99 (m, 1H), 1.98-1.60 (m, 6H), 1.57-0.98 (m, 24H), 0.89 (d, J=6.5 Hz, 3H), 0.87 (s, 3H), 0.61 (s, 3H).

¹³C NMR (100 MHz, DMSO-D₆) δ 164.7, 77.9, 69.7, 69.4, 66.7, 64.9, 55.8, 54.5, 43.1, 43.0, 42.2, 38.7, 37.7, 37.3, 34.8, 34.7, 33.7, 31.8, 30.2, 28.3, 28.2, 28.2, 26.7, 24.3, 23.3, 20.8, 18.4, 12.0.

HRMS (ESI): m/z calcd. C₂₈H₄₈NO₃ (M+H⁺) 446.3634, found 446.3608.

FIG. 24 is a graphical representation illustrating changes in optical density over time indicative of spore germination in the presence of the compound described in Example 9 at various concentrations. The graph illustrates essentially complete inhibition of spore germination by KLS-2-020 at 0.5 mM.

### Comparative Example 17: Compound Having the Structure

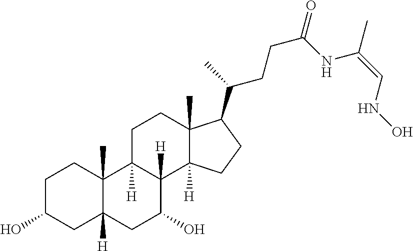

To a solution of CDCA (1.00 g, 2.55 mmol) in DCM (8.5 mL) was added HATU (1.07 g, 2.80 mmol), triethylamine (1.07 mL, 7.64 mmol), and (Z)-1-(hydroxyamino)prop-1-en-2-amine (0.449 g, 5.09 mmol). The reaction stirred at room temperature for 18 hours and was quenched with NaHCO₃ (10 mL). The organic layer was washed with NaHCO₃ (3×8 mL), dried over Na₂SO₄, filtered, and concentrated. The crude material was purified by flash column chromatography on silica gel (1-10% MeOH in EtOAc as eluent) to obtain KLS-2-086 (0.800 g, 68% yield) as a white foam.

¹H NMR (400 MHz, DMSO-D₆) δ 6.29 (s, 2H), 4.30 (d, J=4.6 Hz, 1H), 4.11 (d, J=3.3 Hz, 1H), 3.65-3.61 (m, 1H), 3.27-3.07 (m, 1H), 2.37 (ddd, J=15.4, 9.8, 5.3 Hz, 1H), 2.30-2.07 (m, 2H), 1.91 (d, J=11.7 Hz, 1H), 1.79 (ddd, J=15.2, 10.1, 3.1 Hz, 3H), 1.72 (s, 3H), 1.66 (d, J=7.7 Hz, 1H), 1.53-0.94 (m, 16H), 0.90 (d, J=6.3 Hz, 3H), 0.83 (s, 3H), 0.60 (s, 3H).

¹³C NMR (100 MHz, DMSO-D₆) δ 171.1, 155.7, 70.3, 66.1, 64.9, 55.4, 50.0, 41.9, 41.4, 35.3, 34.9, 34.8, 34.7, 34.7, 32.3, 30.7, 30.6, 29.3, 27.8, 27.8, 23.2, 22.7, 20.2, 18.2, 16.3, 15.2, 11.7.

### Example 10: Compound of Formula I Having the Structure

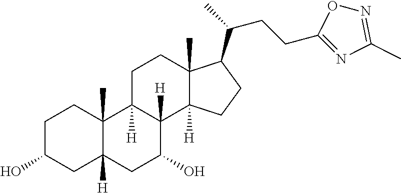

KLS-2-086 (0.300 g, 0.669 mmol) was dissolved in toluene (2.2 mL) and THF (2.2 mL). The solution was heated via microwave irradiation at 160° C. for 45 minutes. The solvent was removed under reduced pressure and the crude residue was purified by flash column chromatography on silica gel (0-10% MeOH in EtOAc as eluent) to obtain KLS-2-090 (0.164 g, 57% yield) as a white foam.

¹H NMR (400 MHz, DMSO-D₆) δ 4.30 (d, J=4.7 Hz, 1H), 4.11 (d, J=3.4 Hz, 1H), 3.62 (t, J=3.2 Hz, 1H), 3.25-3.10 (m, 1H), 2.90 (ddd, J=14.8, 10.1, 4.5 Hz, 1H), 2.84-2.73 (m, 1H), 2.29 (s, 3H), 2.19 (q, J=13.0 Hz, 1H), 1.97-1.61 (m, 7H), 1.40 (tdd, J=21.4, 13.9, 10.1 Hz, 8H), 1.29-1.06 (m, 7H), 1.00 (td, J=11.8, 6.1 Hz, 1H), 0.94 (d, J=5.6 Hz, 3H), 0.84 (s, 3H), 0.60 (s, 3H).

HRMS (ESI): m/z calcd. C₂₆H₄₂N₂NaO₃ (M+Na⁺) 453.3093, found 453.3075.

### Comparative Example 18: Compound Having the Structure

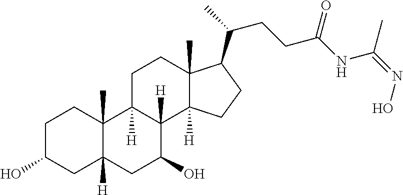

To a suspension of UDCA (1.00 g, 2.55 mmol) in DCM (8.5 mL) was added (Z)—N′-hydroxyacetimidamide (0.189 g, 2.55 mmol), HATU (1.065 g, 2.80 mmol), and diisopropylethylamine (1.3 mL, 7.6 mmol). The reaction stirred at room temperature for 24 hours and was quenched with water (5 mL). The organic phase was washed with water (2×10 mL), dried over MgSO₄, filtered, and concentrated. The crude material was purified by flash column chromatography on silica gel (1-10% MeOH in EtOAc as eluent) to obtain KLS-2-078 (0.639 g, 56% yield) as a white amorphous solid.

¹H NMR (400 MHz, DMSO-D₆) δ 6.29 (s, 2H), 4.43 (d, J=4.6 Hz, 1H), 3.87 (d, J=6.8 Hz, 1H), 3.31-3.22 (m, 2H), 2.37 (ddd, J=15.2, 9.8, 5.2 Hz, 1H), 2.24 (ddd, J=15.7, 9.3, 6.7 Hz, 2H), 1.94 (d, J=11.2 Hz, 1H), 1.90-1.73 (m, 2H), 1.72 (s, 3H), 1.70-1.58 (m, 4H), 1.56-0.93 (m, 16H), 0.90 (d, J=6.6 Hz, 3H), 0.87 (s, 3H), 0.62 (s, 3H).

¹³C NMR (100 MHz, DMSO-D₆) δ 171.1, 155.7, 69.7, 69.5, 64.9, 55.9, 54.6, 43.1, 43.0, 42.2, 38.7, 37.7, 37.3, 34.9, 34.8, 33.8, 30.7, 30.2, 29.3, 28.1, 26.7, 23.3, 20.8, 18.4, 16.3, 12.0.

### Example 11: Compound of Formula I Having the Structure

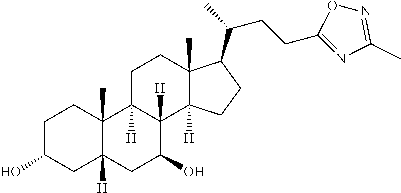

KLS-2-078 (0.300 g, 0.669 mmol) was dissolved in toluene (2.2 mL) and THF (2.5 mL). The solution was heated in the MW at 160° C. for 45 minutes. The solvent was removed under reduced pressure and the crude residue was purified by flash column chromatography on silica gel (0-10% MeOH in EtOAc as eluent) to obtain KLS-2-083 (0.155 g, 54% yield) as a white foam.

¹H NMR (400 MHz, DMSO-D₆) δ 4.43 (d, J=4.6 Hz, 1H), 3.87 (d, J=6.8 Hz, 1H), 3.31-3.23 (m, 2H), 2.90 (ddd, J=15.1, 10.0, 4.4 Hz, 1H), 2.86-2.72 (m, 2H), 2.29 (s, 3H), 2.01-1.59 (m, 7H), 1.57-1.00 (m, 16H), 0.94 (d, J=5.9 Hz, 3H), 0.87 (s, 3H), 0.61 (s, 3H).

¹³C NMR (100 MHz, DMSO-D₆) δ 180.0, 166.8, 69.8, 69.5, 55.9, 54.5, 43.2, 43.1, 42.2, 38.8, 38.8, 37.8, 37.3, 34.9, 33.8, 33.8, 32.4, 30.3, 28.2, 26.8, 23.4, 22.7, 20.9, 18.3, 12.1, 11.2.

### Comparative Example 19: Compound Having the Structure

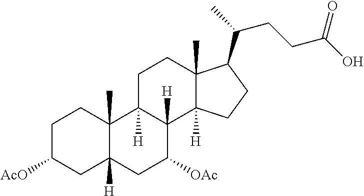

CDCA (1.00 g, 2.55 mmol) was added to a mixture of Ac₂O (2 ml, 21 mmol) and pyridine (3 ml, 37 mmol). The reaction stirred at room temperature for 3 hours and was diluted with Et₂O (20 mL). The organic layer was washed with 4 M HCl (3×10 mL). The organic layer was dried over Na₂SO₄, filtered, and concentrated to obtain KLS-2-087 (1.18 g, 97% yield) as a white foam.

¹H NMR (400 MHz, DMSO-D₆) δ 11.94 (s, 1H), 4.76 (t, J=3.0 Hz, 1H), 4.47 (tt, J=11.0, 4.3 Hz, 1H), 2.23 (ddd, J=15.0, 9.6, 5.3 Hz, 1H), 2.10 (ddd, J=15.8, 9.1, 6.9 Hz, 1H), 1.98 (s, 3H), 1.97 (s, 3H), 1.95-1.90 (m, 3H), 1.83-0.95 (m, 21H), 0.90 (s, 3H), 0.88 (d, J=6.7 Hz, 3H), 0.62 (s, 3H).

¹³C NMR (100 MHz, DMSO-D₆) δ 174.8, 169.8, 169.7, 73.4, 70.5, 64.9, 55.3, 50.0, 42.2, 37.1, 34.8, 34.7, 34.4, 34.3, 34.3, 33.6, 30.8, 30.7, 30.6, 27.5, 26.4, 23.0, 22.3, 21.3, 21.1, 20.2, 18.1, 11.5.

### Comparative Example 20: Compound Having the Structure

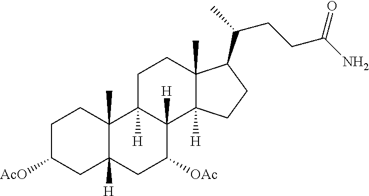

To a solution of KLS-2-087 (1.17 g, 2.455 mmol) in benzene (12 ml) was added SOCl₂ (0.90 ml, 12.3 mmol). The reaction was heated to 80° C. for 2.5 hours and the solvent was removed under reduced pressure. The residue was dissolved in benzene and concentrated under reduced pressure (3×). A 0.5 M solution of ammonia in THF (34.4 ml, 17.2 mmol) was added dropwise to the residue and stirred overnight 12 hours. The solvent was evaporated and the residue was partitioned between DCM (10 mL) and water (10 mL). The organic layer was washed with water (2×10 mL), dried over Na₂SO₄, filtered and concentrated. The crude material was purified by flash column chromatography on silica gel (10-100% EtOAc in DCM as eluent) to obtain KLS-2-088 (0.313 g, 27% yield) as an off-white foam. The mixed fractions were repurified by flash column chromatography on silica gel (20-60% EtOAc/DCM) to obtain an additional 200 mg (17% yield) of the product.

¹H NMR (400 MHz, DMSO-D₆) δ 7.21 (s, 1H), 6.63 (s, 1H), 4.76 (q, J=3.0 Hz, 1H), 4.47 (dq, J=11.4, 6.5, 5.4 Hz, 1H), 2.05 (ddd, J=14.8, 9.8, 5.3 Hz, 1H), 1.98 (s, 3H), 1.97 (s, 3H), 1.95-1.90 (m, 4H), 1.86-0.95 (m, 21H), 0.90 (s, 3H), 0.88 (d, J=6.5 Hz, 3H), 0.62 (s, 3H).

¹³C NMR (100 MHz, DMSO) δ 174.7, 169.8, 137.2, 114.8, 72.8, 59.8, 55.4, 54.4, 43.3, 39.8, 36.0, 35.3, 33.8, 33.1, 32.1, 31.3, 29.0, 28.0, 27.5, 27.0, 24.1, 22.5, 21.2, 21.1, 20.8, 18.4, 14.1, 11.8.

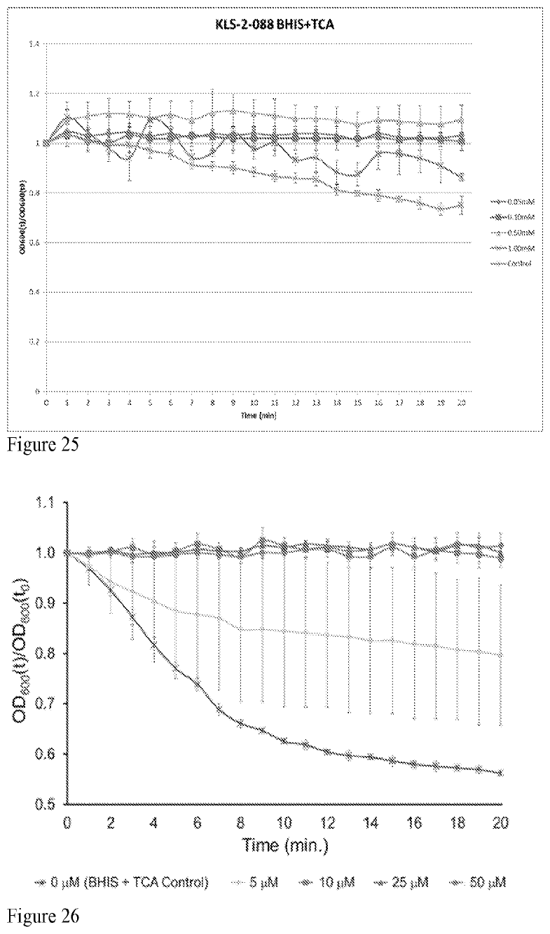

FIG. 25 is a graphical representation illustrating changes in optical density over time indicative of spore germination in the presence of an exemplary comparative compound as described in Comparative Example 20 at various concentrations. The graph illustrates essentially complete inhibition of spore germination by KLS-2-088 at 0.05 mM.

### Comparative Example 21: Compound Having the Structure

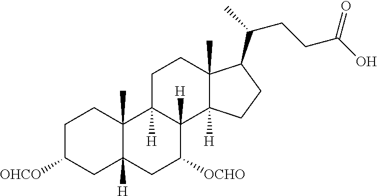

CDCA (2.00 g, 5.09 mmol) was dissolved in a mixture of formic acid (39.1 ml) and perchloric acid (0.092 ml, 1.5 mmol). The solution was heated to 50° C. for 20 minutes, then was cooled to 40° C. Acetic anhydride (20 mL) was added over 20 minutes, and the reaction was cooled to room temperature. The reaction was quenched by pouring into 100 mL of water and the aqueous phase was extracted with Et₂O (3×100 mL). The combined organic layers were washed with water (5×100 mL), brine (100 mL), dried over Na₂SO₄, filtered, and concentrated. The crude material was purified by flash column chromatography on silica gel (5-30% acetone in hexanes as eluent) to obtain KLS-3-021 (0.872 g, 38% yield) as a white foam.

¹H NMR (400 MHz, DMSO-D₆) δ 11.92 (s, 1H), 8.24 (s, 1H), 8.17 (s, 1H), 4.88 (d, J=3.4 Hz, 1H), 4.59 (tt, J=11.2, 4.5 Hz, 1H), 2.30-0.99 (m, 26H), 0.92 (s, 3H), 0.88 (d, J=6.5 Hz, 3H), 0.63 (s, 3H).

### Comparative Example 22: Compound Having the Structure

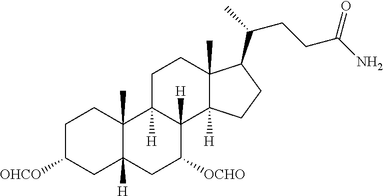

To a solution of KLS-3-021 (0.879 g, 1.959 mmol) in benzene (9.8 ml) was added SOCl₂ (0.572 ml, 7.8 mmol) and the reaction was heated to 80° C. for 4 hours.

The reaction was cooled to room temperature and the solvent was removed by rotary evaporation. The excess SOCl₂ was removed by co-evaporation with benzene (3×3 mL), and the residue was dissolved in benzene (9.8 ml). Ammonia gas was bubbled through the solution for 10 minutes at room temperature and the solvent was evaporated. The crude material was purified by flash column chromatography on silica gel (5-50% EtOAc in DCM as eluent) to obtain KLS-3-023 (0.525 g, 60% yield) as a pale yellow solid.

¹H NMR (400 MHz, DMSO-D₆) δ 8.24 (s, 1H), 8.17 (s, 1H), 7.20 (s, 1H), 6.63 (s, 1H), 4.88 (d, J=3.2 Hz, 1H), 4.59 (tt, J=10.9, 4.5 Hz, 1H), 2.12-1.00 (m, 26H), 0.92 (s, 3H), 0.88 (d, J=6.4 Hz, 3H), 0.62 (s, 3H).

¹³C NMR (100 MHz, DMSO-D₆) δ 174.7, 161.9, 161.7, 73.3, 70.8, 55.3, 49.9, 42.2, 39.0, 37.0, 34.8, 34.3, 34.2, 33.5, 31.9, 31.3, 31.1, 27.5, 26.5, 22.9, 22.3, 20.8, 20.2, 18.3, 14.1, 11.6.

### Comparative Example 23: Compound Having the Structure

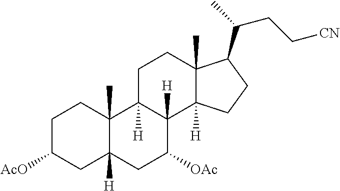

To a solution of KLS-3-023 (0.525 g, 1.17 mmol) in THF (11.7 ml) at 0° C. was added pyridine (0.19 ml, 2.3 mmol) and trifluoroacetic anhydride (TFAA, 0.34 ml, 2.4 mmol). The reaction slowly warmed to room temperature over 12 hours and was concentrated under reduced pressure. The residue was partitioned between 1M HCl (20 mL) and EtOAc (30 mL). The aqueous phase was extracted with EtOAc (2×30 mL) and the combined organic layers were dried over Na₂SO₄, filtered, and concentrated. The crude material was purified by flash column chromatography on silica gel (10-50% EtOAc in hexanes as eluent) to obtain KLS-3-026 (0.425 g, 84% yield) as a white foam.

¹H NMR (400 MHz, DMSO-D₆) δ 8.24 (s, 1H), 8.17 (s, 1H), 4.89 (q, J=2.9 Hz, 1H), 4.59 (tt, J=10.9, 4.5 Hz, 1H), 2.58-2.34 (m, 2H), 2.13-1.00 (m, 23H), 0.92 (s, 3H), 0.91 (d, J=6.7 Hz, 4H), 0.64 (s, 3H).

¹³C NMR (100 MHz, DMSO-D₆) δ 161.8, 161.7, 121.0, 73.3, 70.8, 54.9, 54.8, 49.8, 42.3, 42.3, 37.0, 34.7, 34.3, 34.3, 34.2, 33.5, 31.1, 30.9, 27.5, 26.5, 22.9, 22.3, 20.1, 17.6, 13.3, 11.5.

### Comparative Example 24: Compound Having the Structure

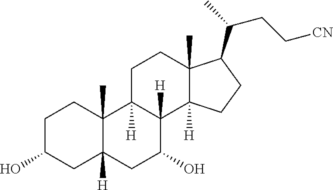

To a solution of KLS-3-026 (0.415 g, 0.966 mmol) in MeOH (4.8 ml) was added NaOH (0.097 g, 2.42 mmol) and the reaction mixture was heated to reflux for 24 hours. The solution was cooled to room temperature and the solvent was removed by rotary evaporation. The residue was partitioned between water (10 mL) and EtOAc (10 mL). The organic layer was washed with water (2×10 mL), dried over Na₂SO₄, filtered and concentrated. The crude material was purified by flash column chromatography on silica gel (0-100% EtOAc in hexanes as eluent) to obtain KLS-3-027 (0.310 g, 86% yield) as a white solid.

¹H NMR (400 MHz, DMSO-D₆) δ 4.30 (d, J=4.7 Hz, 1H), 4.12 (d, J=3.4 Hz, 1H), 3.67-3.56 (m, 1H), 3.24-3.11 (m, 1H), 2.43 (td, J=16.9, 8.4 Hz, 1H), 2.19 (q, J=13.0 Hz, 1H), 1.91 (d, J=11.5 Hz, 1H), 1.87-1.58 (m, 6H), 1.58-0.96 (m, 17H), 0.91 (d, J=6.5 Hz, 3H), 0.84 (s, 3H), 0.62 (s, 3H).

¹³C NMR (100 MHz, DMSO-D₆) δ 121.1, 70.3, 66.1, 64.9, 61.6, 55.1, 50.0, 42.0, 41.4, 35.3, 34.9, 34.8, 34.7, 32.3, 31.0, 30.5, 27.7, 23.1, 22.7, 20.2, 17.7, 15.2, 13.4, 11.6.

### Comparative Example 25: Compound Having the Structure

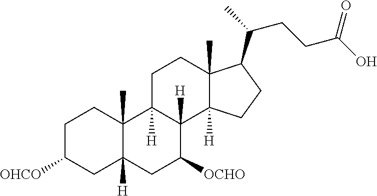

UDCA (1.00 g, 2.55 mmol) was dissolved in formic acid (20 ml, 509 mmol) and perchloric acid (0.05 ml, 0.7 mmol) was added at room temperature. The mixture was heated to 50° C. for 12 hours, then cooled to 40° C. Acetic anhydride (10 mL) was added over 10 minutes and the solution was cooled to room temperature. The reaction was quenched with water (100 mL) and diluted with Et₂₀ (200 mL). The organic layer was washed with water (5×10 mL), dried over Na₂SO₄, filtered, and concentrated. The crude material was purified by flash column chromatography on silica gel (5-30% acetone in hexanes as eluent) to obtain KLS-2-095 (0.966 g, 85% yield) as a white foam.

¹H NMR (400 MHz, DMSO-D₆) δ 11.93 (s, 1H), 8.19 (s, 1H), 8.16 (s, 1H), 4.77 (td, J=10.7, 4.9 Hz, 1H), 4.67 (p, J=5.9 Hz, 1H), 2.22 (ddd, J=15.2, 9.6, 5.3 Hz, 1H), 2.15-2.03 (m, 1H), 1.99-0.96 (m, 24H), 0.94 (s, 3H), 0.88 (d, J=6.5 Hz, 3H), 0.64 (s, 3H).

¹³C NMR (100 MHz, DMSO-D₆) δ 174.8, 162.1, 161.7, 73.1, 73.0, 54.4, 54.3, 43.1, 41.2, 39.4, 39.2, 38.5, 34.6, 33.8, 33.5, 32.8, 32.5, 30.7, 30.7, 27.9, 26.1, 25.6, 22.8, 20.8, 18.2, 11.8.

### Comparative Example 26: Compound Having the Structure

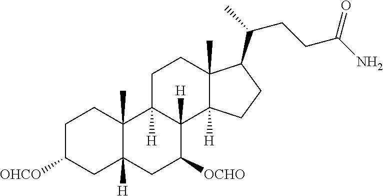

To a solution of KLS-2-095 (0.966 g, 2.15 mmol) in benzene (7.2 ml) was added SOCl₂ (0.79 ml, 10.7 mmol) and the reaction was heated to 80° C. for 4 hours. The reaction was cooled to room temperature and the solvent was removed under reduced pressure. Residual SOCl₂ was removed by co-evaporation with benzene (3×5 mL). The crude residue was dissolved in a solution of ammonia (22 ml, 10.7 mmol) in THF and was stirred at room temperature overnight. The solvent was removed under reduced pressure and the crude material was partitioned between water (20 mL) and DCM (20 mL). The organic phase was washed with water (3×10 mL), dried over Na₂SO₄, filtered, and concentrated. The crude material was purified by flash column chromatography on silica gel (10-100% acetone in hexanes as eluent) to obtain KLS-2-096 (0.430 g, 45% yield) as a white foam.

¹H NMR (400 MHz, DMSO-D₆) δ 8.19 (s, 1H), 8.16 (s, 1H), 7.37-7.10 (m, 1H), 6.63 (s, 1H), 4.77 (td, J=10.7, 4.9 Hz, 1H), 4.68 (dq, J=11.0, 5.9, 5.3 Hz, 1H), 2.04 (dt, J=10.0, 5.0 Hz, 1H), 1.99-0.96 (m, 25H), 0.94 (s, 3H), 0.88 (d, J=6.4 Hz, 3H), 0.63 (s, 3H).

¹³C NMR (100 MHz, DMSO-D₆) δ 174.6, 162.1, 161.7, 73.1, 73.0, 54.5, 54.3, 43.1, 41.2, 39.2, 38.5, 34.8, 33.8, 33.5, 32.8, 32.5, 32.0, 31.4, 30.7, 27.9, 26.1, 25.6, 22.8, 20.8, 18.4, 11.8.

### Comparative Example 27: Compound Having the Structure

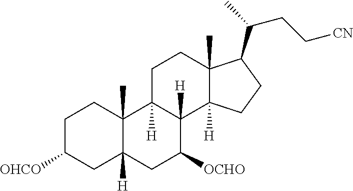

To a solution of KLS-2-096 (0.420 g, 0.938 mmol) in THF (9.4 ml) at 0° C. was added pyridine (0.15 ml, 1.8 mmol) and TFAA (0.27 ml, 1.9 mmol). The reaction was stirred for 18 hours and was concentrated under reduced pressure. The residue was partitioned between EtOAc (15 mL) and 1M HCl (10 mL). The organic layer was washed with 1M HCl (2×10 mL), dried over Na₂SO₄, filtered, and concentrated. The crude material was purified by flash column chromatography on silica gel (10-50% EtOAc in hexanes as eluent) to obtain KLS-2-098 (0.352 g, 87% yield) as a clear, colorless oil that solidified over time to a white solid.

¹H NMR (400 MHz, DMSO-D₆) δ 8.19 (s, 1H), 8.16 (s, 1H), 4.77 (td, J=10.8, 4.9 Hz, 1H), 4.67 (tt, J=10.7, 4.8 Hz, 1H), 2.60-2.33 (m, 2H), 1.98-0.98 (m, 24H), 0.94 (s, 3H), 0.91 (d, J=6.5 Hz, 3H), 0.65 (s, 3H).

¹³C NMR (100 MHz, DMSO-D₆) δ 162.1, 161.7, 121.0, 73.1, 73.0, 54.2, 54.1, 43.2, 41.2, 39.4, 38.5, 34.6, 33.8, 33.5, 33.5, 32.8, 32.5, 30.9, 27.8, 26.1, 25.6, 22.8, 20.8, 17.7, 13.4, 11.7.

### Comparative Example 28: Compound Having the Structure

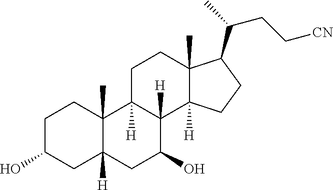

To a solution of KLS-2-098 (0.345 g, 0.80 mmol) in MeOH (4 ml) was added NaOH (0.080 g, 2.0 mmol) and the mixture was heated to reflux for 2.5 hours. The reaction was cooled to room temperature and the solvent was removed under reduced pressure. The residue was partitioned between EtOAc (10 mL) and water (10 mL). The organic layer was washed with water (2×10 mL), dried over MgSO₄, filtered, and concentrated. The crude material was purified by flash column chromatography on silica gel (50% EtOAc in hexanes as eluent) to obtain KLS-2-099 (0.211 g, 70% yield) as a white foam.

¹H NMR (400 MHz, DMSO-D₆) δ 4.43 (d, J=4.5 Hz, 1H), 3.88 (d, J=7.0 Hz, 1H), 3.33-3.19 (m, 2H), 2.62-2.30 (m, 2H), 2.00-0.96 (m, 24H), 0.91 (d, J=6.5 Hz, 3H), 0.87 (s, 3H), 0.63 (s, 3H).

¹³C NMR (100 MHz, DMSO-D₆) δ 121.1, 69.7, 69.4, 55.8, 54.3, 43.2, 43.0, 42.2, 38.7, 37.7, 37.3, 34.8, 34.8, 33.8, 33.8, 31.0, 30.2, 28.1, 26.7, 23.3, 20.8, 17.8, 13.4, 12.0.

### Example 12: Compound of Formula I Having the Structure

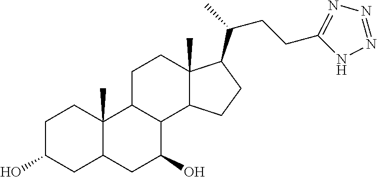 [SMILES: C[C@H](CCc1nnn[nH]1)[C@H]1CCC2C3C(CC[C@@]21C)[C@@]1(C)CC[C@@H](O)CC1C[C@@H]3O]

To a solution of KLS-2-099 (0.100 g, 0.268 mmol) in DMF (0.54 ml) was added sodium azide (NaN₃, 0.052 g, 0.80 mmol) followed by Et₃N.HCl (0.111 g, 0.80 mmol). The reaction was heated by MW at 130° C. for approximately 48 hours. The reaction was poured into a separatory funnel containing 1 M HCl (10 mL). The aqueous phase was extracted with EtOAc (3×10 mL) and the combined organic layers were dried over Na₂SO₄, filtered, and concentrated. The crude material was purified by flash column chromatography on silica gel (1-10% MeOH in DCM as eluent) to obtain the starting material (35 mg, 35% recovery) followed by the elution of KLS-3-006 (0.025 g, 22% yield).

¹H NMR (400 MHz, DMSO-D₆) δ 3.29 (ddt, J=16.1, 10.8, 5.2 Hz, 2H), 2.91 (ddd, J=14.9, 10.6, 4.6 Hz, 1H), 2.86-2.67 (m, 1H), 2.57-2.37 (m, 1H), 2.05-1.02 (m, 26H), 0.96 (d, J=5.7 Hz, 3H), 0.87 (s, 3H), 0.60 (s, 3H).

¹³C NMR (100 MHz, DMSO-D₆) δ 156.3, 69.7, 69.4, 55.8, 54.9, 54.5, 43.1, 43.0, 42.2, 38.7, 37.7, 37.3, 34.8, 34.8, 33.8, 33.5, 30.2, 28.2, 26.7, 23.3, 20.8, 19.7, 18.3, 12.0.

### Comparative Example 29: Compound Having the Structure

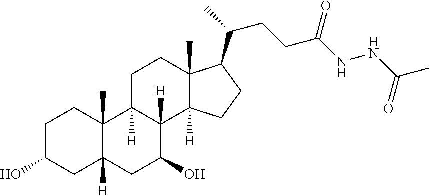

To a solution of UDCA (1.00 g, 2.55 mmol) in DCM (26 ml) was added HATU (1.26 g, 3.31 mmol) and N,N-diisopropylethylamine (DIPEA, 1.1 ml, 6.37 mmol), followed by acetohydrazide (0.252 g, 3.06 mmol). The reaction was stirred for 1 hour and 5 mL of DMF was added to help solubilize the reagents. The reaction was stirred overnight 18 hours and was quenched by the addition of saturated aqueous NaHCO₃ (20 mL). The organic layer was washed with NaHCO₃ (3×40 mL), dried over Na₂SO₄, filtered, and concentrated. The crude material was purified by flash column chromatography on silica gel (1-5% MeOH in DCM as eluent).

Pure fractions were combined and concentrated. The residue was dissolved in DCM (100 mL) and washed with 1M HCl (3×30 mL). The organic layer was dried over Na₂SO₄, filtered, and concentrated to obtain KLS-3-029 (0.778 g, 68% yield) as a white foam.

¹H NMR (400 MHz, DMSO-D₆) δ 9.66 (s, 1H), 9.62 (s, 1H), 4.43 (d, J=4.6 Hz, 1H), 3.87 (d, J=6.7 Hz, 1H), 3.32-3.21 (m, 2H), 2.12 (ddd, J=14.5, 9.6, 5.2 Hz, 1H), 2.02 (ddd, J=14.2, 9.2, 6.8 Hz, 1H), 1.98-1.89 (m, 1H), 1.83 (s, 3H), 1.80-1.55 (m, 2H), 1.55-0.97 (m, 21H), 0.89 (d, J=6.5 Hz, 3H), 0.87 (s, 3H), 0.61 (s, 3H).

¹³C NMR (100 MHz, DMSO-D₆) δ 171.4, 167.9, 69.7, 69.5, 55.9, 54.7, 48.6, 43.1, 43.0, 42.2, 38.7, 37.7, 37.3, 34.9, 34.8, 33.8, 31.4, 30.3, 30.2, 28.2, 26.7, 23.3, 20.9, 20.5, 18.4, 12.1.

### Example 13: Compound of Formula I Having the Structure

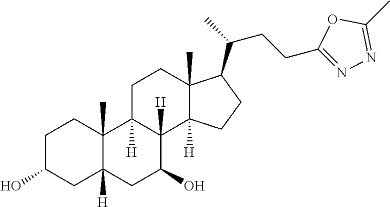

KLS-3-029 (0.300 g, 0.669 mmol) was dissolved in toluene (2.2 ml) and THF (2.2 ml). The solution was heated in the MW at 160° C. for 45 minutes. The solvent was removed under reduced pressure and the crude residue was purified by flash column chromatography on silica gel (0-10% MeOH in EtOAc as eluent) to obtain KLS-3-031 (21.5 mg, 7% yield) as a white foam.

¹H NMR (400 MHz, DMSO-D₆) δ 4.43 (d, J=4.6 Hz, 1H), 3.87 (d, J=6.8 Hz, 1H), 3.27 (d, J=22.6 Hz, 2H), 2.82 (ddd, J=14.4, 9.9, 4.3 Hz, 1H), 2.69 (ddd, J=15.4, 9.0, 6.6 Hz, 1H), 2.44 (s, 3H), 2.01-1.00 (m, 23H), 0.94 (d, J=5.9 Hz, 3H), 0.93-0.90 (m, 1H), 0.87 (s, 3H), 0.61 (s, 3H).

### Example 14: Compound of Formula I Having the Structure

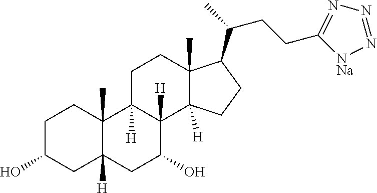

To a suspension of KLS-3-027 (0.195 g, 0.522 mmol) in toluene (1.1 ml) in a MW vial was added NaN₃ (0.102 g, 1.57 mmol) and triethylamine hydrochloride (0.258 g, 1.87 mmol). The reaction was heated via MW at 130° C. for 3 hours, then cooled to room temperature. DMF (1 mL) was added and the reaction was heated via MW at 160° C. for 4 hours, then cooled to room temperature. The reaction was quenched with water (1 mL) and 1M aqueous HCl (2 mL) was added. The precipitate was collected via vacuum filtration and rinsed with water (3 mL), then dried on the frit. To prepare the sodium salt of KLS-3-074, a 1 cm wide column was filled with 12 cm of Dowex-50 WX2 (50-100 mesh, strongly acidic) ion-exchange resin. The column was prepared by sequentially washing with 1:1 acetonitrile/water, approximately 1 M aqueous NaHCO₃ (caution: gas evolution), water, and finally 1:1 acetonitrile/water. The reaction product was dissolved in 1:1 acetonitrile/water and loaded onto the column, which was eluted with 1:1 acetonitrile/water. The fractions containing the product were lyophilized to furnish KLS-3-074 (0.078 g, 34% yield) as a white solid.

¹H NMR (400 MHz, CD₃OD) δ 3.79 (d, J=2.8 Hz, 1H), 3.38 (td, J=11.0, 5.5 Hz, 1H), 2.99 (ddd, J=15.0, 10.4, 4.3 Hz, 1H), 2.93-2.78 (m, 1H), 2.27 (q, J=12.9 Hz, 1H), 2.06-1.16 (m, 21H), 1.16-1.07 (m, 1H), 1.05 (d, J=5.9 Hz, 3H), 1.00 (dd, J=14.2, 3.3 Hz, 1H), 0.93 (s, 3H), 0.68 (s, 3H).

¹³C NMR (100 MHz, CD₃OD) δ 159.2, 72.8, 69.0, 57.1, 51.5, 43.7, 43.2, 41.0, 40.7, 40.5, 36.8, 36.5, 36.2, 35.9, 35.4, 34.0, 31.3, 29.2, 24.6, 23.4, 21.8, 21.3, 18.8, 12.1.

LC/MS (ESI): m/z calcd. C₂₄H₃₉N₄O₂ (M⁺) 415.3, found 415.4.

FIG. 26 is a graphical representation illustrating changes in optical density over time indicative of spore germination in the presence of the compound described in Example 14 at various concentrations. The graph illustrates essentially complete inhibition of spore germination by KLS-3-074 at 10 μM.

### Comparative Example 30: Compound Having the Structure

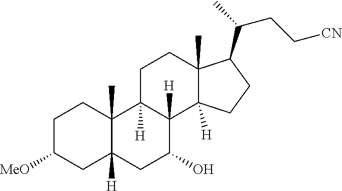

To a suspension of NaH (60% dispersion in mineral oil, 0.072 g, 1.8 mmol) in THF (3 ml) was added a solution of KLS-3-027 (0.270 g, 0.723 mmol) in THF (3 ml). After 1 hour at room temperature, MeI (0.050 ml, 0.79 mmol) was added as a solution in THF (3 ml). After 12 hours at room temperature, additional MeI (0.050 ml, 0.79 mmol) was added, and the reaction stirred an additional 24 hours. The reaction was quenched by the addition of 1M aqueous HCl (5 mL) and the aqueous layer was extracted with EtOAc (3×10 mL). The combined organic layers were washed with saturated aqueous sodium thiosulfate (10 mL), saturated aqueous NaHCO₃ (10 mL), water (10 mL) and brine (10 mL). The organic layer was dried over Na₂SO₄, filtered, and concentrated. The crude material was purified by flash column chromatography on silica gel (5-100% EtOAc in hexanes as eluent) to obtain KLS-4-003 (0.200 g, 71% yield) as a white amorphous solid.

¹H NMR (400 MHz, DMSO-D₆) δ 3.88 (d, J=6.7 Hz, 1H), 3.32-3.23 (m, 1H), 3.20 (s, 3H), 3.05 (dd, J=10.6, 5.1 Hz, 1H), 2.56-2.52 (m, 2H), 2.41 (dt, J=16.8, 8.1 Hz, 1H), 1.99-0.94 (m, 22H), 0.91-0.89 (m, 1H), 0.90 (d, J=6.9 Hz, 3H), 0.89 (s, 3H), 0.63 (s, 3H).

¹H NMR (400 MHz, CD₂C₁₂) δ 3.52 (ddd, J=11.5, 9.0, 5.1 Hz, 1H), 3.28 (s, 3H), 3.07 (dp, J=10.2, 4.7 Hz, 1H), 2.36 (ddd, J=17.0, 8.8, 5.2 Hz, 1H), 2.25 (dt, J=16.7, 8.2 Hz, 1H), 2.07-0.97 (m, 24H), 0.92 (d, J=7.4 Hz, 3H), 0.91 (s, 3H), 0.68 (s, 3H).

¹³C NMR (100 MHz, CD₂C₁₂) δ 120.7, 80.5, 71.5, 56.1, 55.7, 55.1, 44.2, 44.1, 42.9, 40.5, 39.4, 37.7, 35.6, 35.2, 34.7, 34.2, 32.0, 28.9, 27.3, 27.1, 23.6, 21.5, 18.2, 14.6, 12.3.

### Comparative Example 31: Compound Having the Structure

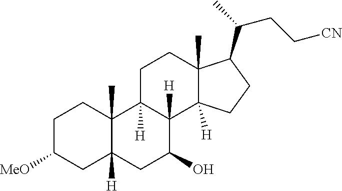

To a suspension of NaH (60% dispersion in mineral oil, 0.058 g, 1.46 mmol) in THF (3 ml) was added a solution of KLS-2-099 (0.450 g, 1.21 mmol) in THF (3 ml). After 1 hour at room temperature, MeI (0.09 ml, 1.5 mmol) was added as a solution in THF (3 ml). After 12 hours at room temperature, additional MeI (0.09 ml, 1.5 mmol) was added, and the reaction stirred an additional 24 hours. The reaction was quenched by the addition of 1M aqueous HCl (5 mL) and the aqueous layer was extracted with EtOAc (3×10 mL). The combined organic layers were washed with saturated aqueous sodium thiosulfate (10 mL), saturated aqueous NaHCO₃ (10 mL), water (10 mL) and brine (10 mL). The organic layer was dried over Na₂SO₄, filtered, and concentrated. The crude material was purified by flash column chromatography on silica gel (5-100% EtOAc in hexanes as eluent) to obtain KLS-4-018 (0.357 g, 76% yield) as a white amorphous solid.

¹H NMR (400 MHz, CDCl₃) δ 3.84 (d, J=3.4 Hz, 1H), 3.34 (s, 3H), 3.02 (tt, J=11.1, 4.3 Hz, 1H), 2.37 (ddd, J=16.9, 8.5, 5.1 Hz, 1H), 2.27 (dt, J=16.8, 8.2 Hz, 1H), 2.17-1.05 (m, 23H), 1.00-0.93 (m, 1H), 0.97 (d, J=6.6 Hz, 3H), 0.91 (s, 3H), 0.68 (s, 3H).

¹³C NMR (100 MHz, CDCl₃) δ 120.2, 80.5, 68.4, 55.5, 55.5, 50.4, 42.8, 41.4, 39.6, 39.4, 35.9, 35.4, 35.3, 35.2, 34.8, 32.8, 31.5, 28.2, 27.0, 23.7, 22.8, 20.5, 17.9, 14.3, 11.8.

TLC-MS (ESI): m/z calcd. C₂₅H₄₂NO₂ (M+H⁺) 388.3, found 388.7.

### Example 15: Compound of Formula I Having the Structure

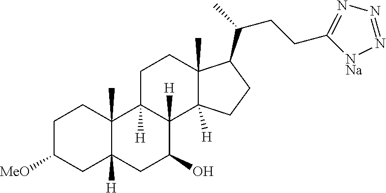

To a suspension of KLS-4-018 (0.150 g, 0.387 mmol) in isopropanol (iPrOH, 2 mL) was added zinc bromide (0.096 g, 0.43 mmol) and sodium azide (0.028 g, 0.43 mmol) in water (2 mL). The reaction was heated via MW at 160° C. for 9 hours. Starting material remained, and additional zinc bromide (0.096 g, 0.43 mmol) and sodium azide (0.028 g, 0.43 mmol) were added and the reaction was heated for 5 hours. The reaction was poured into 1 M aqueous HCl (10 mL) and the aqueous layer was extracted with EtOAc (4×10 mL). The combined organic layers were dried over Na₂SO₄, filtered, and concentrated. The crude material was purified by flash column chromatography on silica gel (2-10% MeOH in DCM as eluent) to obtain the tetrazole (0.086 g, 52% yield) as a white solid. To prepare the sodium salt of KLS-4-019, a 1 cm wide column was filled with 12 cm of Dowex-50 WX2 (50-100 mesh, strongly acidic) ion-exchange resin. The column was prepared by sequentially washing with 1:1 acetonitrile/water, approximately 1 M aqueous NaHCO₃ (caution: gas evolution), water, and finally 1:1 acetonitrile/water. The reaction product was dissolved in 1:1 acetonitrile/water and loaded onto the column, which was eluted with 1:1 acetonitrile/water. The fractions containing the product were lyophilized to furnish KLS-4-019 as a white solid.

¹H NMR (400 MHz, CD₃OD) δ 3.50-3.39 (m, 1H), 3.33 (s, 3H), 3.16 (td, J=10.7, 4.8 Hz, 1H), 3.02-2.92 (m, 1H), 2.81 (ddd, J=15.0, 9.4, 6.6 Hz, 1H), 2.74-2.62 (m, 1H), 2.04 (dt, J=12.4, 3.1 Hz, 2H), 1.98-1.09 (m, 20H), 1.04 (d, J=6.0 Hz, 3H), 1.00 (dd, J=10.8, 3.4 Hz, 1H), 0.97 (s, 3H), 0.70 (s, 3H).

¹³C NMR (100 MHz, CD₃OD) δ 160.6, 81.7, 71.9, 57.4, 56.4, 55.9, 44.8, 44.5, 43.9, 41.5, 40.7, 38.6, 36.7, 35.9, 35.8, 35.4, 34.7, 29.6, 27.9, 27.7, 23.9, 22.4, 21.8, 19.0, 12.6.

TLC-MS (ESI): m/z calcd. C₂₅H₄₁N₄O₂ (M⁺) 429.3, found 429.7.

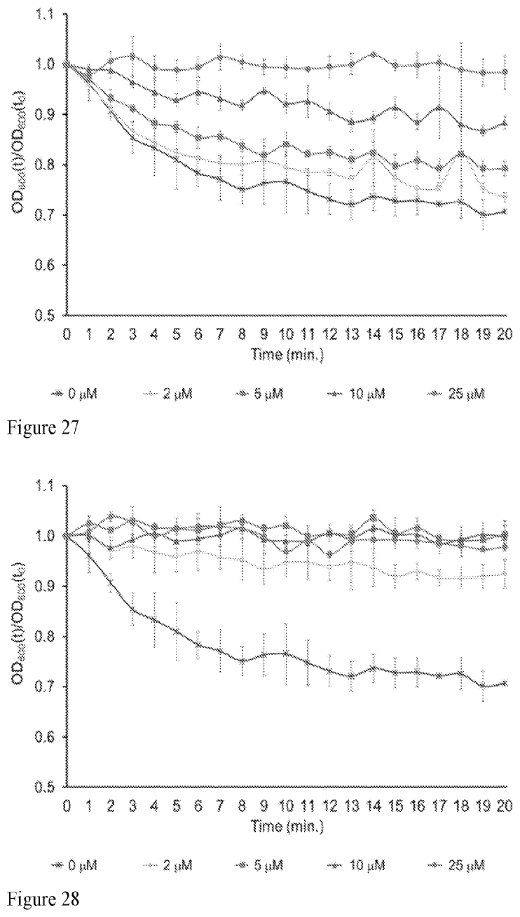

FIG. 27 is a graphical representation illustrating changes in optical density over time indicative of spore germination in the presence of the compound described in Example 15 at various concentrations. The graph illustrates essentially complete inhibition of spore germination by KLS-4-019 at 25 M.

### Example 16: Compound of Formula I Having the Structure

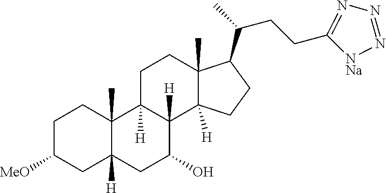

To a suspension of KLS-4-003 (0.150 g, 0.387 mmol) in iPrOH (2 mL) was added zinc bromide (0.096 g, 0.43 mmol) and sodium azide (0.028 g, 0.43 mmol) in water (2 mL). The reaction was heated via MW at 160° C. for 9 hours. Starting material remained, and additional zinc bromide (0.096 g, 0.43 mmol) and sodium azide (0.028 g, 0.43 mmol) were added and the reaction was heated for 5 hours. The reaction was poured into 1 M aqueous HCl (10 mL) and the aqueous layer was extracted with EtOAc (4×10 mL). The combined organic layers were dried over Na₂SO₄, filtered, and concentrated. The crude material was purified by flash column chromatography on silica gel (2-10% MeOH in DCM as eluent) to obtain the tetrazole (0.086 g, 52% yield) as a white solid. To prepare the sodium salt of KLS-4-004, a 1 cm wide column was filled with 12 cm of Dowex-50 WX2 (50-100 mesh, strongly acidic) ion-exchange resin. The column was prepared by sequentially washing with 1:1 acetonitrile/water, approximately 1 M aqueous NaHCO₃ (caution: gas evolution), water, and finally 1:1 acetonitrile/water. The reaction product was dissolved in 1:1 acetonitrile/water and loaded onto the column, which was eluted with 1:1 acetonitrile/water. The fractions containing the product were lyophilized to furnish KLS-4-004 as a white solid.

¹H NMR (400 MHz, CD₃OD) δ 3.80 (s, 1H), 3.33 (s, 3H), 3.06 (dq, J=10.7, 5.4, 4.1 Hz, 1H), 3.00-2.88 (m, 1H), 2.88-2.75 (m, 1H), 2.28-1.16 (m, 22H), 1.14-1.08 (m, 1H), 1.05 (d, J=5.8 Hz, 3H), 1.03-0.96 (m, 1H), 0.94 (s, 3H), 0.69 (s, 3H).

¹³C NMR (100 MHz, CD₃OD) δ 156.0, 82.4, 69.0, 57.3, 55.6, 51.5, 43.7, 43.1, 41.0, 40.8, 37.0, 36.8, 36.5, 36.3, 36.1, 35.8, 34.0, 29.3, 28.0, 24.6, 23.4, 22.3, 21.8, 18.9, 12.1.

HRMS (ESI): m/z calcd. C₂₅H₄₂N₄NaO₂ (M+Na⁺) 453.3206, found 453.3164.

FIG. 28 is a graphical representation illustrating changes in optical density over time indicative of spore germination in the presence of the compound described in Example 16 at various concentrations. The graph illustrates essentially complete inhibition of spore germination by KLS-4-004 at 5 μM.

### Example 17: Compound of Formula I Having the Structure

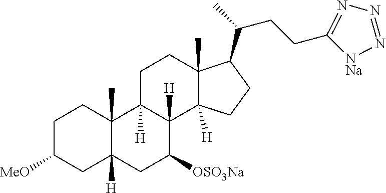

To a solution of KLS-4-019 (0.060 g, 0.14 mmol) in pyridine (0.7 mL) was added dropwise chlorosulfonic acid (0.06 mL, 0.9 mmol). The reaction was heated to 50° C. for 30 minutes and was cooled to room temperature. The reaction was quenched by the addition of 1 mL of water and the solvents were removed by rotary evaporation. The crude material was dissolved in DMSO (0.5 mL) and 1M triethylammonium acetate buffer (0.1 mL) and purified by flash column chromatography (5-100% 20 mM triethylammonium acetate buffer in acetonitrile in water as eluent, C₁₈ column) to yield a white solid after lyophilization. To prepare the sodium salt of KLS-4-048, a 1 cm wide column was filled with 12 cm of Dowex-50 WX2 (50-100 mesh, strongly acidic) ion-exchange resin. The column was prepared by sequentially washing with 1:1 acetonitrile/water, approximately 1 M aqueous NaHCO₃ (caution: gas evolution), water, and finally 1:1 acetonitrile/water. The reaction product was dissolved in 1:1 acetonitrile/water and loaded onto the column, which was eluted with 1:1 acetonitrile/water. The fractions containing the product were lyophilized to furnish KLS-4-023 as an off-white solid (0.025 g, 32% yield) as the di-sodium salt.

¹H NMR (400 MHz, CD₃OD) δ 4.28 (td, J=10.8, 5.0 Hz, 1H), 3.33 (s, 3H), 3.16 (dt, J=10.8, 5.8 Hz, 1H), 2.93 (ddd, J=14.7, 10.7, 4.4 Hz, 1H), 2.86-2.63 (m, 1H), 2.28-1.10 (m, 21H), 1.06 (s, 1H), 1.04 (d, J=5.8 Hz, 3H), 0.98 (s, 3H), 0.69 (s, 3H).

¹³C NMR (100 MHz, CD₃OD) δ 161.4, 81.6, 80.8, 56.5, 56.4, 55.8, 44.9, 43.7, 42.7, 41.2, 40.7, 36.8, 36.0, 35.8, 35.3, 35.0, 34.4, 29.7, 27.8, 27.1, 23.9, 22.4, 22.2, 19.1, 12.5.

TLC-MS (ESI): m/z calcd. C₂₅H₄₁N₄O₅S (M⁺) 509.3, found 509.5.

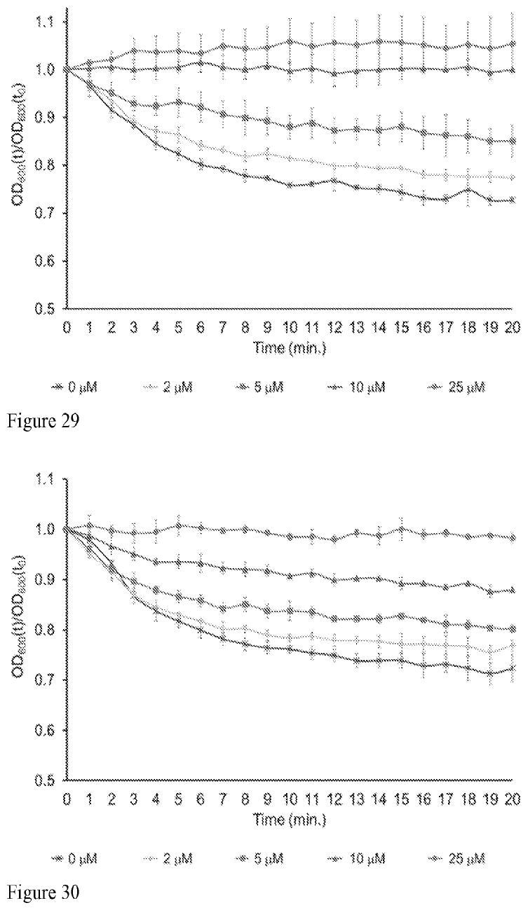

FIG. 29 is a graphical representation illustrating changes in optical density over time indicative of spore germination in the presence of the compound described in Example 17 at various concentrations. The graph illustrates essentially complete inhibition of spore germination by KLS-4-023 at 10 M.

### Example 18: Compound of Formula I Having the Structure

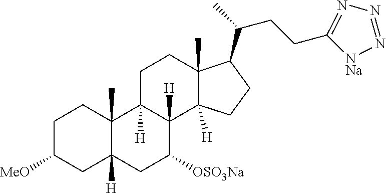

To a solution of KLS-4-004 (0.100 g, 0.23 mmol) in pyridine (1.1 mL) was added dropwise chlorosulfonic acid (0.10 mL, 1.5 mmol). The reaction was heated to 50° C. for 30 minutes and was cooled to room temperature. The reaction was quenched by the addition of 1 mL of water and the solvents were removed by rotary evaporation. The crude material was dissolved in DMSO (0.5 mL) and 1M triethylammonium acetate buffer (0.1 mL) and purified by flash column chromatography (5-100% 20 mM triethylammonium acetate buffer in acetonitrile in water as eluent, C₁₈ column) to yield a white solid after lyophilization. To prepare the sodium salt of KLS-4-008, a 1 cm wide column was filled with 12 cm of Dowex-50 WX2 (50-100 mesh, strongly acidic) ion-exchange resin. The column was prepared by sequentially washing with 1:1 acetonitrile/water, approximately 1 M aqueous NaHCO₃ (caution: gas evolution), water, and finally 1:1 acetonitrile/water. The reaction product was dissolved in 1:1 acetonitrile/water and loaded onto the column, which was eluted with 1:1 acetonitrile/water. The fractions containing the product were lyophilized to furnish KLS-4-008 as an off-white solid (0.039 g, 30% yield) as the di-sodium salt.

¹H NMR (400 MHz, DMSO-D₆) δ 4.11 (d, J=3.1 Hz, 1H), 3.19 (s, 3H), 2.98-2.82 (m, 2H), 2.76 (dt, J=15.1, 8.0 Hz, 1H), 2.21-1.02 (m, 23H), 0.95 (d, J=5.7 Hz, 3H), 0.90-0.79 (m, 1H), 0.85 (s, 3H), 0.58 (s, 3H).

¹³C NMR (100 MHz, CD₃OD) δ 158.8, 82.4, 78.2, 56.9, 55.6, 51.2, 43.8, 43.0, 40.8, 40.7, 36.7, 36.4, 36.2, 36.0, 35.2, 34.7, 31.8, 29.2, 28.3, 24.4, 23.3, 21.8, 21.1, 18.8, 12.2.

HRMS (ESI): m/z calcd. C₂₅H₄₁N₄Na₂O₅S (M+H⁺) 555.2593, found 555.2586.

FIG. 30 is a graphical representation illustrating changes in optical density over time indicative of spore germination in the presence of the compound described in Example 18 at various concentrations. The graph illustrates essentially complete inhibition of spore germination by KLS-4-008 at 25 μM.

Phase-Contrast Spore Count Assay

Experiments were run using the procedure described herein for phase-contrast spore count assay, and the results are reported in Table 1.

TABLE 1
Percent germination of NAP1 spores in the presence of 2000 μM taurocholic acid and
10 μM bile acid analogs after 20 minutes.

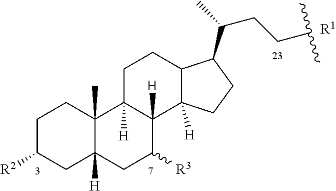

| | | | | Percent Spore Germination^{a} | | |
|---|---|---|---|---|---|---|
| Example | R¹ | R² | R³ | t₀ | t₂₀ | Relative to control^{b} |
| C.E. 1 | —CO₂Me | —OH | —αOH | 25 ± 6 | 71 ± 1 | 73 ± 1 |
| C.E. 10 | —CO₂H | —OMe | —βOMe | 25 ± 4 | 71 ± 2 | 75 ± 2 |
| Ex. 4 Ex. 7 | 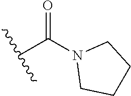 [SMILES: *C(=O)N1CCCC1] | —OMe —OMe | —βOH —βOSO₃Na | 18 ± 3 18 ± 2 | 33 ± 6 30 ± 6 | 15 ± 6 9 ± 5 |
| Ex. 14 Ex. 12 Ex. 16 Ex. 15 Ex. 18 Ex. 17 | 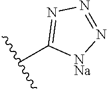 | —OH —OH —OMe —OMe —OMe —OMe | —αOH —βOH —αOH —βOH —αOSO₃Na —βOSO₃Na | 15 ± 6 17 ± 4 16 ± 4 16 ± 4 20 ± 11 21 ± 3 | 22 ± 6 29 ± 4 21 ± 4 31 ± 4 48 ± 11 44 ± 3 | 12 ± 6 19 ± 4 8 ± 4 26 ± 4 51 ± 11 42 ± 3 |

The data is Table 1 indicates that C.E.1 and C.E.10 are weak inhibitors of spore germination. The data in Table 1 also indicates that Examples 4, 7, 14, and 16 are potent inhibitors of spore germination. The data in Table 1 further indicates that Examples 12, 15, 17, and 18 are moderate inhibitors of spore germination.

The complete disclosure of all patents, patent applications, and publications, and electronically available material (e.g., GenBank amino acid and nucleotide sequence submissions; and protein data bank (pdb) submissions) cited herein are incorporated by reference. The foregoing detailed description and examples have been given for clarity of understanding only. No unnecessary limitations are to be understood therefrom. The disclosure is not limited to the exact details shown and described, for variations obvious to one skilled in the art will be included within the disclosure defined by the claims.

1. A compound of any of Formula I, II, III, IV, or V: 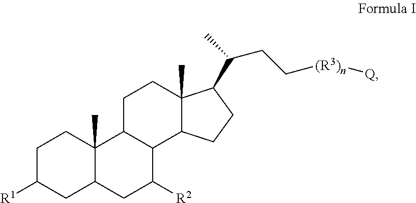
wherein:
R¹ and R² are each independently selected from the group consisting of —H, —OR⁶, and —OC(O)R⁸;
each R⁶ is independently selected from the group consisting of —H, a C1-C10 straight chain or branched chain alkyl or cycloalkyl group, —SO₃H, and a pharmaceutically acceptable salt of —SO₃H;
R⁸ represents —H or a C1-C10 straight chain or branched chain alkyl or cycloalkyl group;
R³ represents —C(O)— or a C1-C10 straight chain or branched chain alkylene or cycloalkylene group;
n=0 or 1; and
Q represents a heterocyclic ring, with the proviso that if R² is H, then Q does not represent a tetrazole; 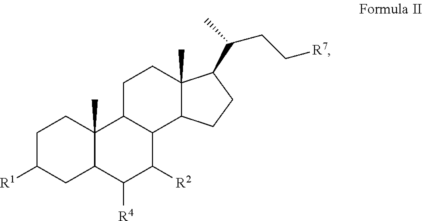
wherein:
R¹ and R² are each independently selected from the group consisting of —H, —OR⁶, and —OC(O)R⁸;
R⁴ represents —OR⁸ or —OC(O)R⁸;
each R⁶ is independently selected from the group consisting of —H, a C1-C10 straight chain or branched chain alkyl or cycloalkyl group, —SO₃H, and a pharmaceutically acceptable salt of —SO₃H;
R⁷ represents —C(O)OR⁸ or —(R³)ₙ-Q;
each R⁸ independently represents —H or a C1-C10 straight chain or branched chain alkyl or cycloalkyl group;
R³ represents —C(O)— or a C1-C10 straight chain or branched chain alkylene or cycloalkylene group;
n=0 or 1; and
Q represents a heterocyclic ring; 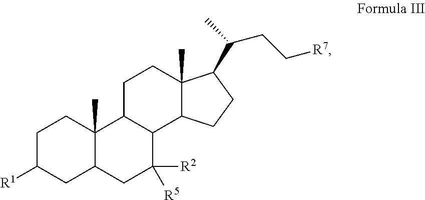
wherein:
R¹ represents H, —OR⁶, or —OC(O)R⁸;
R² represents —OR⁶ or —OC(O)R⁶;
R⁵ represents F or a C1-C10 straight chain or branched chain alkyl or cycloalkyl group;
each R⁶ is independently selected from the group consisting of —H, a C1-C10 straight chain or branched chain alkyl or cycloalkyl group, —SO₃H, and a pharmaceutically acceptable salt of —SO₃H;
R⁷ represents —C(O)OR⁸ or —(R³)ₙ-Q;
each R⁸ independently represents —H or a C1-C10 straight chain or branched chain alkyl or cycloalkyl group; 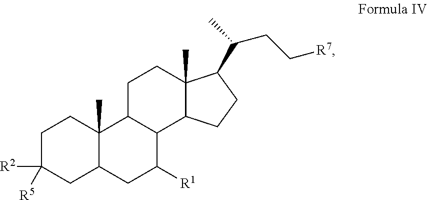
wherein:
R¹ represents H, —OR⁶, or —OC(O)R⁸;
R² represents —OR⁶ or —OC(O)R⁶;
R⁵ represents F or a C1-C10 straight chain or branched chain alkyl or cycloalkyl group;
each R⁶ is independently selected from the group consisting of —H, a C1-C10 straight chain or branched chain alkyl or cycloalkyl group, —SO₃H, and a pharmaceutically acceptable salt of —SO₃H;
R⁷ represents —C(O)OR⁸ or —(R³)ₙ-Q;
each R⁸ independently represents —H or a C1-C10 straight chain or branched chain alkyl or cycloalkyl group;
R³ represents —C(O)— or a C1-C10 straight chain or branched chain alkylene or cycloalkylene group;
n=0 or 1; and
Q represents a heterocyclic ring
R³ represents —C(O)— or a C1-C10 straight chain or branched chain alkylene or cycloalkylene group;
n=0 or 1; and
Q represents a heterocyclic ring; 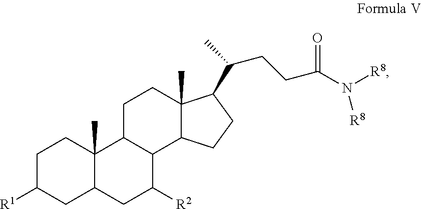
wherein:
R¹ and R² are each independently selected from the group consisting of —H, —OR⁶, and —OC(O)R⁸;
each R⁶ is independently selected from the group consisting of —H, a C1-C10 straight chain or branched chain alkyl or cycloalkyl group, —SO₃H, and a pharmaceutically acceptable salt of —SO₃H; and
each R⁸ independently represents —H or a C1-C10 straight chain or branched chain alkyl or cycloalkyl group;
with the proviso that one or both of R¹ and R² represent —OSO₃H or a pharmaceutically acceptable salt of —OSO₃H.

2. (canceled)

3. The compound of claim 1, wherein the compound is of formula II, wherein R⁴ is —OH.

4. (canceled)

5. (canceled)

6. The compound of claim 1, wherein the compound is of formula III, and R⁵ is methyl.

7. The compound of claim 1, wherein the heterocyclic ring is a three to seven-membered ring comprising 1 to 6 heteroatoms.

8. The compound of claim 1, wherein the heterocyclic ring is a five or six-membered ring comprising 1 to 4 heteroatoms.

9. The compound of claim 1, wherein each of the heteroatoms are independently selected from the group consisting of N, O, and S.

10. The compound of claim 1, wherein the heterocyclic ring is selected from the group consisting of a pyrrolidine, an oxazoline, a tetrazole, an oxadiazole, an imidazole, a trizole, an oxazole, a pyrazole, a thiazole, an isothiazole, an isoxazole, a piperidine, a piperazine, a pyridine, a morpholine, and a pyrimidine.

11. The compound of claim 1, wherein the heterocyclic ring is selected from the group consisting of: 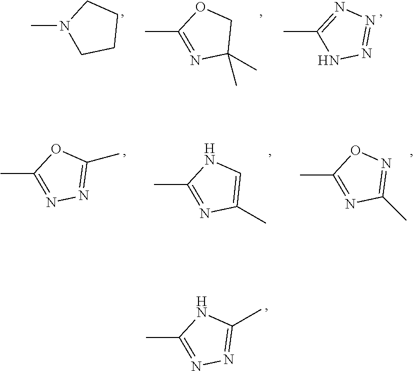
and combinations thereof.

12. (canceled)

13. A pharmaceutically acceptable salt of a compound according to claim 1.

14. A composition comprising a compound according to claim 1.

15. The composition of claim 14 further comprising a vehicle, an encapsulant, and/or an adjuvant.

16. A method of preventing a Clostridium-associated disease in a mammalian subject, comprising administering to a mammalian subject at risk of developing Clostridium-associated disease an effective amount of a compound, a pharmaceutically acceptable salt of a compound, or a composition according to any of Formulae I, II, III, IV, or V: 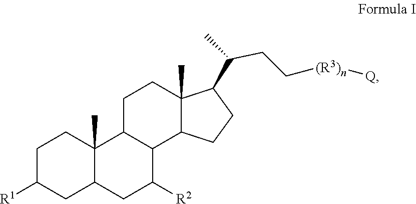
wherein:
R¹ and R² are each independently selected from the group consisting of —H, —OR⁶, and —OC(O)R⁸;
each R⁶ is independently selected from the group consisting of —H, a C1-C10 straight chain or branched chain alkyl or cycloalkyl group, —SO₃H, and a pharmaceutically acceptable salt of —SO₃H;
R⁸ represents —H or a C1-C10 straight chain or branched chain alkyl or cycloalkyl group;
R³ represents —C(O)— or a C1-C10 straight chain or branched chain alkylene or cycloalkylene group;
n=0 or 1; and
Q represents a heterocyclic ring, with the proviso that if R² is H, then Q does not represent a tetrazole; 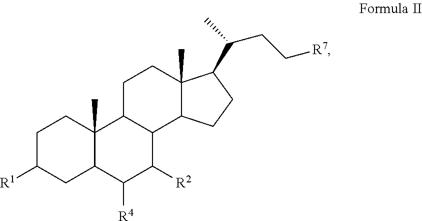
wherein:
R¹ and R² are each independently selected from the group consisting of —H, —OR⁶, and —OC(O)R⁸;
R⁴ represents —OR⁸ or —OC(O)R⁸;
each R⁶ is independently selected from the group consisting of —H, a C1-C10 straight chain or branched chain alkyl or cycloalkyl group, —SO₃H, and a pharmaceutically acceptable salt of —SO₃H;
R⁷ represents —C(O)OR⁸ or —(R³)ₙ-Q;
each R⁸ independently represents —H or a C1-C10 straight chain or branched chain alkyl or cycloalkyl group;
R³ represents —C(O)— or a C1-C10 straight chain or branched chain alkylene or cycloalkylene group;
n=0 or 1; and
Q represents a heterocyclic ring; 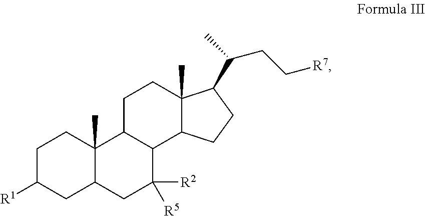
wherein:
R¹ represents H, —OR⁶, or —OC(O)R⁸;
R² represents —OR⁶ or —OC(O)R⁶;
R⁵ represents F or a C1-C10 straight chain or branched chain alkyl or cycloalkyl group;
each R⁶ is independently selected from the group consisting of —H, a C1-C10 straight chain or branched chain alkyl or cycloalkyl group, —SO₃H, and a pharmaceutically acceptable salt of —SO₃H;
R⁷ represents —C(O)OR⁸ or —(R³)ₙ-Q;
each R⁸ independently represents —H or a C1-C10 straight chain or branched chain alkyl or cycloalkyl group; 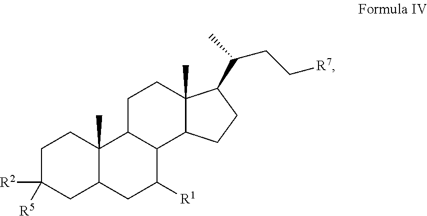
wherein:
R¹ represents H, —OR⁶, or —OC(O)R⁸;
R² represents —OR⁶ or —OC(O)R⁶;
R⁵ represents F or a C1-C10 straight chain or branched chain alkyl or cycloalkyl group;
each R⁶ is independently selected from the group consisting of —H, a C1-C10 straight chain or branched chain alkyl or cycloalkyl group, —SO₃H, and a pharmaceutically acceptable salt of —SO₃H;
R⁷ represents —C(O)OR⁸ or —(R³)ₙ-Q;
each R⁸ independently represents —H or a C1-C10 straight chain or branched chain alkyl or cycloalkyl group;
R³ represents —C(O)— or a C1-C10 straight chain or branched chain alkylene or cycloalkylene group;
n=0 or 1; and
Q represents a heterocyclic ring;
R³ represents —C(O)— or a C1-C10 straight chain or branched chain alkylene or cycloalkylene group;
n=0 or 1; and
Q represents a heterocyclic ring; 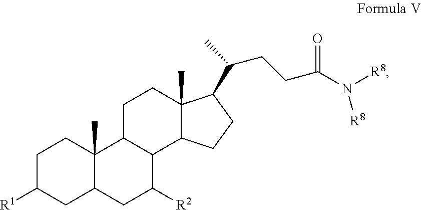
wherein:
R¹ and R² are each independently selected from the group consisting of —H, —OR⁶, and —OC(O)R⁸;
each R⁶ is independently selected from the group consisting of —H, a C1-C10 straight chain or branched chain alkyl or cycloalkyl group, —SO₃H, and a pharmaceutically acceptable salt of —SO₃H; and
each R⁸ independently represents —H or a C1-C10 straight chain or branched chain alkyl or cycloalkyl group;
with the proviso that one or both of R¹ and R² represent —OSO₃H or a pharmaceutically acceptable salt of —OSO₃H
prior to or concurrently with an optional administration of an antibiotic.

17. (canceled)

18. (canceled)

19. The method of claim 16 wherein the Clostridium-associated disease is a Clostridium difficile-associated disease or a Clostridium perfringens-associated disease.

20. The method of claim 19 wherein the C. difficile-associated disease is C. difficile colitis.

21. The method of claim 19 wherein the C. difficile-associated disease is pseudomembranous colitis.

22. The method of claim 16 wherein the subject is a human.

23. A method of preventing a Clostridium-associated disease in a mammalian subject, treating a Clostridium-associated disease in a mammalian subject, reducing the risk of developing a Clostridium-associated disease in a mammalian subject receiving antibiotic therapy, or a combination thereof, comprising administering to a mammalian subject an effective amount of a compound, a pharmaceutically acceptable salt of a compound, or a composition thereof, prior to or concurrently with an optional administration of an antibiotic, wherein the compound is of the formula: 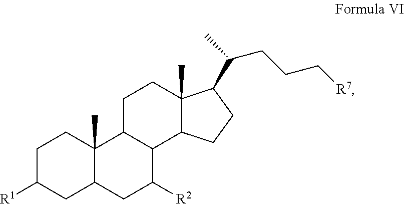
wherein:
R¹ and R² are each independently selected from the group consisting of —H, —OR⁶, and —OC(O)R⁸;
each R⁶ is independently selected from the group consisting of —H, a C1-C10 straight chain or branched chain alkyl or cycloalkyl group, —SO₃H, and a pharmaceutically acceptable salt of —SO₃H;
R⁷ represents —C(O)OR⁸ or —C(O)N(R⁸)₂; and
each R⁸ independently represents —H or a C1-C10 straight chain or branched chain alkyl or cycloalkyl group;
with the proviso that one or both of R¹ and R² represent —OSO₃H or a pharmaceutically acceptable salt of —OSO₃H.

24. (canceled)

25. (canceled)

26. The method of claim 23 wherein the Clostridium-associated disease is a Clostridium difficile-associated disease or a Clostridium perfringens-associated disease.

27. The method of claim 26 wherein the C. difficile-associated disease is C. difficile colitis.

28. The method of claim 26 wherein the C. difficile-associated disease is pseudomembranous colitis.

29. The method of claim 23 wherein the subject is a human.
